(19) United States
(12) Patent Application Publication
(10) Pub. No.: US 2022/0064170 A1
(43) Pub. Date: Mar. 3, 2022

(54) BICYCLIC HETEROCYCLIC DERIVATIVES AND THEIR USE AS PHARMACEUTICALS

(71) Applicant: Prelude Therapeutics, Incorporated, Wilmington, DE (US)

(72) Inventor: Jincong ZHUO, Garnet Valley, PA (US)

(21) Appl. No.: 17/413,676

(22) PCT Filed: Dec. 13, 2019
(86) PCT No.: PCT/US2019/066311

§ 371 Date: Jun. 14, 2021

Related U.S. Application Data
(60) Provisional application No. 62/780,001, filed on Dec. 14, 2018.

(51) Int. Cl. C07D 487/04

(52) U.S. Cl. CPC C07D 487/04

(57) ABSTRACT

The disclosure is directed to compounds of Formula I. Pharmaceutical compositions comprising compounds of Formula I, as well as methods of their use and preparation, are also described.

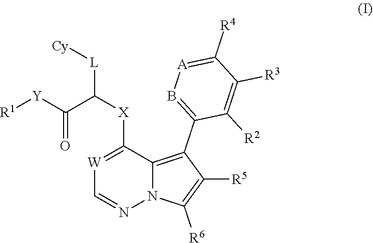

### CROSS-REFERENCE TO RELATED APPLICATION

This application claims the benefit of priority to U.S. Provisional Patent Application No. 62/780,001, filed on Dec. 14, 2018, the entirety of which is incorporated by reference herein in its entirety.

### TECHNICAL FIELD

The disclosure is directed to MCL-1 inhibitors and methods of their use.

### BACKGROUND

Apoptosis (programmed cell death) is a highly conserved cellular process that is required for embryonic development and normal tissue homeostasis (Ashkenazi A. et al., Nat. Rev. Drug Discov. 2017, 16, 273-284). Apoptotic-type cell death involves morphological changes such as condensation of the nucleus, DNA fragmentation as well as biochemical phenomena such as the activation of caspases which cause damage to key structural components of the cell, resulting in its disassembly and death. Regulation of the process of apoptosis is complex and involves the activation or repression of several intracellular signaling pathways (Cory S. et al., Nature Review Cancer 2002, 2, 647-656; Thomas L. W. et al., FEBS Lett. 2010, 584, 2981-2989; Adams J. M. et al., Oncogene 2007, 26, 1324-1337)

The Bcl-2 protein family, which includes both pro-apoptotic and anti-apoptotic members, plays a pivotal role in the regulation of the apoptosis process (Youle R. J. et al., Nat. Rev. Mol. Cell Biol. 2008, 9, 47-59; Kelly G. L. et al., Adv. Cancer Res. 2011, 111, 39-96). Bcl-2, Bcl-XL, Bcl-W, Mcl-1 and A1 are anti-apoptotic proteins and they share a common BH regions. In contrast, the pro-apoptotic family members are divided into two groups. The multi-region pro-apoptotic proteins, such as Bax, Bak and Bok, are conventionally thought to have BH1-3 regions, whereas the BH3-only proteins are proposed to share homology in the BH3 region only. Members of BH3-only proteins include Bad, Bim, Bid, Noxa, Puma, Bik/Blk, Bmf, Hrk/DP5, Beclin-1 and Mule (Xu G. et al., Bioorg. Med. Chem. 2017, 25, 5548-5556; Hardwick J. M. et al., Cell. 2009, 138, 404; Reed J. C., Cell Death Differ. 2018, 25, 3-6; Kang M. H. et al., Clin Cancer Res 2009, 15, 1126-1132). The pro-apoptotic members (such as BAX and BAK), upon activation, form a homo-oligomer in the outer mitochondrial membrane that leads to pore formation and the escape of mitochondrial contents, a step in triggering apoptosis. Antiapoptotic members of the Bcl-2 family (such as Bcl-2, Bel-XL, and Mcl-1) block the activity of BAX and BAK. In normal cells, this process is tightly regulated. Abnormal cells can dysregulate this process to avoid cell death. One of the ways that cancer cells can accomplish this is by upregulating the antiapoptotic members of the Bcl-2 family of proteins. Overexpression or up-regulation of the anti-apoptotic Bcl-2 family proteins enhance cancer cell survival and cause resistance to a variety of anticancer therapies.

Aberrant expression or function of the proteins responsible for apoptotic signaling contributes to numerous human pathologies including auto-immune diseases, neurodegeneration (such as Parkinson's disease, Alzheimer's disease and ischaemia), inflammatory diseases, viral infections and cancer (such as colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, pancreatic cancer, etc.) (Hanahan D. et al., Cell 2000, 100. 57-70). Herein, it is prospective to target key apoptosis regulators for cancer treatment (Kale J. et al., Cell Death Differ. 2018, 25, 65-80; Vogler M. et al., Cell Death Differ. 2009, 16, 360-367).

By overexpressing one or more of these pro-survival proteins, cancer cells can evade elimination by normal physiological processes and thus gain a survival advantage. Myeloid Cell Leukemia-1 (Mcl-1) is a member of the pro-survival Bcl-2 family of proteins. Mcl-1 has the distinct trait of being essential for embryonic development as well as the survival of all hematopoietic lineages and progenitor populations. Mcl-1 is one of the most common genetic aberrations in human cancer and is highly expressed in many tumor types. Mcl-1 overexpression in human cancers is associated with high tumor grade and poor survival (Beroukhim R. et al., Nature 2010, 463, 899-905). Mcl-1 overexpression prevents cancer cells from undergoing programmed cell death (apoptosis), allowing the cells to survive despite widespread genetic damage. Further, its amplification is associated with both intrinsic and acquired resistance to a wide variety of antitumorigenic agents including chemotherapeutic agents such as microtubule binding agents, paclitaxel and gemcitabine, as well as apoptosis-inducing agents such as TRAIL, the Bcl-2 inhibitor, venetoclax, and the Bcl-2Bcl-XL dual inhibitor navitoclax. Not only do gene silencing approaches that specifically target Mcl-1 circumvent this resistance phenotype, but certain cancer cell types frequently undergo cell death in response to Mcl-1 silencing, indicating a dependence on Mcl-1 for survival. Consequently, approaches that inhibit Mcl-1 function are of considerable interest for cancer therapy (Wertz I. E et al., Nature 2011, 471, 110-114; Zhang B. et al., Blood 2002, 99, 1885-1893).

### SUMMARY

The disclosure is directed to compounds of Formula I:

or a pharmaceutically acceptable salt or solvate thereof;

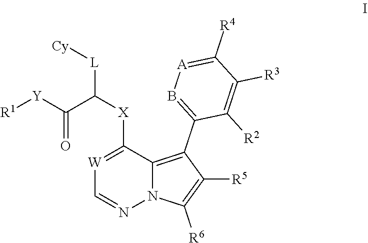

wherein

W is N or CR;

X is —CH₂—, —NH— or —O—;

Y is —NH— or —O—;

A is N, or CR⁷;

B is N, or CR⁸;

L is (CR⁹R¹⁰)ₘ, (CR⁹R¹⁰)ₚO(CR⁹R¹⁰)_{q}, (CR⁹R¹⁰)ₚS(CR⁹R¹⁰)_{q}, or (CR⁹R¹⁰)ₚNR¹¹(CR⁹R¹⁰)_{q};

Cy is aryl, heteroaryl, cycloalkyl, or heterocycloalkyl, each optionally substituted by 1, 2, 3, 4, or 5 R¹²;

R is H, halo, C₁-C₆ alkyl, C₁-C₆ haloalkoxy; C₂-C₆ alkenyl, C₂-C₆ alkynyl, C₁-C₆ haloalkyl, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};

R¹ is H, C₁-C₈ alkyl, aryl, heteroaryl, aryl-C₁-C₆ alkyl, or heteroaryl-C₁-C₆ alkyl, S(O)R^{B}, S(O)NR^{C}R^{D}, S(O)₂R^{B}, or S(O)₂NR^{C}R^{D};

each R², R³, R⁴, R⁷ and R⁸ is independently selected from H, halo, C₁-C₆ alkyl, C₂-C₆ alkenyl, C₂-C₆ alkynyl, C₁-C₆ haloalkyl, OH, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};

or R² and R³, or R³ and R⁴ together with the carbon atom to which they are attached form a 4- to 7-membered cycloalkyl group or 5- to 7-membered heterocycloalkyl group, or heteroaryl group, each optionally substituted by 1, 2, or 3 substituents independently selected from halo, C₁-C₆ alkyl, C₂-C₆ alkenyl, C₂-C₆ alkynyl, C₁-C₆ haloalkyl, —C₁-C₆ alkyl NR^{c}R^{d}, —C₁-C₆ alkyl-Cy¹, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, P(O)R^{e}R^{f}, P(O)OR^{e}OR^{f}, S(O)R^{b}, S(O)NR^{c}R^{d}, S(O)₂R^{b}, NR^{c}S(O)₂R^{b}, or S(O)₂NR^{c}R^{d};

R⁵ is halo, C₁-C₆ alkyl, C₂-C₆ alkenyl, C₂-C₆ alkynyl, C₁-C₆ haloalkyl, CN, C(O)R^{B}, C(O)NR^{C}R^{D}, S(O)R^{B}, S(O)NR^{C}R^{D}, S(O)₂R^{B}, S(O)₂NR^{C}R^{D}, (CR⁹R¹⁰)ₘCy¹, C₂-C₆ alkenyl-Cy¹, C₂-C₆ alkynyl-Cy¹, C₂-C₆ alkynyl-O-Cy¹, (CR⁹R¹⁰)ₚO(CR⁹R¹⁰)_{q}Cy¹, (CR⁹R¹⁰)ₚS(CR⁹R¹⁰)_{q}Cy¹, (CR⁹R¹⁰)ₚNR¹¹(CR⁹R¹⁰)_{q}Cy¹, Cy²(CR⁹R¹⁰)ₘCy¹, Cy²(CR⁹R¹⁰)ₚO(CR⁹R¹⁰)_{q}Cy¹, Cy²(CR⁹R¹⁰)ₚS(CR⁹R¹⁰)_{q}Cy¹, or Cy²(CR⁹R¹⁰)ₚNR¹¹(CR⁹R¹⁰)_{q}Cy¹;

R⁶ is H, C₁-C₆ alkyl, C₂-C₆ alkenyl, C₂-C₆ alkynyl, C₁-C₆ haloalkyl, CN, NO₂, C(O)OR^{A}, C(O)R^{B}, C(O)NR^{C}R^{D}, S(O)R^{B}, S(O)NR^{C}R^{D}, S(O)₂R^{B}, or S(O)₂NR^{C}R^{D};

each R⁹ and R¹⁰ is independently selected from H, halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂;

or R⁹ and R¹⁰ together with the C atom to which they are attached form a 3, 4, 5, 6, or 7-membered cycloalkyl or heterocycloalkyl ring, each optionally substituted by 1, 2, or 3 substituents independently selected from halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂;

R¹¹ is H, C₁-C₆ alkyl, C₂-C₆ alkenyl, or C₂-C₆ alkynyl;

R¹² is H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{D}, P(O)R^{E}R^{F}, P(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, Cy³, OCy³, O—C₁-C₆ alkyl-Cy³, or O—C₁-C₆ alkyl-Cy³-C₀-C₆ alkyl-Cy⁴;

wherein two adjacent R¹², together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl;

Cy¹ is aryl, heteroaryl, cycloalkyl, or heterocycloalkyl, each optionally substituted by 1, 2, 3, 4, or 5 R¹³;

each Cy², Cy³, and Cy⁴ are independently selected from aryl, cycloalkyl, heteroaryl, or heteorcycloalkyl;

R¹³ is H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{D}, P(O)R^{E}R^{F}, P(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, aryl, cycloalkyl, heteroaryl, or heterocycloalkyl;

wherein two adjacent R¹³, together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, halosulfanyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl;

wherein the alkyl, alkenyl, alkynyl, haloalkyl, haloalkoxy and alkoxy groups of any of the R¹, R², R³, R⁴, R⁵, R⁶, R⁷, R⁸, R⁹, R¹⁰, and R¹¹ substituents can be unsubstituted or substituted with 1, 2, or 3 R¹⁴ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, B(OH)₂, OR^{a1}, SR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, P(O)OR^{e}OR^{f}, OP(O)OR^{e}OR^{f}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl;

wherein the aryl, heteroaryl, cycloalkyl and heterocycloalkyl groups, arylalkyl and heteroarylalkyl groups of any of the R¹, R⁹, R¹⁰, Cy², Cy³ and Cy⁴ groups can be unsubstituted or substituted with 1, 2, 3 or 4 R¹⁵ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, B(OH)₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl;

R^{A} is independently selected from H, C₁-C₆ alkyl, C₂-C₄ alkenyl, C₂-C₄ alkynyl, cycloalkyl, cycloalkylalkyl, —C₁-C₆ alkyl-Cy³, heterocycloalkyl, heterocycloalkylalkyl, aryl, or heteroaryl, wherein said C₁-C₆ alkyl, C₂-C₄ alkenyl, C₂-C₄ alkynyl, cycloalkyl, cycloalkylalkyl, heterocycloalkyl, heterocycloalkylalkyl, aryl or heteroaryl is optionally substituted with 1, 2, or 3 substituents independently selected from OH, CN, halo, C₁-C₄ alkyl; NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d};

R^{B} is independently selected from H, C₁-C₆ alkyl, C₂-C₄ alkenyl, C₂-C₄ alkynyl, cycloalkyl, heterocycloalkyl, aryl, or heteroaryl, wherein said C₁-C₆ alkyl, C₂-C₄ alkenyl, C₂-C₄ alkynyl, cycloalkyl, heterocycloalkyl, aryl, or heteroaryl is optionally substituted with 1, 2, or 3 substituents independently selected from OH, CN, amino, alkylamino, dialkylamino, halo, or C₁-C₄ alkyl;

each R^{C} and R^{D} are independently selected from H, C₁-C₆ alkyl, C₂-C₄ alkenyl, C₂-C₄ alkynyl, wherein said C₁-C₆ alkyl, C₂-C₄ alkenyl, C₂-C₄ alkynyl is optionally substituted with 1, 2, or 3 substituents independently selected from OH, CN, amino, alkylamino, dialkylamino, halo, or C₁-C₄ alkyl;

or R^{C} and R^{D} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, or C₁-C₄ alkyl;

R^{E} is independently H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl;

R^{F} is independently H, C₁-C₄ alkyl, C₂-C₄ alkenyl, C₂-C₄ alkynyl, aryl, cycloalkyl, heteroaryl, or heterocycloalkyl;

each R^{a} and R^{a1} are independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy;

each R^{b} and R^{b1} are independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy;

each R^{c} and R^{d} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl, wherein said C₁₋₄ alkyl, C₁₋₄ haloalkyl, C₂₋₄ alkenyl, C₂₋₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, aryl, heteroaryl, C(O)OR^{a1}, C(O)R^{b1}, S(O)₂R^{b1}, alkoxyalkyl, and alkoxyalkoxy;

or R^{c} and R^{d} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, C₁-C₄ haloalkoxy, hydroxyalkyl, cyanoalkyl, aryl, heteroaryl, C(O)OR^{a1}, C(O)R^{b1}, S(O)₂R^{b1}, alkoxyalkyl, and alkoxyalkoxy;

each R^{c1} and R^{d1} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl, wherein said C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy;

or R^{c1} and R^{d1} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, and C₁-C₄ haloalkoxy;

each R^{e} and R^{e1} are independently selected from H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl;

each R^{f} and R^{f1} are independently selected from H, C₁-C₄ alkyl, C₂-C₄ alkenyl, C₂-C₄ alkynyl, aryl, cycloalkyl, heteroaryl, or heterocycloalkyl;

each m is independently 0, 1, 2, 3, or 4;

each p is independently 0, 1, 2, 3, or 4; and

each q is independently 0, 1, 2, 3, or 4.

Stereoisomers of the compounds of Formula I, and the pharmaceutical salts and solvates thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, and the like.

As used herein, “alkynyl” refers to an alkyl group having one or more triple carbon-carbon bonds. Example alkynyl groups include ethynyl, propynyl, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocyclic rings. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like.

In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to a non-aromatic heterocycle where one or more of the ring-forming atoms is a heteroatom such as an O, N, or S atom. Heterocycloalkyl groups can include mono- or polycyclic (e.g., having 2, 3 or 4 fused rings) ring systems as well as spirocyclic rings. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion. Also included in the definition of heterocycloalkyl are moieties where one or more ring-forming atoms is substituted by 1 or 2 oxo or sulfido groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “arylcycloalkyl” refers to cycloalkyl group substituted by an aryl group.

As used herein, “arylheterocycloalkyl” refers to a heterocycloalkyl group substituted by an aryl group.

As used herein, “arylheteroaryl” refers to a heteroaryl group substituted by an aryl group.

As used herein, “biaryl” refers to an aryl group substituted by another aryl group.

As used herein, “heteroarylcycloalkyl” refers to a cycloalkyl group substituted by a heteroaryl group.

As used herein, “heteroarylheterocycloalkyl” refers to a heterocycloalkyl group substituted by a heteroaryl group.

As used herein, “heteroarylaryl” refers to an aryl group substituted by a heteroaryl group.

As used herein, “biheteroaryl” refers to a heteroaryl group substituted by another heteroaryl group.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

As used herein, “cyanoalkyl” refers to an alkyl group substituted by CN.

As used herein, “alkoxyalkyl” refers to an alkyl group substituted by an alkoxy group.

As used herein, “alkoxyalkoxy” refers to an alkoxy group substituted by alkoxy.

As used herein, “haloalkoxy” refers to an —O-(haloalkyl) group.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to alkyl substituted by heteroaryl and “heterocycloalkylalkyl” refers to alkyl substituted by heterocycloalkyl.

As used herein, “amino” refers to NH₂.

As used herein, “alkylamino” refers to an amino group substituted by an alkyl group.

As used herein, “dialkylamino” refers to an amino group substituted by two alkyl groups.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which is was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., enantiomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

The present invention is directed to compounds of Formula I:

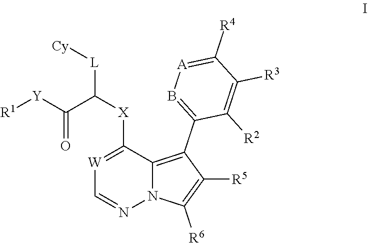

or a pharmaceutically acceptable salt or solvate thereof.

In the compounds of Formula I, W is N or CR. In some embodiments, W is N. In other embodiments, W is CR.

In the compounds of Formula I, X is —CH₂—, —NH— or —O—. In some embodiments, X is —CH₂—. In some embodiments, X is —NH—. In other embodiments, X is —O—.

In the compounds of Formula I, Y is —NH— or —O—. In some embodiments, Y is —NH—. In other embodiments, Y is —O—.

In the compounds of Formula I, A is N or CR⁷. In some embodiments, A is N. In some embodiments, A is CR⁷.

In the compounds of Formula I, B is N or CR⁸. In some embodiments, B is N. In some embodiments, B is CR⁸.

In the compounds of Formula I, L is (CR⁹R¹⁰)ₘ, (CR⁹R¹⁰)ₚO(CR⁹R¹⁰)_{q}, (CR⁹R¹⁰)ₚS(CR⁹R¹⁰)_{q}, or (CR⁹R¹⁰)ₚNR¹¹(CR⁹R¹⁰)_{q}. In some embodiments, L is (CR⁹R¹⁰)ₘ. In some embodiments, L is (CR⁹R¹⁰)ₚO(CR⁹R¹⁰)_{q}. In some embodiments, L is (CR⁹R¹⁰)ₚS(CR⁹R¹⁰)_{q} In some embodments, L is (CR⁹R¹⁰)ₚNR¹¹(CR⁹R¹⁰)_{q}. In some embodiments, L is (CR⁹R¹⁰)ₘ wherein m=1, R⁹ is H, and R¹⁰ is H. Thus, in some embodiments, L is —CH₂—.

In the compounds of Formula I, Cy is aryl, heteroaryl, cycloalkyl, or heterocycloalkyl, each optionally substituted by 1, 2, 3, 4, or 5 R¹². In some embodiments, Cy is aryl, optionally substituted by 1, 2, 3, 4, or 5 R¹². In some embodiments, Cy is heteroaryl, optionally substituted by 1, 2, 3, 4, or 5 R¹². In some embodiments, Cy is cycloalkyl, optionally substituted by 1, 2, 3, 4, or 5 R¹². In some embodiments, Cy is heterocycloalkyl, optionally substituted by 1, 2, 3, 4, or 5 R¹².

In the compounds of Formula I, R is independently selected from H, halo, C₁-C₆ alkyl, C₁-C₆ haloalkoxy; C₂-C₆ alkenyl, C₂-C₆ alkynyl, C₁-C₆ haloalkyl, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D}. In some embodiments, R is H.

In the compounds of Formula I, R¹ is selected from H, C₁-C₈ alkyl, aryl, heteroaryl, aryl-C₁-C₆ alkyl, or heteroaryl-C₁-C₆ alkyl, S(O)R^{B}, S(O)NR^{C}R^{D}, S(O)₂R^{B}, or S(O)₂NR^{C}R^{D}. In some embodiments, R¹ is H. In other embodiments, R¹ is S(O)₂R^{B}. In other embodiments, R¹ is S(O)₂NR^{C}R^{D}. In some embodiments, R¹ is C₁-C₈ alkyl substituted with OC(O)OR^{a1}, OC(O)NR^{c1}R^{d1}, or OP(O)OR^{e}OR^{f}, wherein R^{a1}, R^{c1}, R^{d1}, R^{e}, and R^{f} are each C₁-C₄ alkyl. In some embodiments, R¹ is

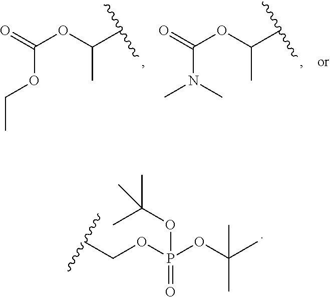

In the compounds of Formula I, each R², R³, R⁴, R⁷ and R⁸ are independently selected from H, halo, C₁-C₆ alkyl, C₂-C₆ alkenyl, C₂-C₆ alkynyl, C₁-C₆ haloalkyl, OH, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D}. In some embodiments, R² is C₁-C₆ alkyl, for example, —CH₃. In some embodiments, R³ is halo, for example —Cl. In some embodiments, R⁴ is —OR^{A}. In some embodiments, R⁷ is H. In some embodiments, R⁸ is H.

In some embodiments wherein R⁴ is —OR^{A}, R^{A} is —C₁-C₆ alkyl-Cy³. In some embodiments, wherein R⁴ is —OR^{A}, R^{A} is —C₁-C₆ alkyl-heterocycloalkyl. In some embodiments, wherein R⁴ is —OR^{A}, R^{A} is

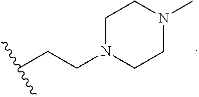 [SMILES: *CCN1CCN(C)CC1]

In other aspects, R² and R³, or R³ and R⁴ together with the carbon atom to which they are attached form a 4- to 7-membered cycloalkyl group or 5- to 7-membered heterocycloalkyl group, or heteroaryl group, each optionally substituted by 1, 2, or 3 substituents independently selected from halo, C₁-C₆ alkyl, C₂-C₆ alkenyl, C₂-C₆ alkynyl, C₁-C₆ haloalkyl, —C₁-C₆ alkyl NR^{c}R^{d}, —C₁-C₆ alkyl-Cy¹, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, P(O)R^{e}R^{f}, P(O)OR^{e}OR^{f}, S(O)R^{b}, S(O)NR^{c}R^{d}, S(O)₂R^{b}, NR^{c}S(O)₂R^{b}, or S(O)₂NR^{c}R^{d}.

In the compounds of Formula I, R⁵ is halo, C₁-C₆ alkyl, C₂-C₆ alkenyl, C₂-C₆ alkynyl, C₁-C₆ haloalkyl, CN, C(O)R^{B}, C(O)NR^{C}R^{D}, S(O)R^{B}, S(O)NR^{C}R^{D}, S(O)₂R^{B}, S(O)₂NR^{C}R^{B}, (CR⁹R¹⁰)ₘCy¹, C₂-C₆ alkenyl-Cy¹, C₂-C₆ alkynyl-Cy¹, C₂-C₆ alkynyl-O-Cy¹, (CR⁹R¹⁰)ₚO(CR⁹R¹⁰)_{q}Cy¹, (CR⁹R¹⁰)ₚS(CR⁹R¹⁰)_{q}Cy¹, (CR⁹R¹⁰)ₚNR¹¹(CR⁹R¹⁰)_{q}Cy¹, Cy²(CR⁹R¹⁰)ₘCy¹, Cy²(CR⁹R¹⁰)ₚO(CR⁹R¹⁰)_{q}Cy¹, Cy²(CR⁹R¹⁰)ₚS(CR⁹R¹⁰)_{q}Cy¹, or Cy²(CR⁹R¹⁰)ₚNR¹¹(CR⁹R¹⁰)_{q}Cy¹. In some embodiments, R⁵ is (CR⁹R¹⁰)ₘCy¹ wherein m=0 and Cy¹ is aryl substituted with R¹³, wherein R¹³ is halo, e.g., —F. In some embodiments, R⁵ is fluorophenyl. In some embodiments, R⁵ is 4-fluorophenyl.

In the compounds of Formula I, R⁶ is H, C₁-C₆ alkyl, C₂-C₆ alkenyl, C₂-C₆ alkynyl, C₁-C₆ haloalkyl, CN, NO₂, C(O)OR^{A}, C(O)R^{B}, C(O)NR^{C}R^{D}, S(O)R^{B}, S(O)NR^{C}R^{D}, S(O)₂R^{B}, or S(O)₂NR^{C}R^{B}. In some embodiments, R⁶ is H.

In the compounds of Formula I, each R⁹ and R¹⁰ is independently selected from H, halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂. In some embodiments, each R⁹ and each R¹⁰ is H.

In some embodiments, R⁹ and R¹⁰ in Formula I, together with the C atom to which they are attached form a 3, 4, 5, 6, or 7-membered cycloalkyl or heterocycloalkyl ring, each optionally substituted by 1, 2, or 3 substituents independently selected from halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂.

In the compounds of Formula I, R¹¹ is H, C₁-C₆ alkyl, C₂-C₆ alkenyl, or C₂-C₆ alkynyl.

In the compounds of Formula I, R¹² is independently selected from H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, P(O)R^{E}R^{F}, P(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, Cy³, OCy³, O—C₁-C₆ alkyl-Cy³, or O—C₁-C₆ alkyl-Cy³-C₀-C₆ alkyl-Cy⁴; wherein two adjacent R¹², together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{C}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl. In some embodiments, R¹² is OR^{A}. In some embodiments, R¹² is OR^{A} where R^{A} is C₁-C₆ alkyl substituted by 3 fluorine atoms. Thus, in some embodiments, R¹² is —OCH₂CF₃.

In some embodiments, R¹² is O—C₁-C₆ alkyl-Cy³. In other embodiments, R¹² is O—C₁-C₆ alkyl-Cy³ where Cy³ is cycloalkyl. Thus, in some embodiments, R¹² is —O—CH₂-cycloalkyl. In other embodiments, R¹² is —O—CH₂-cyclopropyl.

In other embodiments, R¹² is O—C₁-C₆ alkyl-Cy³ wherein Cy³ is heteroaryl substituted by R¹⁵. In other embodiments, R¹² is O—C₁-C₆ alkyl-Cy³ wherein Cy³ is heteroaryl substituted by R¹⁵ wherein R¹⁵ is C₁-C₆ haloalkyl. In some embodiments, R¹² is

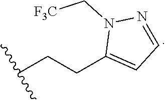 [SMILES: *CCc1ccnn1CC(F)(F)F]

In some embodiments, R¹² is O—C₁-C₆ alkyl-Cy³-C₀-C₆ alkyl-Cy⁴. In some embodiments, R¹² is O—C₁-C₆ alkyl-Cy³-C₀-C₆ alkyl-Cy⁴ wherein Cy³ is heteroaryl and Cy⁴ is aryl. In some embodiments, R¹² is O—C₁-C₆ alkyl-Cy³-C₀-C₆ alkyl-Cy⁴ wherein Cy³ is pyrimidinyl and Cy⁴ is phenyl. In some embodiments, R¹² is O—C₁-alkyl-Cy³-C₀ alkyl-Cy⁴ wherein Cy³ is pyrimidinyl and Cy⁴ is phenyl substituted by R¹⁵. In some embodiments, R¹² is O—C₁-alkyl-Cy³-C₀ alkyl-Cy⁴ wherein Cy³ is pyrimidinyl and Cy⁴ is phenyl by R¹⁵ wherein R¹⁵ is C₁-C₆ haloalkyl, or —OR^{a1} wherein R^{a1} is C₁-C₄ alkyl. In some embodiments, R¹² is

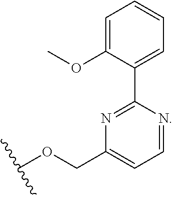 [SMILES: *OCc1ccnc(-c2ccccc2OC)n1]

In the compounds of Formula I, Cy¹ is aryl, heteroaryl, cycloalkyl, or heterocycloalkyl, each optionally substituted by 1, 2, 3, 4, or 5 R¹³.

In the compounds of Formula I, each Cy², Cy³, and Cy⁴ is independently selected from aryl, cycloalkyl, heteroaryl, or heteorcycloalkyl. In some embodiments, Cy³ is heteroaryl. In some embodiments, Cy³ is pyrimidinyl. In other embodiments, Cy³ is pyrazolyl. In other embodiments, Cy³ is heterocycloalkyl. In some embodiments, Cy³ is 4-methylpiperazin-1-yl.

In compounds of Formula I, R¹³ is independently selected from H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, P(O)R^{E}R^{F}, P(O)OR^{E}OR^{F}, S(O)R^{B}, S(O)NR^{C}R^{B}, S(O)₂R^{B}, NR^{C}S(O)₂R^{B}, S(O)₂NR^{C}R^{B}, aryl, cycloalkyl, heteroaryl, or heterocycloalkyl; wherein two adjacent R¹³, together with the atoms to which they are attached, optionally form a fused 4-10 membered cycloalkyl ring or a fused 4-10 membered heterocycloalkyl ring; each optionally substituted by 1, 2, or 3 substituents independently selected from halo, C₁-C₆ alkyl, C₂-C₆ alkenyl, C₂-C₆ alkynyl, C₁-C₆ haloalkyl, halosulfanyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{C}C(O)NR^{c}R^{d}, NR^{c}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl.

In compounds of Formula I, the alkyl, alkenyl, alkynyl, haloalkyl, haloalkoxy and alkoxy groups of any of the R¹, R², R³, R⁴, R⁵, R⁶, R⁷, R⁸, R⁹, R¹⁰, and R¹¹ substituents can be unsubstituted or substituted with 1, 2, or 3 R¹⁴ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, B(OH)₂, OR^{a1}, SR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1}, NR^{c1}R^{d1}, NR^{c1}C(O)R^{b1}, NR_{c1}C(O)NR_{c1}R^{d1}, NR^{c1}C(O)OR^{a1}, P(O)OR^{e}OR^{f}, OP(O)OR^{e}OR^{f}, S(O)R^{b1}, S(O)NR^{c1}R^{d1}, S(O)₂R^{b1}, NR^{c1}S(O)₂R_{b1}, S(O)₂NR^{c1}R^{d1}, aryl, heteroaryl, cycloalkyl, or heterocycloalkyl; wherein the aryl, heteroaryl, cycloalkyl and heterocycloalkyl groups, arylalkyl and heteroarylalkyl groups of any of the R¹, R⁹, R¹⁰, Cy², Cy³ and Cy⁴ groups can be unsubstituted or substituted with 1, 2, 3 or 4 R¹⁵ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, B(OH)₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl.

In compounds of Formula I, the aryl, heteroaryl, cycloalkyl and heterocycloalkyl groups, arylalkyl and heteroarylalkyl groups of any of the R¹, R⁹, R¹⁰, Cy², Cy³ and Cy⁴ groups can be unsubstituted or substituted with 1, 2, 3 or 4 R¹⁵ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, B(OH)₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl.

In compounds of Formula I, R^{A} is independently selected from H, C₁-C₆ alkyl, C₂-C₄ alkenyl, C₂-C₄ alkynyl, cycloalkyl, cycloalkylalkyl, —C₁-C₆ alkyl-Cy³, heterocycloalkyl, heterocycloalkylalkyl, aryl, or heteroaryl, wherein said C₁-C₆ alkyl, C₂-C₄ alkenyl, C₂-C₄ alkynyl, cycloalkyl, cycloalkylalkyl, heterocycloalkyl, heterocycloalkylalkyl, aryl or heteroaryl is optionally substituted with 1, 2, or 3 substituents independently selected from OH, CN, halo, C₁-C₄ alkyl; NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}.

In some embodiments, R^{A} is independently selected from H, C₁-C₆ alkyl, C₂-C₄ alkenyl, C₂-C₄ alkynyl, cycloalkyl, heterocycloalkyl, aryl, heteroaryl, or C₁-C₆ alkyl-Cy³ wherein said C₁-C₆ alkyl, C₂-C₄ alkenyl, C₂-C₄ alkynyl, cycloalkyl, heterocycloalkyl, aryl or heteroaryl is optionally substituted with 1, 2, or 3 substituents independently selected from OH, CN, amino, halo, C₁-C₄ haloalkyl or C₁-C₄ alkyl.

In compounds of Formula I, R^{B} is independently selected from H, C₁-C₆ alkyl, C₂-C₄ alkenyl, C₂-C₄ alkynyl, cycloalkyl, heterocycloalkyl, aryl, or heteroaryl, wherein said C₁-C₆ alkyl, C₂-C₄ alkenyl, C₂-C₄ alkynyl, cycloalkyl, heterocycloalkyl, aryl, or heteroaryl is optionally substituted with 1, 2, or 3 substituents independently selected from OH, CN, amino, alkylamino, dialkylamino, halo, or C₁-C₄ alkyl.

In compounds of Formula I, each R^{C} and R^{D} is independently selected from H, C₁-C₆ alkyl, C₂-C₄ alkenyl, C₂-C₄ alkynyl, wherein said C₁-C₆ alkyl, C₂-C₄ alkenyl, C₂-C₄ alkynyl is optionally substituted with 1, 2, or 3 substituents independently selected from OH, CN, amino, alkylamino, dialkylamino, halo, or C₁-C₄ alkyl; or R^{C} and R^{D} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, or C₁-C₄ alkyl.

In compounds of Formula I, R^{E} is independently H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl.

In compound of Formula I, R^{F} is independently H, C₁-C₄ alkyl, C₂-C₄ alkenyl, C₂-C₄ alkynyl, aryl, cycloalkyl, heteroaryl, or heterocycloalkyl.

In compounds of Formula I, each R^{a} and R^{a1} is independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy.

In compounds of Formula I, each R^{b} and R^{b1} is independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy.

In compounds of Formula I, each R^{c} and R^{d} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl, wherein said C₁₋₄ alkyl, C₁₋₄ haloalkyl, C₂₋₄ alkenyl, C₂₋₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, aryl, heteroaryl, C(O)OR^{a1}, C(O)R^{b1}, S(O)₂R^{b1}, alkoxyalkyl, and alkoxyalkoxy; or R^{c} and R^{d} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, C₁-C₄ haloalkoxy, hydroxyalkyl, cyanoalkyl, aryl, heteroaryl, C(O)OR^{a1}, C(O)R^{b1}, S(O)₂R^{b1}, alkoxyalkyl, and alkoxyalkoxy.

In compounds of Formula I, each R^{c1} and R^{d1} is independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl, wherein said C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy; or R^{c1} and R^{d1} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, and C₁₋₄ haloalkoxy.

In compounds of Formula I, each R^{e} and R^{e1} is independently selected from H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl;

In compounds of Formula I, each R^{f} and R^{f1} is independently selected from H, C₁-C₄ alkyl, C₂-C₄ alkenyl, C₂-C₄ alkynyl, aryl, cycloalkyl, heteroaryl, or heterocycloalkyl;

In compounds of Formula I, each m is independently 0, 1, 2, 3, or 4.

In compounds of Formula I, each p is independently 0, 1, 2, 3, or 4.

In compounds of Formula I, each q is 0, 1, 2, 3, or 4.

In some aspects, the present invention is directed to compounds of Formula I-A:

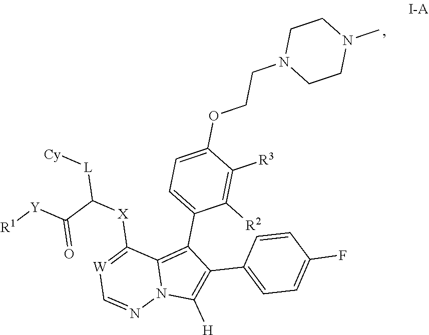

or a pharmaceutically acceptable salt or solvate thereof, wherein
W is N or CH;
X is NH or O;
Y is —NH— or —O—;
L is (—CH₂—)ₘ where m=0, 1, 2, 3, or 4;
Cy is aryl optionally substituted by 1, 2, 3, 4, or 5 R¹²;
R² is C₁-C₆ alkyl;
R³ is halo;
R¹² is OR^{A} where R^{A} is C₁-C₆ alkyl or C₁-C₆ alkyl substituted by 3 fluorine atoms; O—C₁-C₆ alkyl-Cy³ where Cy³ is cycloalkyl; O—C₁-C₆ alkyl-Cy³ where Cy³ is heteroaryl substituted by R¹⁵; or O—C₁-alkyl-Cy³-C₀ alkyl-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl;
R¹ is H, C₁-C₈ alkyl substituted with OC(O)OR^{a1}, OC(O)NR^{c1}R^{d1}, or OP(O)OR^{e}OR^{f}, S(O)₂R^{B}, or S(O)₂NR^{C}R^{D};
R^{B} is C₁-C₆ alkyl or cycloalkyl;
R^{C} and R^{D} are each independently C₁-C₆ alkyl; and
R^{a1}, R^{c1}, R^{d1}, R^{e}, and R^{f} are each C₁-C₄ alkyl.

In some aspects, the present invention is directed to compounds of Formula I-A-1:

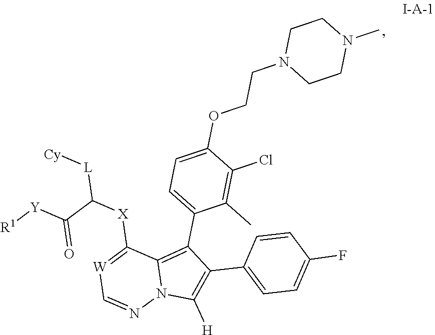

or a pharmaceutically acceptable salt or solvate thereof, wherein
W is N or CH;
X is NH or O;
Y is —NH— or —O—;
L is (—CH₂—)ₘ where m=0, 1, 2, 3, or 4;
Cy is aryl optionally substituted by 1, 2, 3, 4, or 5 R¹²;
R¹² is OR^{A} wherein R^{A} is C₁-C₆ alkyl or C₁-C₆ alkyl substituted by 3 fluorine atoms; O—C₁-C₆ alkyl-Cy³ wherein Cy³ is cycloalkyl; O—C₁-C₆ alkyl-Cy³ wherein Cy³ is heteroaryl substituted by R¹⁵; or O—C₁-alkyl-Cy³-C₀ alkyl-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl;
R¹ is H,

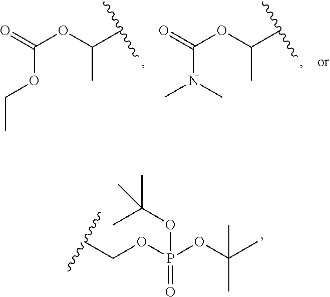

S(O)₂R^{B}, or S(O)₂NR^{C}R^{D};
R^{B} is C₁-C₆ alkyl or cycloalkyl; and
R^{C} and R^{D} are each independently C₁-C₆ alkyl.

In some aspects, the present invention is directed to compounds of Formula I-A-2:

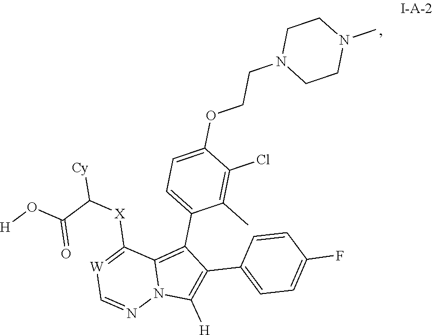

or a pharmaceutically acceptable salt or solvate thereof, wherein
W is N or CH;
X is NH or O;
Cy is aryl optionally substituted by 1, 2, 3, 4, or 5 R¹²;
R¹² is OR^{A} where R^{A} is C₁-C₆ alkyl or C₁-C₆ alkyl substituted by 3 fluorine atoms; O—C₁-C₆ alkyl-Cy³ where Cy³ is cycloalkyl; O—C₁-C₆ alkyl-Cy³ wherein Cy³ is heteroaryl substituted by R¹⁵, or O—C₁-alkyl-Cy³-C₀ alkyl-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl;

In some embodiments of the compound of Formula I-A-2, W is N.

In other embodiments of the compound of Formula I-A-2, W is CH.

In some aspects, the present invention is directed to compounds of Formula I-A-3:

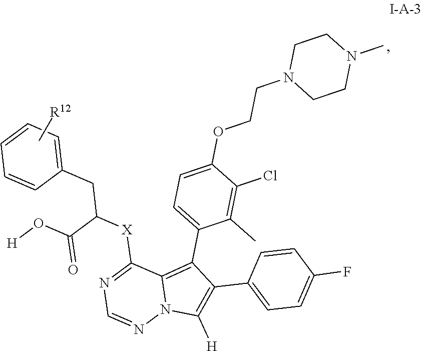

or a pharmaceutically acceptable salt or solvate thereof, wherein
X is NH or O;
R¹² is OR^{A} where R^{A} is C₁-C₆ alkyl substituted by 3 fluorine atoms; O—C₁-C₆ alkyl-Cy³ where Cy³ is cycloalkyl; O—C₁-C₆ alkyl-Cy³ where Cy³ is heteroaryl substituted by R¹⁵; or O—C₁-alkyl-Cy³-C₀ alkyl-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl.

In some embodiments of the compound of Formula I-A-3, W is N.

In other embodiments of the compound of Formula I-A-3, W is CH.

In some aspects, the present invention is directed to compounds of Formula I-A-4:

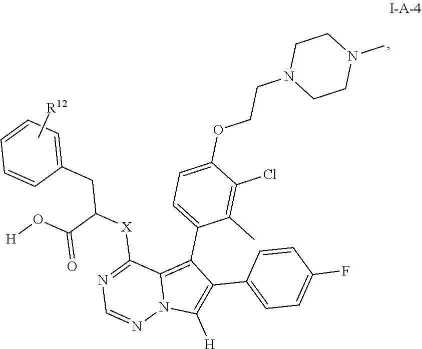

or a pharmaceutically acceptable salt or solvate thereof, wherein
X is NH or O;
R¹² is OR^{A} where R^{A} is C₁-C₆ alkyl substituted by 3 fluorine atoms; O—CH₂-cycloalkyl; O C₁-C₆ alkyl-Cy³ where Cy³ is heteroaryl substituted by R¹⁵, or —O—CH₂-Cy³-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl.

In some embodiments of the compound of Formula I-A-4, W is N.

In other embodiments of the compound of Formula I-A-4, W is CH.

In some aspects, the present invention is directed to compounds of Formula I-A-5:

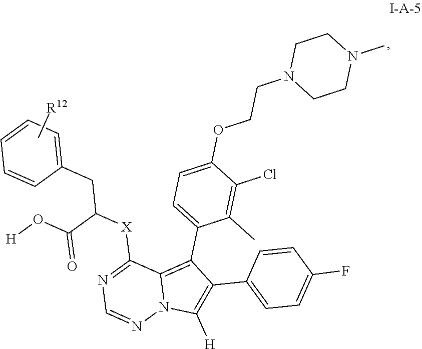

or a pharmaceutically acceptable salt or solvate thereof, wherein
X is NH or O; and
R¹² is —OCH₂CF₃; —O—CH₂-cyclopropyl,

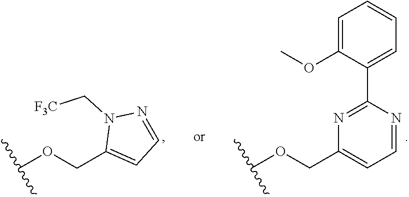 [SMILES: *OCc1ccnn1CC(F)(F)F ; *OCc1ccnc(-c2ccccc2OC)n1]

In some embodiments of the compound of Formula I-A-5, W is N.

In other embodiments of the compound of Formula I-A-5, W is CH.

In some aspects, the present invention is directed to compounds of Formula I-A-6:

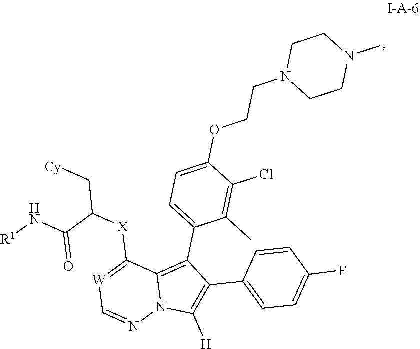

or a pharmaceutically acceptable salt or solvate thereof, wherein
W is N or CH;
X is NH or O;
Cy is aryl optionally substituted by 1, 2, 3, 4, or 5 R¹²;
R¹ is S(O)₂R^{B}, or S(O)₂NR^{C}R^{D};
R^{B} is C₁-C₆ alkyl or cycloalkyl;
R^{C} and R^{D} are each independently C₁-C₆ alkyl;
R¹² is OR^{A} wherein R^{A} is C₁-C₆ alkyl or C₁-C₆ alkyl substituted by 3 fluorine atoms; O—C₁-C₆ alkyl-Cy³ wherein Cy³ is cycloalkyl; O—C₁-C₆ alkyl-Cy³ wherein Cy³ is heteroaryl substituted by R¹⁵; or O—C₁-alkyl-Cy³-C₀ alkyl-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl.

In some embodiments of the compound of Formula I-A-6, W is N.

In other embodiments of the compound of Formula I-A-6, W is CH.

In some aspects, the present invention is directed to compounds of Formula I-A-7:

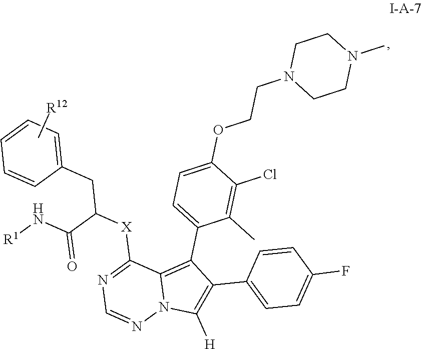

or a pharmaceutically acceptable salt or solvate thereof, wherein
X is NH or O;
R¹ is S(O)₂R^{B}, or S(O)₂NR^{C}R^{D};
R^{B} is methy, isopropyl, or cyclopropyl;
R^{C} and R^{D} are each methyl;
R¹² is OR^{A} wherein R^{A} is C₁-C₆ alkyl substituted by 3 fluorine atoms; O—C₁-C₆ alkyl-Cy³ wherein Cy³ is cycloalkyl; O—C₁-C₆ alkyl-Cy³ wherein Cy³ is heteroaryl substituted
by R¹⁵; or O—C₁-alkyl-Cy³-C₀ alkyl-Cy⁴ wherein Cy³ is pyrimidinyl and Cy⁴ is phenyl substituted by R¹⁵; R¹⁵ is C₁-C₆ haloalkyl, or OR^{a1} wherein R^{a1} is C₁-C₄ alkyl.

In some embodiments of the compound of Formula I-A-7, W is N.

In other embodiments of the compound of Formula I-A-7, W is CH.

In some aspects, the present invention is directed to compounds of Formula I-A-8:

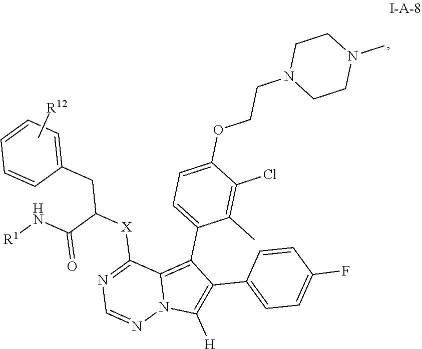

or a pharmaceutically acceptable salt or solvate thereof, wherein
X is NH or O;
R¹ is S(O)₂R^{B}, or S(O)₂NR^{C}R^{D};
R^{B} is methy, isopropyl, or cyclopropyl;
R^{C} and R^{D} are each methyl;
R¹² is OR^{A} where R^{A} is C₁-C₆ alkyl substituted by 3 fluorine atoms; O—CH₂-cycloalkyl; O—C₁-C₆ alkyl-Cy³ where Cy³ is heteroaryl substituted by R¹⁵, or —O—CH₂-Cy³-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl.

In some embodiments of the compound of Formula I-A-8, W is N.

In other embodiments of the compound of Formula I-A-8, W is CH.

In some aspects, the present invention is directed to compounds of Formula I-A-9:

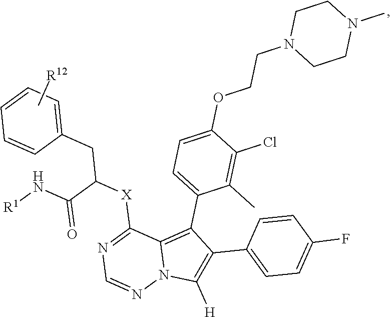

or a pharmaceutically acceptable salt or solvate thereof, wherein
X is NH or O;
R¹ is S(O)₂R^{B}, or S(O)₂NR^{C}R^{D};
R^{B} is methy, isopropyl, or cyclopropyl;
R^{C} and R^{D} are each methyl;
R¹² is —OCH₂CF₃; —O—CH₂-cyclopropyl,

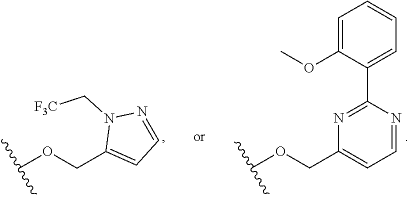 [SMILES: *OCc1ccnn1CC(F)(F)F ; *OCc1ccnc(-c2ccccc2OC)n1]

In some embodiments of the compound of Formula I-A-9, W is N.

In other embodiments of the compound of Formula I-A-9, W is CH.

In some aspects, the present invention is directed to compounds of Formula I-A-10:

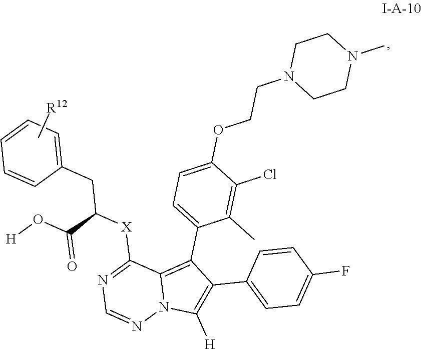

or a pharmaceutically acceptable salt or solvate thereof,

wherein R¹² is OR^{A} where R^{A} is C₁-C₆ alkyl substituted by 3 fluorine atoms.

In some aspects, the present invention is directed to compounds of Formula I-A-11:

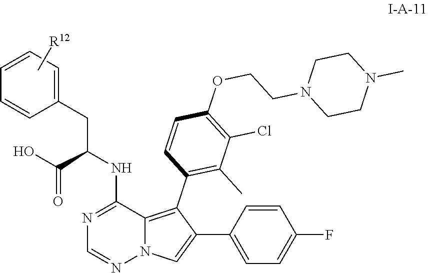

or a pharmaceutically acceptable salt or solvate thereof,

wherein R¹² is OR^{A} where R^{A} is C₁-C₆ alkyl substituted by 3 fluorine atoms.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used.

An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25‰, 50%), 100‰, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%> of a maximum effect in vivo.

In some embodiments, the subject methods utilize a MCL-1 inhibitor with an IC₅₀ value of about or less than a predetermined value, as ascertained in an in vitro assay. In some embodiments, the MCL-1 inhibitor inhibits MCL-1 a with an IC₅₀ value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the MCL-1 inhibitor selectively inhibits MCL-1 a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other MCL-1s.

In some embodiments, the MCL-1 inhibitor selectively inhibits MCL-1 a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other MCL-1s.

The subject methods are useful for treating a disease condition associated with MCL-1. Any disease condition that results directly or indirectly from an abnormal activity or expression level of MCL-1 can be an intended disease condition.

Different disease conditions associated with MCL-1 have been reported. MCL-1 has been implicated, for example, auto-immune diseases, neurodegeneration (such as Parkinson's disease, Alzheimer's disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound of the invention can be administered in combination with a corticosteroid such as triamcinolone, dexamethasone, fluocinolone, cortisone, prednisolone, or flumetholone.

For treating autoimmune or inflammatory conditions, the compound of the invention can be administered in combination with an immune suppressant such as fluocinolone acetonide (Retisert®), rimexolone (AL-2178, Vexol, Alcon), or cyclosporine (Restasis®).

Compounds of the disclosure also include, for example, the compounds identified in Table A.

TABLE A

| Ex. | | | |
|---|---|---|---|
| No. | Structure | MW | Name |
| 1 | 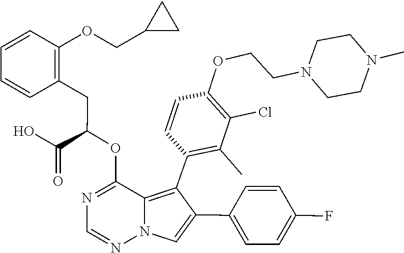 | 714.24 | (2R)-2-[(5Sₐ)-5-[3-chloro-2-methyl-4- [2-(4-methylpiperazin-1- yl)ethoxy]phenyl]-6-(4- fluorophenyl)pyrrolo[2,1-f] [1,2,4]triazin-4-yl]oxy-3-[2- (cyclopropylmethoxy)phenyl]propanoic acid |
| 2 | 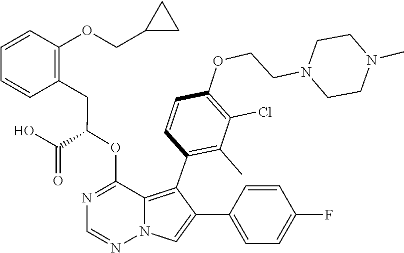 | 714.24 | (2S)-2-[(5Rₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]oxy-3-[2- (cyclopropylmethoxy)phenyl]propanoic acid |
| 3 | 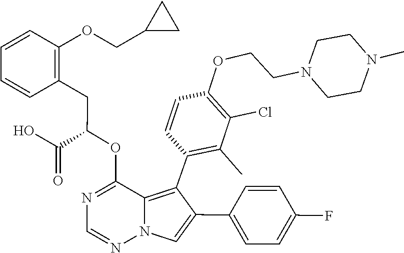 | | (2S)-2-[(5Sₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]oxy-3-[2- (cyclopropylmethoxy)phenyl]propanoic acid |
| 4 | 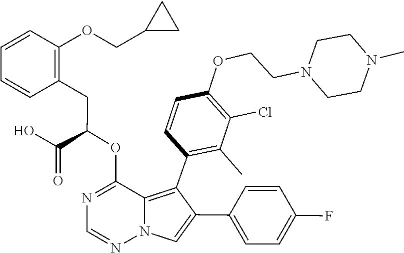 | | (2R)-2-[(5Rₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]oxy-3-[2- (cyclopropylmethoxy)phenyl]propanoic acid |
| 5 | 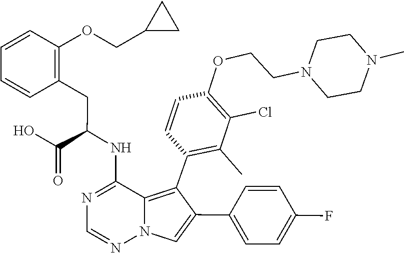 | 713.25 | (2R)-2-{[(5Sₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]amino}-3-[2- (cyclopropylmethoxy)phenyl] propanoic acid |
| 6 | 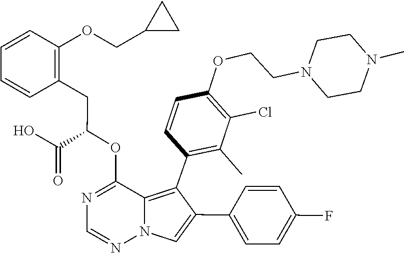 | 713.25 | (2S)-2-{[(5Rₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]amino}-3-[2- (cyclopropylmethoxy)phenyl] propanoic acid |
| 7 | 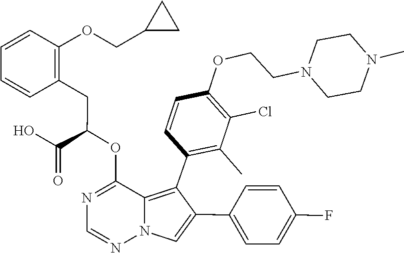 | 713.25 | (2R)-2-{[(5Rₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]amino}-3-[2- (cyclopropylmethoxy)phenyl] propanoic acid |
| 8 | 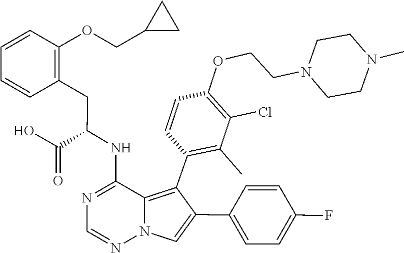 | 713.25 | (2S)-2-{[(5Sₐ)-5-[3-chloro-2-methyl-4- [2-(4-methylpiperazin-1- yl)ethoxy]phenyl]-6-(4- fluorophenyl)pyrrolo[2,1- f][1,2,4]triazin-4-yl]amino}-3-[2- (cyclopropylmethoxy)phenyl] propanoic acid |
| 9 | 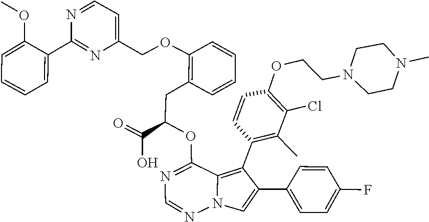 | 858.37 | (2R)-2-{[(5Sₐ)-5-{3-chloro-2-methyl-4- [2-(4-methylpiperazin-1- yl)ethoxy]phenyl}-6-(4- fluorophenyl)pyrrolo[2,1- f][1,2,4]triazin-4-yl]oxy}-3-[2-{[2-(2- methoxyphenyl)pyrimidin-4- yl]methoxy}phenyl)propanoic acid |
| 10 | 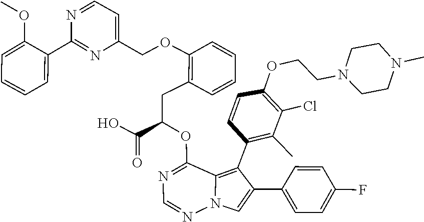 | 858.37 | (2R)-2-{[(5Rₐ)-5-{3-chloro-2-methyl- 4-[2-(4-methylpiperazin-l- yl)ethoxy]phenyl}-6-(4- fluorophenyl)pyrrolo[2,1- f][1,2,4]triazin-4-yl]oxy}-3-[2-{[2-(2- methoxyphenyl)pyrimidin-4- yl]methoxy}phenyl)propanoic acid |
| 11 | 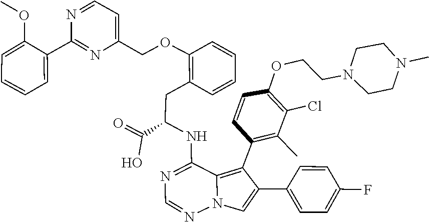 And | 857.38 | (2S)-2-{[(5Rₐ)-5-{3-chloro-2-methyl-4- [2-(4-methylpiperazin-1- yl)ethoxy]phenyl}-6-(4- fluorophenyl)pyrrolo[2,1- f][1,2,4]triazin-4-yl]amino}-3-(2-{[2- (2-methoxyphenyl)pyrimidin-4- yl]methoxy}phenyl)propanoic acid and (2R)-2-{[(5Sₐ)-5-{3-chloro-2-methyl-4- [2-(4-methylpiperazin-1- yl)ethoxy]phenyl}-6-(4- fluorophenyl)pyrrolo[2,1- f][1,2,4]triazin-4-yl]amino}-3-(2-{[2- (2-methoxyphenyl)pyrimidin-4- yl]methoxy}phenyl)propanoic acid |
| | 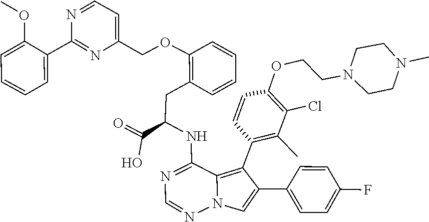 | | |
| 12 | 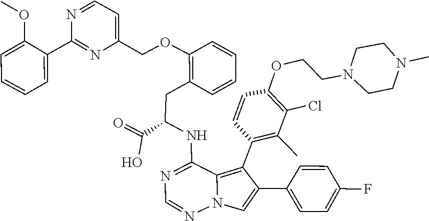 | 857.38 | (2S)-2-{[(5Sₐ)-5-{3-chloro-2-methyl-4- [2-(4-methylpiperazin-1- yl)ethoxy]phenyl}-6-(4- fluorophenyl)pyrrolo[2,1- f][1,2,4]triazin-4-yl]amino}-3-(2-{[2- (2-methoxyphenyl)pyrimidin-4- yl]methoxy}phenyl)propanoic acid |
| 13 | 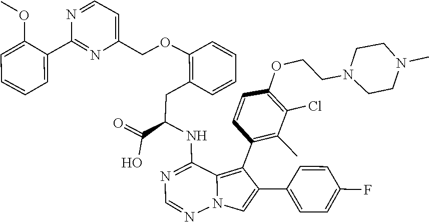 | 857.38 | (2R)-2-{[(5Rₐ)-5-{3-chloro-2-methyl- 4-[2-(4-methylpiperazin-1- yl)ethoxy]phenyl}-6-(4- fluorophenyl)pyrrolo[2,1- f][1,2,4]triazin-4-yl]amino}-3-(2-{[2- (2-methoxyphenyl)pyrimidin-4- yl]methoxy}phenyl)propanoic acid |
| 14 | 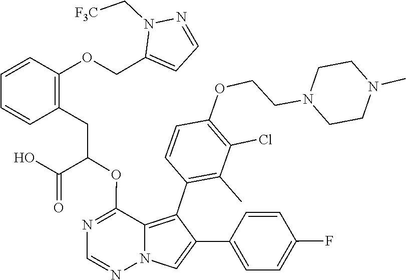 [SMILES: Cc1c(-c2c(-c3ccc(F)cc3)cn3ncnc(OC(Cc4ccccc4OCc4ccnn4CC(F)(F)F)C(=O)O)c23)ccc(OCCN2CCN(C)CC2)c1Cl] | 822.26 | 2-((5-(3-chloro-2-methyl-4-(2-(4- methylpiperazin-l-yl)ethoxy)phenyl)- 6-(4-fluorophenyl)pyrrolo[2,1- f][1,2,4]triazin-4-yl)oxy)-3-(2-((1- (2,2,2-trifluoroethyl)-1H-pyrazol-5- yl)methoxy)phenyl)propanoic acid |
| 15 | 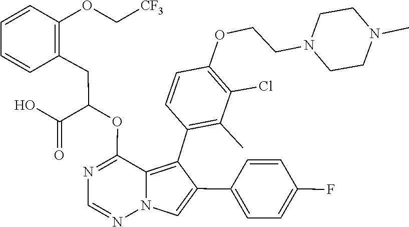 [SMILES: Cc1c(-c2c(-c3ccc(F)cc3)cn3ncnc(OC(Cc4ccccc4OCC(F)(F)F)C(=O)O)c23)ccc(OCCN2CCN(C)CC2)c1Cl] | 742.17 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-3-(2-(2,2,2- trifluoroethoxy)phenyl)propanoic acid |
| 16 | 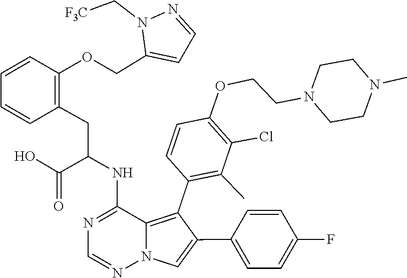 [SMILES: Cc1c(-c2c(-c3ccc(F)cc3)cn3ncnc(NC(Cc4ccccc4OCc4ccnn4CC(F)(F)F)C(=O)O)c23)ccc(OCCN2CCN(C)CC2)c1Cl] | 821.27 | 2-((5-(3-chloro-2-methyl-4-(2-(4- methylpiperazin-l-yl)ethoxy)phenyl)- 6-(4-fluorophenyl)pyrrolo[2,1- f][1,2,4]triazin-4-yl)amino)-3-(2-((1- (2,2,2-trifluoroethyl)-1H-pyrazol-5- yl)methoxy)phenyl)propanoic acid |
| 17 | 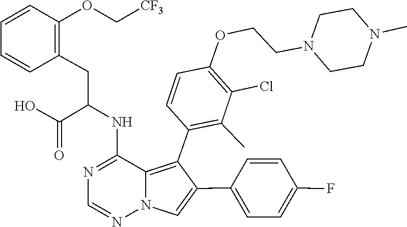 [SMILES: Cc1c(-c2c(-c3ccc(F)cc3)cn3ncnc(NC(Cc4ccccc4OCC(F)(F)F)C(=O)O)c23)ccc(OCCN2CCN(C)CC2)c1Cl] | 741.18 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)amino)-3-(2-(2,2,2- trifluoroethoxy)phenyl)propanoic acid |
| 18 | 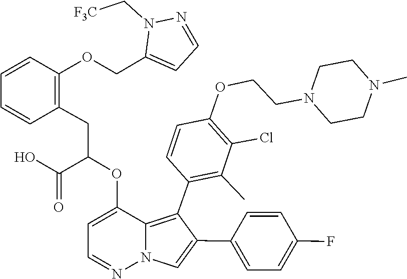 [SMILES: Cc1c(-c2c(-c3ccc(F)cc3)cn3nccc(OC(Cc4ccccc4OCc4ccnn4CC(F)(F)F)C(=O)O)c23)ccc(OCCN2CCN(C)CC2)c1Cl] | 821.27 | 2-((5-(3-chloro-2-methyl-4-(2-(4- methylpiperazin-1-yl)ethoxy)phenyl)- 6-(4-fluorophenyl)pyrrolo[1,2- b]pyridazin-4-yl)oxy)-3-(2-((1-(2,2,2- trifluoroethyl)-1H-pyrazol-5- yl)methoxy)phenyl)propanoic acid |
| 19 | 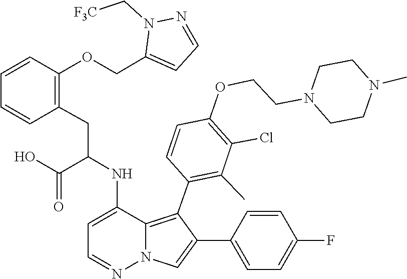 [SMILES: Cc1c(-c2c(-c3ccc(F)cc3)cn3nccc(NC(Cc4ccccc4OCc4ccnn4CC(F)(F)F)C(=O)O)c23)ccc(OCCN2CCN(C)CC2)c1Cl] | 820.29 | 2-((5-(3-chloro-2-methyl-4-(2-(4- methylpiperazin-1-yl)ethoxy)phenyl)- 6-(4-fluorophenyl)pyrrolo[1,2- b]pyridazin-4-yl)amino)-3-(2-((1- (2,2,2-trifluoroethyl)-1H-pyrazol-5- yl)methoxy)phenyl)propanoic acid |
| 20 | 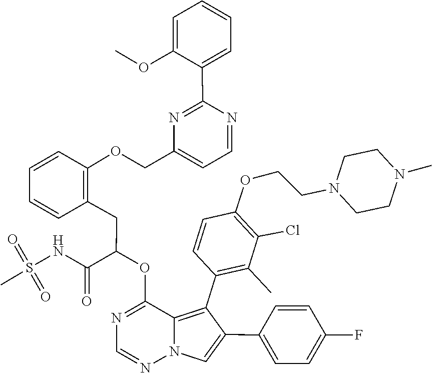 | 935.47 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-3-(2-((2-(2- methoxyphenyl)pyrimidin-4- yl)methoxy)phenyl)-N- (methylsulfonyl)propanamide |
| 21 | 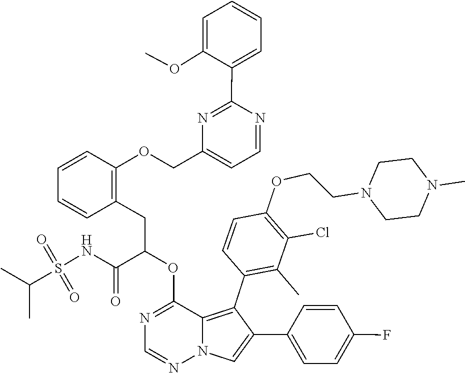 [SMILES: COc1ccccc1-c1nccc(COc2ccccc2CC(Oc2ncnn3cc(-c4ccc(F)cc4)c(-c4ccc(OCCN5CCN(C)CC5)c(Cl)c4C)c23)C(=O)NS(=O)(=O)C(C)C)n1] | 963.52 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-N- (isopropylsulfonyl)-3-(2-((2-(2- methoxyphenyl)pyrimidin-4- yl)methoxy)phenyl)propanamide |
| 22 | 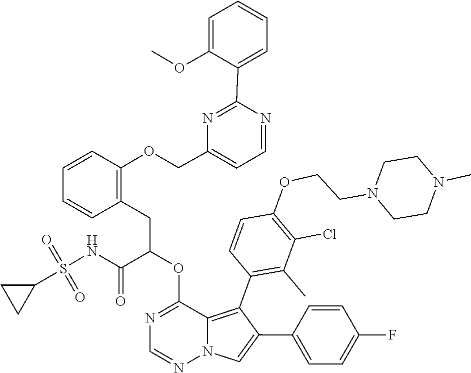 [SMILES: COc1ccccc1-c1nccc(COc2ccccc2CC(Oc2ncnn3cc(-c4ccc(F)cc4)c(-c4ccc(OCCN5CCN(C)CC5)c(Cl)c4C)c23)C(=O)NS(=O)(=O)C2CC2)n1] | 961.51 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-N- (cyclopropylsulfonyl)-3-(2-((2-(2- methoxyphenyl)pyrimidin-4- yl)methoxy)phenyl)propanamide |
| 23 | 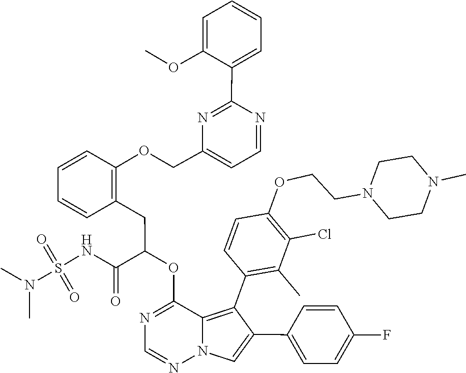 | 964.51 | 2-((5-(3-chloro-2-methyl-4-(2-(4- methylpiperazin-l-yl)ethoxy)phenyl)- 6-(4-fluorophenyl)pyrrolo[2,1- f][1,2,4]triazin-4-yl)oxy)-N-(N,N- dimethylsulfamoyl)-3-(2-[2-(2- methoxyphenyl)pyrimidin-4- yl)methoxy)phenyl)propanamide |
| 24 | 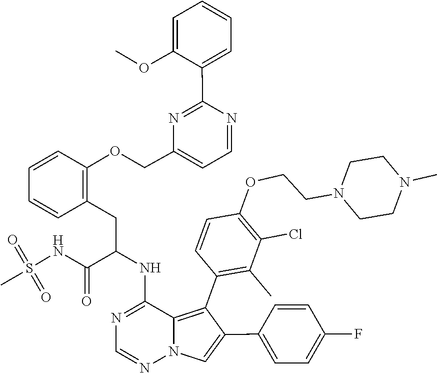 | 934.49 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)amino)-3-(2-((2-(2- methoxyphenyl)pyrimidin-4- yl)methoxy)phenyl)-N- (methylsulfonyl)propanamide |
| 25 | 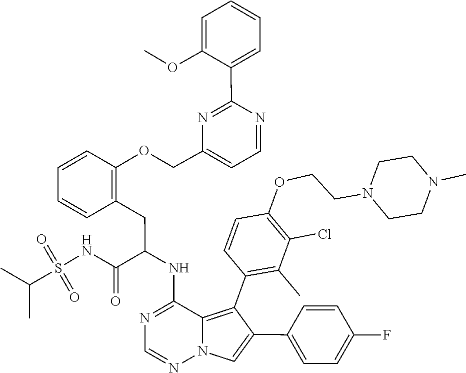 [SMILES: COc1ccccc1-c1nccc(COc2ccccc2CC(Nc2ncnn3cc(-c4ccc(F)cc4)c(-c4ccc(OCCN5CCN(C)CC5)c(Cl)c4C)c23)C(=O)NS(=O)(=O)C(C)C)n1] | 962.54 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)amino)-N- (isopropylsulfonyl)-3-(2-((2-[2- methoxyphenyl)pyrimidin-4- yl)methoxy)phenyl)propanamide |
| 26 | 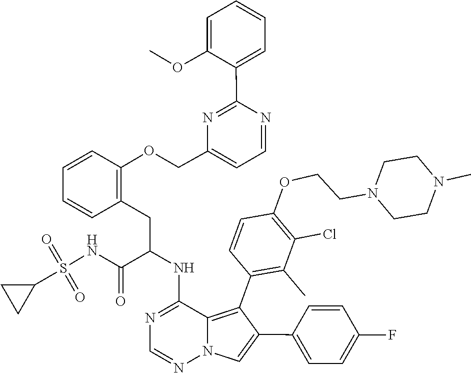 [SMILES: COc1ccccc1-c1nccc(COc2ccccc2CC(Nc2ncnn3cc(-c4ccc(F)cc4)c(-c4ccc(OCCN5CCN(C)CC5)c(Cl)c4C)c23)C(=O)NS(=O)(=O)C2CC2)n1] | 960.52 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)amino)-N- (cyclopropylsulfonyl)-3-(2-[2-(2- methoxyphenyl)pyrimidin-4- yl)methoxy)phenyl)propanamide |
| 27 | 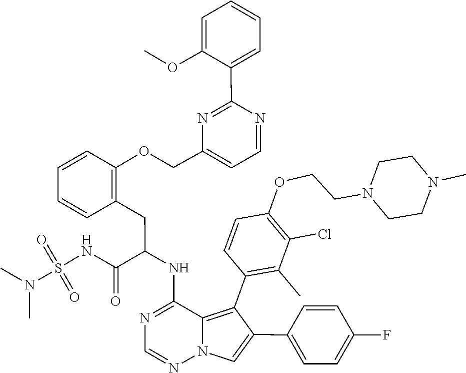 | 963.53 | 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)amino)-N-(N,N- dimethylsulfamoyl)-3-(2-((2-(2- methoxyphenyl)pyrimidin-4- yl)methoxy)phenyl)propanamide |
| 28 | 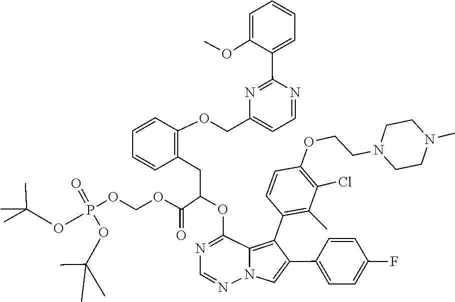 [SMILES: COc1ccccc1-c1nccc(COc2ccccc2CC(Oc2ncnn3cc(-c4ccc(F)cc4)c(-c4ccc(OCCN5CCN(C)CC5)c(Cl)c4C)c23)C(=O)OCOP(=O)(OC(C)(C)C)OC(C)(C)C)n1] | 1080.59 | ((di-tert-butoxyphosphoryl)oxy)methyl 2-((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-3-(2-((2-(2- methoxyphenyl)pyrimidin-4- yl)methoxy)phenyl)propanoate |
| 29 | 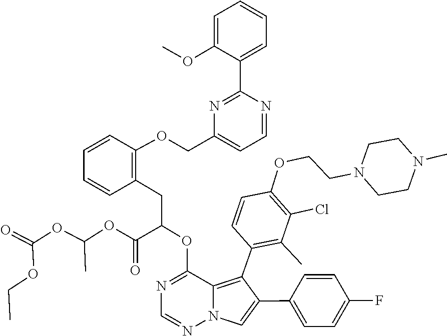 [SMILES: CCOC(=O)OC(C)OC(=O)C(Cc1ccccc1OCc1ccnc(-c2ccccc2OC)n1)Oc1ncnn2cc(-c3ccc(F)cc3)c(-c3ccc(OCCN4CCN(C)CC4)c(Cl)c3C)c12] | 974.48 | 1-((ethoxycarbonyl)oxy)ethyl 2-((5-[3- chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-3-(2-((2-(2- methoxyphenyl)pyrimidin-4- yl)methoxy)phenyl)propanoate |
| 30 | 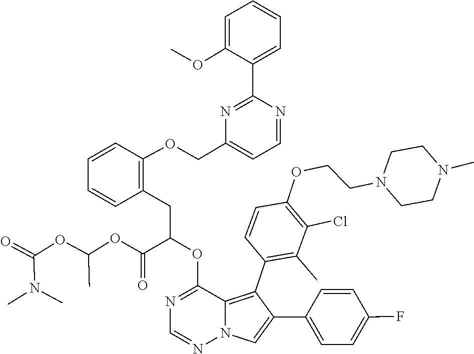 [SMILES: COc1ccccc1-c1nccc(COc2ccccc2CC(Oc2ncnn3cc(-c4ccc(F)cc4)c(-c4ccc(OCCN5CCN(C)CC5)c(Cl)c4C)c23)C(=O)OC(C)OC(=O)N(C)C)n1] | 973.50 | 1-((dimethylcarbamoyl)oxy)ethyl 2- ((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)oxy)-3-(2-((2-(2- methoxyphenyl)pyrimidin-4- yl)methoxy)phenyl)propanoate |
| 31 | 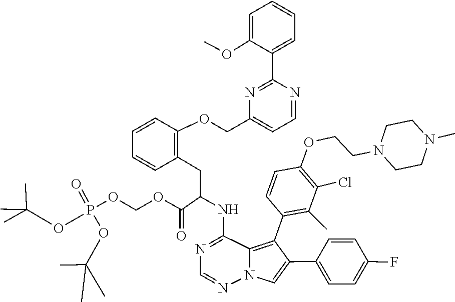 [SMILES: COc1ccccc1-c1nccc(COc2ccccc2CC(Nc2ncnn3cc(-c4ccc(F)cc4)c(-c4ccc(OCCN5CCN(C)CC5)c(Cl)c4C)c23)C(=O)OCOP(=O)(OC(C)(C)C)OC(C)(C)C)n1] | 1079.61 | ((di-tert-butoxyphosphoryl)oxy)methyl 2-((5-(3-chloro-2-methyl-4-(2-(4- methylpiperazin-l-yl)ethoxy)phenyl)- 6-(4-fluorophenyl)pyrrolo[2,1- f][1,2,4]triazin-4-yl)amino)-3-(2-((2-(2- methoxyphenyl)pyrimidin-4- yl)methoxy)phenyl)propanoate |
| 32 | 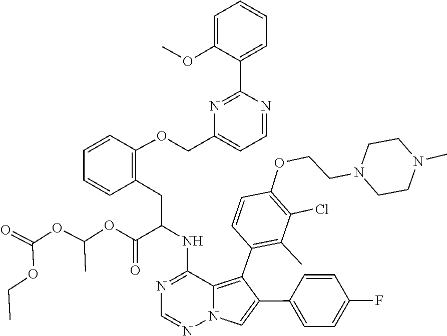 [SMILES: CCOC(=O)OC(C)OC(=O)C(Cc1ccccc1OCc1ccnc(-c2ccccc2OC)n1)Nc1ncnn2cc(-c3ccc(F)cc3)c(-c3ccc(OCCN4CCN(C)CC4)c(Cl)c3C)c12] | 973.50 | 1-((ethoxycarbonyl)oxy)ethyl 2-((5-(3- chloro-2-methyl-4-(2-(4- methylpiperazin-1-yl)ethoxy)phenyl)- 6-(4-fluorophenyl)pyrrolo[2,1- f][1,2,4]triazin-4-yl)amino)-3-(2-((2-(2- methoxyphenyl)pyrimidin-4- yl)methoxy)phenyl)propanoate |
| 33 | 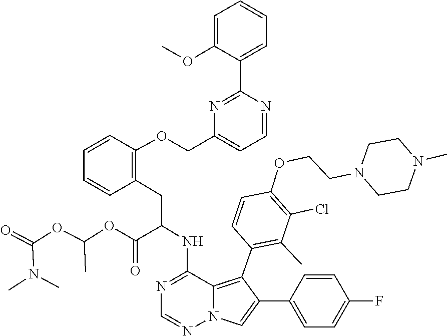 [SMILES: COc1ccccc1-c1nccc(COc2ccccc2CC(Nc2ncnn3cc(-c4ccc(F)cc4)c(-c4ccc(OCCN5CCN(C)CC5)c(Cl)c4C)c23)C(=O)OC(C)OC(=O)N(C)C)n1] | 972.52 | 1-((dimethylcarbamoyl)oxy)ethyl 2- ((5-(3-chloro-2-methyl-4-(2-(4- methylpiperazin-1-yl)ethoxy)phenyl)- 6-(4-fluorophenyl)pyrrolo[2,1- f][1,2,4]triazin-4-yl)amino)-3-(2-((2-(2- methoxyphenyl)pyrimidin-4- yl)methoxy)phenyl)propanoate |

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., 1H or 13C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

A series of pyrrolo-triazine derivatives of formula 10 can be prepared by the methods outlined in Scheme I. Suzuki coupling of compound 1 with suitable boronic acid Ar¹(OH)₂ or boronic ester Ar¹(OR)₂ (R=alkyl) results in the formation of the compound 2, which can be transformed to the dibromide compound 3 by treatment with NBS. Protecting SEM group in 4 can be achieved by reaction of 3 with SEMCl under basic condition (e.g., NaH in DMF, KHMDS in THF, NaHMDS in THF etc. . . . ). Treatment of the dibromo compound 4 with strong base such as butyl lithium, or t-butyl lithium can afford the mono-bromide 5 which can be converted to the corresponding 6 in acid condition such as TFA in DCM. Reaction of 6 with O-(diphenylphosphinyl)hydroxylamine 7 under basic conditions can yield compound 8 which can be converted to the corresponding pyrrolo-triazinone 9 by reaction with formamide. Treatment of 9 with POCl₃ or PCl₅ can form the pyrrolo-triazine 10.

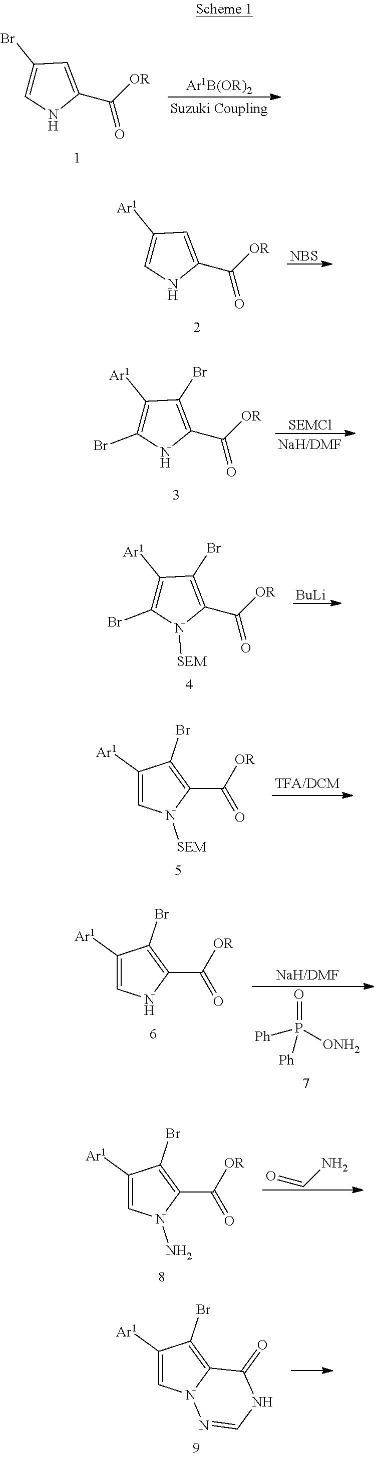

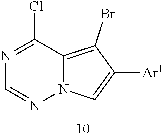

A series of pyrrole-2-carboxylate of formula 6 can be prepared by the methods outlined in Scheme 2. Suzuki coupling of compound 11 with suitable boronic acid Ar¹(OH)₂ or boronic ester Ar¹(OR)₂ (R=alkyl) results in the formation of the compound 12, which can be transformed to the pyrrole-2-carboxylate 13 by treatment with LDA at low temperature and then follows with chloroformate. Removal of the phenylsulfonyl group in 13 can give the pyrrole-2-carboxylate 6.

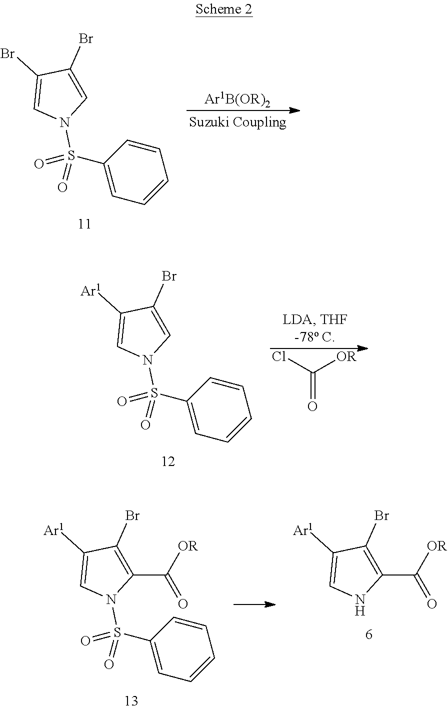

Alternatively, a series of pyrrolo-triazine of formula 10 can be prepared by the methods outlined in Scheme 3. Suzuki coupling of compound 14 with suitable boronic acid Ar¹(OH)₂ or boronic ester Ar¹(OR)₂ (R=alkyl) results in the formation of the compound 15, which can be transformed to the pyrrole-2-carboxylate 16 by treatment with POCl₃ or PCl₅. Treatment of 16 with NBS can produce the corresponding dibromo pyrrolo-triazine 17. Removal of one bromine atom from compound 17 can give the pyrrolo-triazine 10.

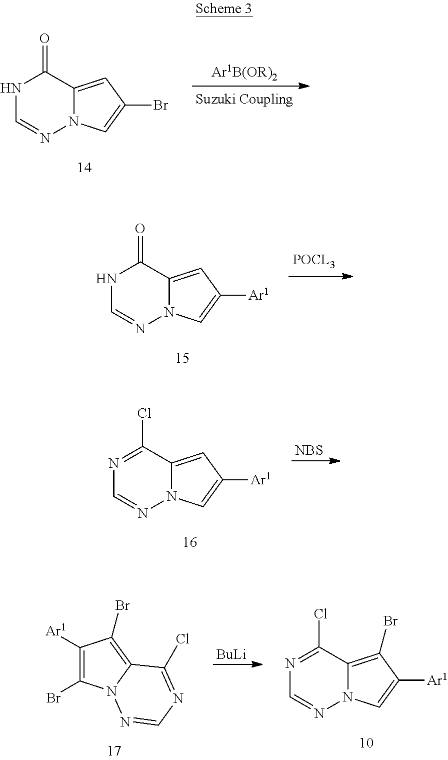

A series of pyrrolo-pyridazine compounds of formula 6 can be prepared by the methods outlined in Scheme 4. Bromo-pyrrolyl ketone 19 can be obtained by treatment of the pyrrolyl ketone 18 with NBS. The ketone 19 can be conveniently transformed to the enaminone 20 by heating with N,N-dimethylformamide dimethyl acetal or N,N-dimethylformamide diethyl acetal. Reaction of the enaminone 20 with O-(4-nitrobenzoyl)hydroxylamine 21 can give the corresponding pyrrolo-pyridazin-4-ol 22 in the presence of base such as potassium tert-butoxide, or sodium tert-butoxide. Reaction of 22 with SEMCl give the corresponding OH protecting compound 23 which can be transformed to compound 24 by Suzuki coupling with suitable boronic acid Ar¹(OH)₂ or boronic ester Ar¹(OR)₂ (R=alkyl). Treatment 24 with NBS can produce the dibromo compound 25 which can be transformed to the mono-bromo pyrrolo-pyridazine 23 by treatment with butyl lithium or other suitable strong basic reagent at low temperature. The pyrrolo-pyridazine 28 can be obtained by treatment of the OH derivative 27 which can be obtained by removal of the SEM group of 26 under acid conditions.

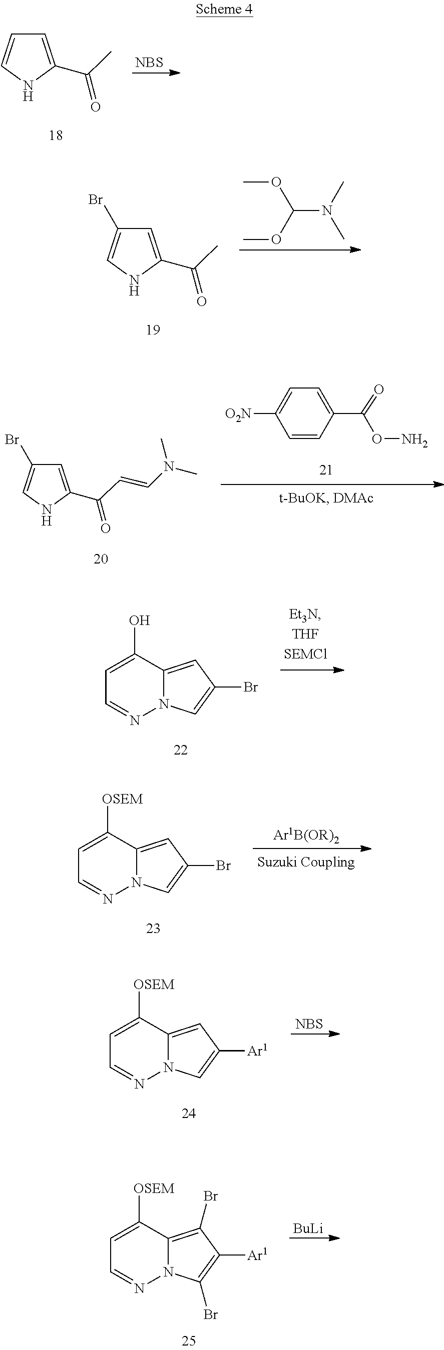

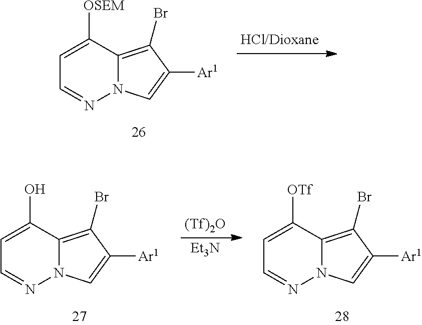

Alternatively, pyrrolo-pyridazin-4-ol 22 can be prepared by the methods outlined in Scheme 5. Reaction of 1-amino-pyrrole-2-carboxylate 29 with 3,3-diethoxypropanenitrile 30 can produce the corresponding pyrrolo-pyridazine 31. The cyano group in 31 can be then transformed to the amide 32. Removal of the amide group in 32 can afford the pyrrolo-pyridazin-4-ol 22.

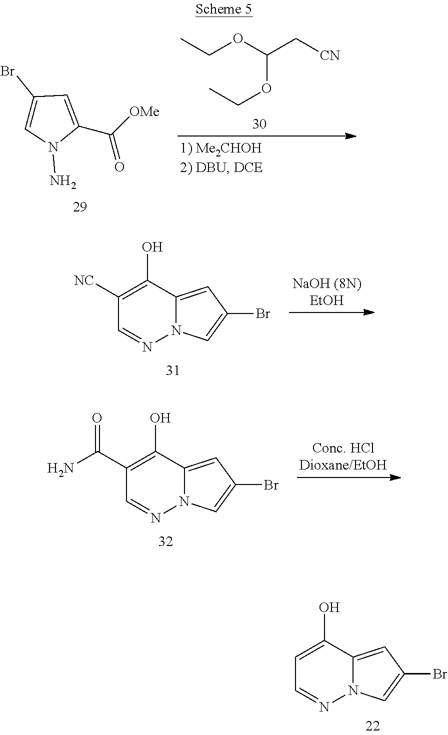

In a similar manner, pyrrolo-pyridazin-4-ol 22 can be prepared by the methods outlined in Scheme 6. Reaction of 1-amino-pyrrole-2-carboxylate 29 with enol ether 33 can produce the corresponding pyrrolo-pyridazine 34. Hydrolysis of the ethyl ester group in 34 to 35 and follow the de-carboxylate under acid condition can afford the pyrrolo-pyridazin-4-ol 22.

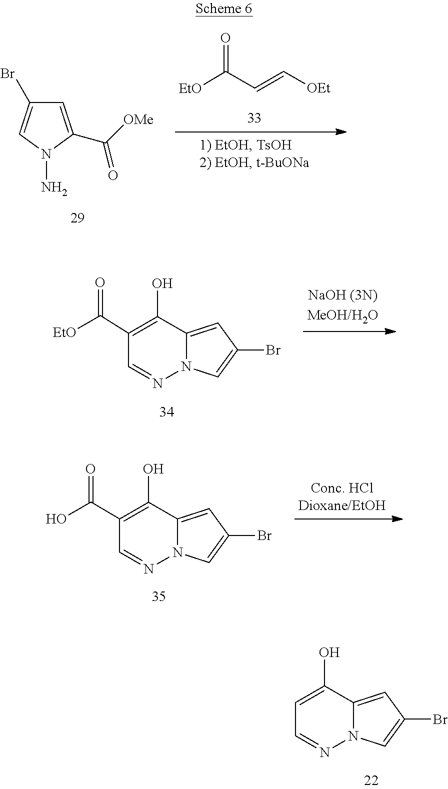 [SMILES: COC(=O)c1cc(Br)cn1N ; CCO/C=C/C(=O)OCC ; CCOC(=O)c1cnn2cc(Br)cc2c1O ; O=C(O)c1cnn2cc(Br)cc2c1O ; Oc1ccnn2cc(Br)cc12]

A series of acid derivatives of formula 41 can be prepared by the methods outlined in Scheme 7. Replacement of compound 10 or 28 with alpha-OH carboxylate ester 36 can yield the corresponding product 37. Suzuki coupling of compound 37 with suitable boronic acid Ar²(OH)₂ or boronic ester Ar²(OR)₂ (R=alkyl) can produce compound 38. The protecting group (PG=THP, MOM, PMB etc.) in compound 38 can be removed by treatment with suitable acid such as, but not limited to, HCl in methanol or dioxane, TFA in DCM to provide the corresponding phenol derivative 39. Mitsunobo reaction of 39 with the suitable alcohol R¹CH₂OH, or nucleophile of the 39 with R¹CH₂X (X is the leaving group such as Cl, Br, I, OMs or OTs, etc.) can afford the corresponding carboxylate ester 40 which can be conveniently hydrolyzed to the corresponding acid 41.

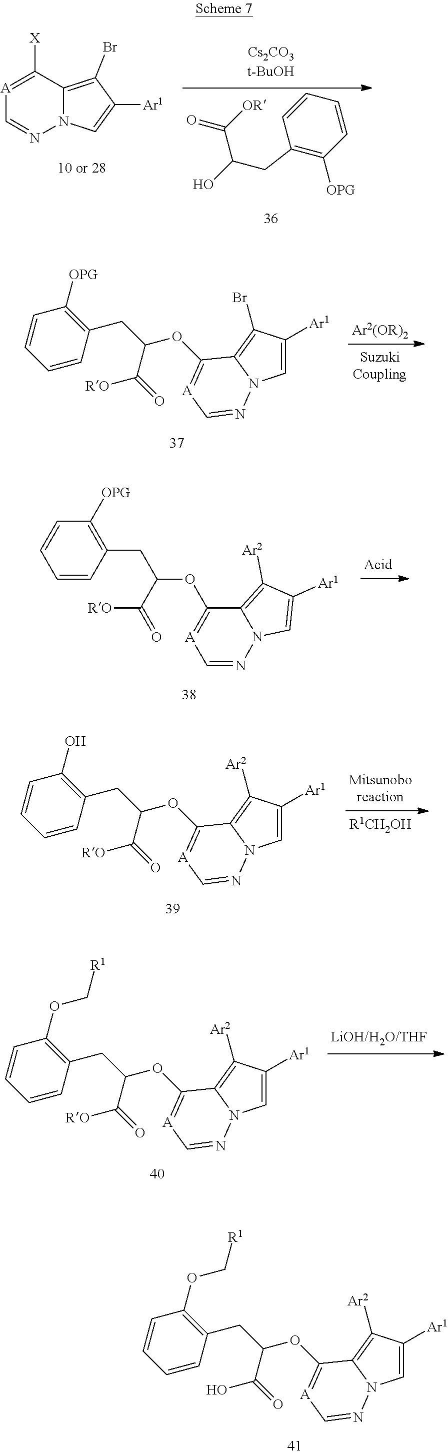

Similarly, a series of amino acid derivatives of formula 45 can be prepared by the methods outlined in Scheme 8. Replacement of compound 10 or 28 with alpha-amino carboxylate ester 42 can yield the corresponding product 43. Suzuki coupling of compound 43 can afford compound 44 which can be transformed into the corresponding acid 45.

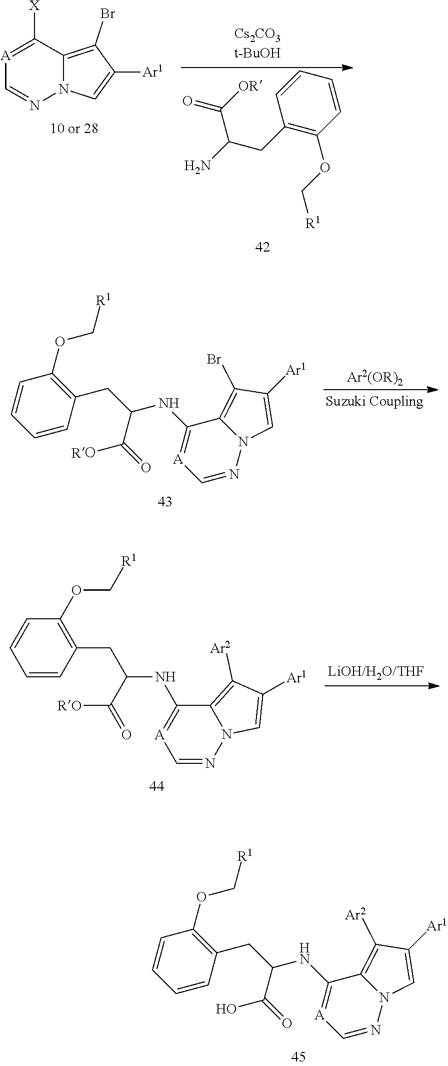

A series of acyl-sulfonamide derivatives of formula 47 can be prepared by the methods outlined in Scheme 9. The acid derivatives of formula 41 (B═O) or 45 (B═NH) can be conveniently transformed into the desired acyl-sulfonamide derivatives of formula 47 by reaction with the suitable sulfonamide derivative 46 (W═C or N) in the presence of amide coupling reagent such as, but not limited to, BOP, HATU, HBTU, EDCI, CDI, etc. . . . with the using base such as triethylamine, Hunig's base, DMAP, pyridine and so on.

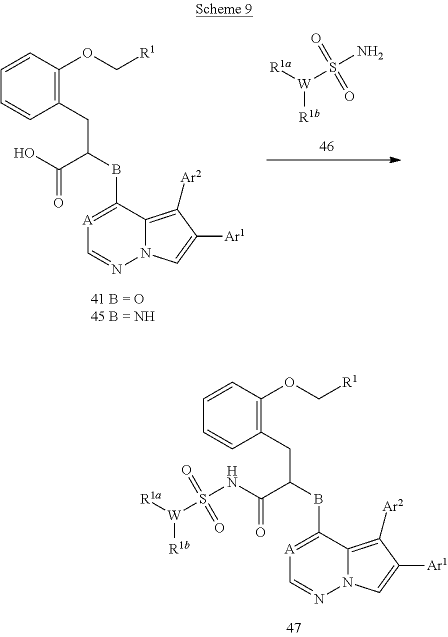

A series of carboxylate esters of formula 49 can be prepared by the methods outlined in Scheme 10. The carboxylate esters of formula 49 can be conveniently prepared by reaction of the acid derivatives of formula 41 (B═O) or 45 (B═NH) with chloride 48 (Z═C(O)OR^{a1}, C(O)NR^{c1}R^{d1}, or P(O)OR^{e}OR^{f}) under base conditions.

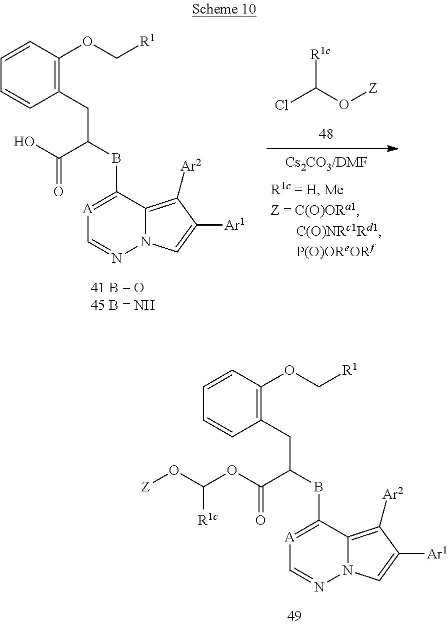

### Example 1

### (2R)-2-[(5Sₐ)-5-[3-Chloro-2-methyl-4-[2-(4-methyl piperazin-1-yl)ethoxy]phenyl]-6-(4-fluoro-phenyl)pyrrolo[2,1-f][1,2,4]triazin-4-yl]oxy-3-[2-(cyclopropylmethoxy)phenyl]propanoic acid

### Example 2

### (2S)-2-[(5R^{a})-5-[3-Chloro-2-methyl-4-[2-(4-methylpiperazin-1-yl)ethoxyl]phenyl]-6-(4-fluoro-phenyl)pyrrolo[2,1-f][1,2,4]triazin-4-yl]oxy-3-[2-(cyclopropylmethoxy)phenyl]propanoic acid

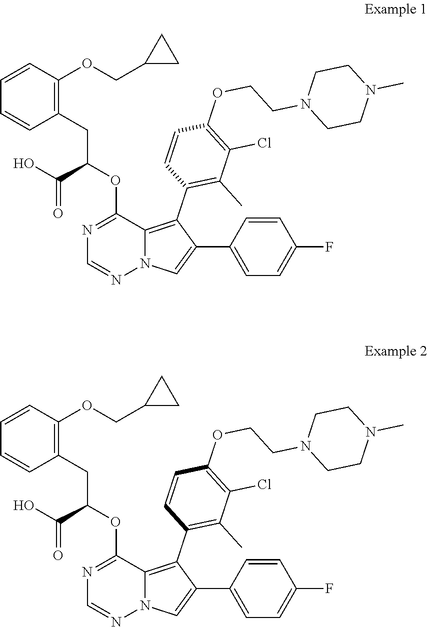

### Step 1: (2R)-3-[2-(cyclopropylmethoxy)phenyl]-2-hydroxy propanoate and methyl (2S)-3-[2-(cyclopropylmethoxy)phenyl]-2-hydroxy propanoate

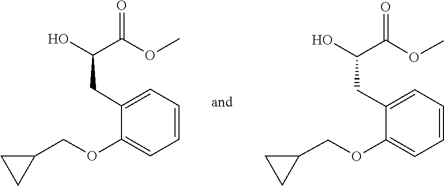 [SMILES: COC(=O)[C@H](O)Cc1ccccc1OCC1CC1 ; COC(=O)[C@@H](O)Cc1ccccc1OCC1CC1]

To a solution of bromomethylcyclopropane (0.65 g, 4.82 mmol) in DMF (40 mL) was added methyl (2R)-2-hydroxy-3-(2-hydroxyphenyl)propanoate (Intermediate 3, 860.0 mg, 4.38 mmol) and potassium carbonate (956.48 mg, 6.93 mmol). The reaction mixture was stirred at 70° C. for 18 h. After cooling, the mixture was filtered through a pad of celite. The filtrate was concentrated under reduced pressure and the residue was purified by flash chromatography on a silica gel column (PE/EA=2/1) to give a mixture of methyl (2R)-3-[2-(cyclopropylmethoxy)-phenyl]-2-hydroxy-propanoate methyl (2S)-3-[2-(cyclopropylmethoxy)phenyl]-2-hydroxy-propanoate (460 mg, 41.9% yield) as a light oil in a ratio of 66:33 (Retention time: t=2.002 and 2.212 min., respectively) as showed by Chiral analytic HPLC: Chiral Column: AD3, 5 um, 3 mm×150 mm; Mobile phase A: Supercritical CO₂, Mobile phase B: i-PrOH, A:B=90:10; Run time: 3 min.; Detector Wavelength: 254 nm. ¹H NMR: (400 MHz, CDCl₃) δ 7.20 (dt, J=1.6, 8.0 Hz, 1H), 7.15 (dd, J=6.6, 1.6 Hz, 1H), 6.90 (dt, J=1.0, 7.4 Hz, 1H), 6.83 (d, J=8.2 Hz, 1H), 4.54-4.52 (m, 1H), 3.89-3.81 (m, 2H), 3.73 (s, 3H), 3.26-3.21 (m, 2H), 3.01 (dd, J=13.8, 8.0 Hz, 1H), 1.33-1.24 (m, 1H), 0.67-0.62 (m, 2H), 0.41-0.34 (m, 2H).

### Step 2: methyl (2R)-2-((5-bromo-6-(4 fluorophenyl)pyrrolo[2,1f][1,2,4]triazin-4-yl)oxy)-3-(2-(cyclopropylmethoxy)phenyl)propanoate and methyl (2S)-2-((5-bromo-6-(4 fluorophenyl)-pyrrolo[2,1-f][1,2,4]triazin-4-yl)oxy)-3-(2-(cyclopropylmethoxy)phenyl)propanoate

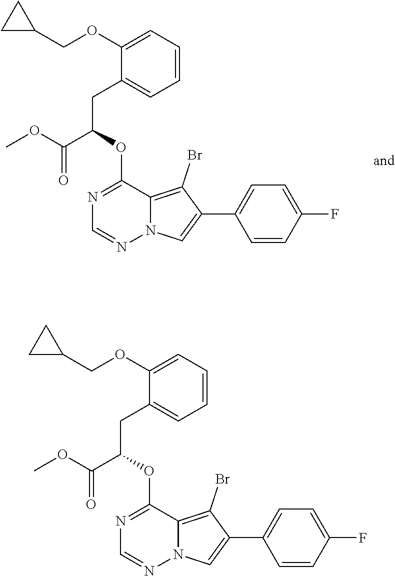 [SMILES: COC(=O)[C@@H](Cc1ccccc1OCC1CC1)Oc1ncnn2cc(-c3ccc(F)cc3)c(Br)c12 ; COC(=O)[C@H](Cc1ccccc1OCC1CC1)Oc1ncnn2cc(-c3ccc(F)cc3)c(Br)c12]

To a solution of methyl (2R)-3-[2-(cyclopropylmethoxy)phenyl]-2-hydroxy-propanoate and methyl (2S)-3-[2-(cyclopropylmethoxy)phenyl]-2-hydroxy-propanoate (430.0 mg, 1.72 mmol) in DMF (20 mL) was added 5-bromo-4-chloro-6-(4-fluorophenyl)pyrrolo[2,1-f][1,2,4]triazine (Intermediate 1, 617.12 mg, 1.89 mmol) and potassium carbonate (711.2 mg, 5.15 mmol), the reaction mixture was stirred at 70° C. for 18 h. After cooling, the mixture was filtered through a pad of celite. The filtrate was concentrated under reduced pressure and the residue was purified by flash chromatography on a silica gel column (PE/EA=1/1) to give methyl (2R)-2-[5-bromo-6-(4-fluorophenyl)pyrrolo[2,1-f][1,2,4]triazin-4-yl]oxy-3-[2-(cyclopropylmethoxy)phenyl]propanoate and methyl (2S)-2-[5-bromo-6-(4-fluorophenyl)pyrrolo[2,1-f][1,2,4]triazin-4-yl]oxy-3-[2-(cyclopropylmethoxy)phenyl]propanoate (650 mg, 70.% yield) as a solid. ¹H NMR (400 MHz, MeOD-d₄) δ 7.95 (s, 1H), 7.94 (s, 1H), 7.67-7.64 (m, 2H), 7.34 (dd, 0.1=7.4, 1.6 Hz, 1H), 7.21-7.16 (m, 3H), 6.91-6.83 (m, 2H), 5.85 (dd, J=9.2, 4.6, Hz, 1H), 3.91-3.87 (m, 3H), 3.73 (s, 3H), 3.54-3.50 (m, 1H), 1.31-1.26 (m, 1H), 0.63-0.59 (m, 2H), 0.40-0.37 (m, 2H).

### Step 3: methyl (2R)-2-[(5Sa)-5-[3-chloro-2-methyl-4-[2-(4-methylpiperazin-1-yl)ethoxy]phenyl]-6-(4 fluorophenyl)pyrrolo[2,1-f][1,2,4]triazin-4-yl]oxy-3-[2-(cyclopropylmethoxy)phenyl]propanoate and its enantiomer methyl (2S)-2-[(5Ra)-5-[3-chloro-2 methyl-4-[2-(4-methylpiperazin-1-yl)ethoxy]phenyl]-6-(4 fluorophenyl)pyrrolo[2,1f][1,2,4]triazin-4-yl]oxy-3-[2-(cyclopropylmethoxy)phenyl]propanoate

### and methyl (2S)-2-[(5Sa)-5-[3-chloro-2-methyl-4-[2-(4-methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoate and its enantiomer (2R)-2-[(5Ra)-5-[3-chloro-2-methyl-4-[2-(4-methylpiperazin-1-yl)ethoxy]phenyl]-6-(4 fluorophenyl)pyrrolo[2,1f][1,2,4]triazin-4-yl]oxy-3-[2-(cyclopropylmethoxy)phenyl]propanoate

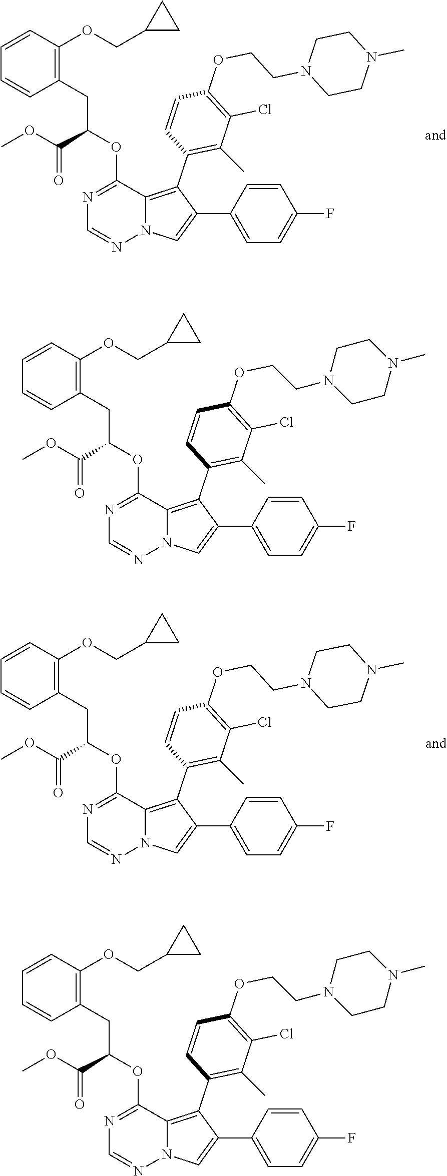

To a solution of 1-[2-[2-chloro-3-methyl-4-(4,4,5,5-tetramethyl-1,3,2-dioxaborolan-2-yl)phenoxy]ethyl]-4-methyl-piperazine (Intermediate 2, 712.22 mg, 1.8 mmol) and a mixture of methyl (2R)-2-[5-bromo-6-(4-fluorophenyl)pyrrolo[2,1-f][1,2,4]triazin-4-yl]oxy-3-[2-(cyclopropyl-methoxy)phenyl]propanoate and methyl (2S)-2-[5-bromo-6-(4-fluorophenyl)pyrrolo[2,1-f][1,2,4]triazin-4-yl]oxy-3-[2-(cyclopropylmethoxy)phenyl]propanoate (650.0 mg, 1.2 mmol) in THE (4 mL) and water (1 mL) was added bis(di-tert-butyl(4-dimethylaminophenyl)phosphine)-dichloropalladium(II) (42.6 mg, 0.06 mmol) and K₃PO₄ (766 mg, 3.6 mmol) at r.t. in a sealable tube, the reaction mixture was de-gassed and recharged with N₂ for three cycles, and stirred at 70° C. under microwave irradiations for 3 h. The mixture was concentrated under reduced pressure. The residue was purified by flash chromatography on a silica gel column (DCM:McOH=100:6) to afford 420 mg of the product which was further purified by Prep-HPLC on C18 column (5 uM, 50×150 mm) with H₂O (0.1% TFA)/CH₃CN to give two groups of diastereomers P1 (the earlier eluted product) and P2 (the latter eluted product).

P1 was assigned as a mixture of methyl (2R)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate and its enantiomer methyl (2S)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (170 mg, 19.4% yield) in a ratio of 71:29 (Retention time: Rₜ=4.10 and 4.37 min., respectively) as showed by Chiral analytic HPLC: Chiral Column: OZ-H, 5 um, 4.6 mm×150 mm; Mobile phase: Hep:ETOH (0.1% DEA)=60:40; Run time: 10 min.; Detector Wavelength: 254 nm. LCMS calculated for C₄₀H₄₄ClFN₅O₅, [M+H]⁺: m/z=728.29; found: 728.10.

P2 was assigned as a mixture of methyl (2S)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate and its enantiomer methyl (2R)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (190 mg, 21.7% yield) in a ratio of 28:72 (Retention time: Rₜ=3.48 and 4.84 min., respectively) as showed by Chiral analytic HPLC: Chiral Column: IC-H, 5 um, 4.6 mm×150 mm; Mobile phase: Hep:EtOH (0.1% DEA)=60:40; Run time: 8 min.; Detector Wavelength: 254 nm. LCMS calculated for C₄₀H₄₄ClFN₅O₅, [M+H]⁺: m/z=728.29; found: 728.13.

### Step 4: methyl (2R)-2-[(5Sa)-5-[3-chloro-2-methyl-4-[2-(4-methylpiperazin-1-yl)ethoxy]phenyl]-6-(4 fluorophenyl)pyrrolo[2,1-f][1,2,4]triazin-4-yl]oxy-3-[2-(cyclopropylmethoxy)phenyl]propanoate

### and methyl (2S)-2-[(5Ra)-5-[3-chloro-2-methyl-4-[2-(4 methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoate

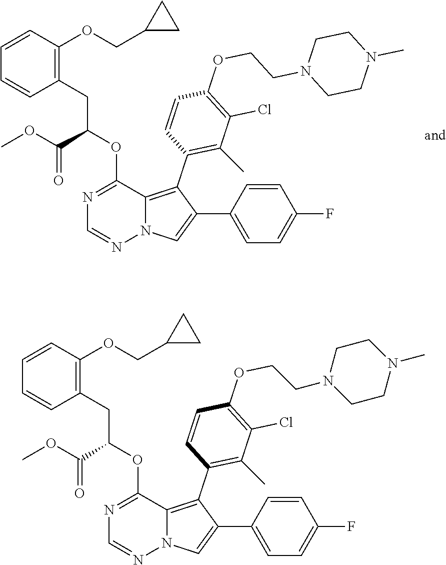

A mixture of methyl (2R)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropyl-methoxy)phenyl]propanoate and its enantiomer methyl (2S)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (P1 from Step 3, 170.0 mg, 0.23 mmol) was separated by Chiral-HPLC to give P1-1 (the earlier eluted product, 60 mg, Retention time=4.021 min. in chiral analytic HPLC) and P1-2 (the latter eluted product, 20 mg, Retention time=5.305 min. in chiral analytic HPLC). Chiral HPLC separation conditions: Instrument: Waters-SFC80; Column: OZ-H (30*250 mm, 5 um); Mobile phase A: Supercritical CO₂, Mobile phase B: EtOH (0.1% NH₃.H₂O), A:B=60/40; Flow rate: 65 mL/min; Circle Time: 15 min; Sample preparation: methanol; Injection Volume: 2.0 mL; Detector Wavelength: 220 nm; Column temperature: 25° C.; Back pressure: 100 bar. The separated products were determined by chiral HPLC. Chiral HPLC conditions: Chiral Column: OZ-H, 5 um, 4.6 mm×150 mm; Mobile phase: Hep:EtOH (0.1% DEA)=60:40; Flow rate: 0.5 mL/min and Run time: 10 min.; Detector Wavelength: 254 nm.

P1-1 was assigned to methyl (2R)-2-[(5Sa)-5-[3-chloro-2-methyl-4-[2-(4-methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoate. ¹H NMR: (400 MHz, Methanol-d₄) δ 8.04 (s, 1H), 7.92 (s, 1H), 7.23-7.18 (m, 2H), 7.10-7.06 (m, 1H), 7.00-6.93 (m, 2H), 6.88 (dd, J=15.4, 8.5 Hz, 2H), 6.80 (d, J=7.8 Hz, 1H), 6.61 (dt, J=0.9, 7.4 Hz, 1H), 6.19 (dd, J=7.5, 1.5 Hz, 1H), 5.54 (dd, J=9.6, 3.8 Hz, 1H), 4.29-4.21 (m, 2H), 3.86-3.79 (m, 2H), 3.70 (s, 3H), 3.25 (dd, J=13.6, 3.6 Hz, 1H), 2.91 (t, J=5.4 Hz, 2H), 2.85-2.39 (m, 9H), 2.33 (s, 3H), 2.27 (s, 3H), 1.27-1.20 (m, 1H), 0.62-0.58 (m, 2H), 0.40-0.35 (m, 2H).

And P1-2 was assigned to methyl (2S)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate. ¹H NMR: (400 MHz, Methanol-d₄) δ 8.05 (s, 1H), 7.93 (s, 1H), 7.23-7.19 (m, 2H), 7.10-7.06 (m, 1H), 7.00-6.94 (m, 2H), 6.88 (dd, J=15.6, 8.5 Hz, 2H), 6.80 (d, J=8.0 Hz, 1H), 6.61 (dt, J=0.8, 7.4 Hz, 1H), 6.19 (dd, J=7.4, 1.6 Hz, 1H), 5.54 (dd, J=9.6, 3.8 Hz, 1H), 4.28-4.21 (m, 2H), 3.86-3.79 (m, 2H), 3.70 (s, 3H), 3.25 (dd, J=13.8, 3.8 Hz, 1H), 2.91 (t, J=5.4 Hz, 2H), 2.84-2.39 (m, 9H), 2.34 (s, 3H), 2.29 (s, 3H), 1.26-1.22 (m, 1H), 0.61-0.58 (m, 2H), 0.38-0.35 (m, 2H).

### Step 5: (2R)-2-[(5Sa)-5-[3-chloro-2-methyl-4-[2-(4-methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoic acid

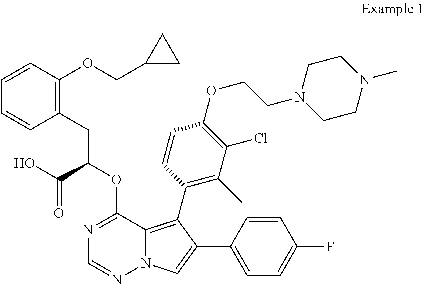

To a solution of methyl (2R)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropyl-methoxy)phenyl]propanoate (P1-1 Step 4, 60 mg, 0.08 mmol) in THE (6 mL) was added LiOH (2.0 mL, 4.0 mmol, 2 M in H₂O). The reaction mixture was stirred at r.t. for 6 h. The mixture was adjusted with 1 M of HCl to pH 6, diluted with water (15 mL) and extracted with EA (3×20 mL). The organics were dried over sodium sulfate, filtered and evaporated under reduced pressure to afford (2R)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoic acid (50 mg, 85% yield) with ee %=100% as showed by chiral HPLC with AD3 column. LCMS calculated for C₃₉H₄₂ClFN₅O₅ [M+H]⁺: m/z=714.28; found: 713.93. ¹H NMR: (400 MHz, Methanol-d₄) δ 8.03 (s, 1H), 7.94 (s, 1H), 7.22-7.18 (m, 2H), 7.09-7.05 (m, 1H), 6.98-6.80 (m, 5H), 6.59 (t, J=7.5 Hz, 1H), 6.12 (d, J=7.2 Hz, 1H), 5.55-5.52 (m, 1H), 4.42-4.35 (m, 2H), 3.85-3.80 (m, 2H), 3.57-3.32 (m, 9H), 3.28-3.23 (m, 2H), 2.92-2.91 (m, 3H), 2.55-2.49 (m, 1H), 2.37 (s, 3H), 1.30-1.27 (m, 1H), 0.62-0.57 (m, 2H), 0.39-0.37 (m, 2H).

### Step 6: (2S)-2-[(5Ra)-5-[3-chloro-2 methyl-4-[2-(4-methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoic acid

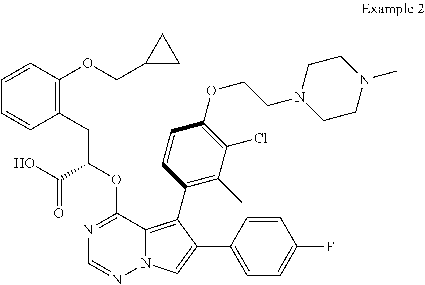

This compound was prepared using procedures analogous to those described for Step 5 using methyl (2S)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (P1-2 Step 4, 20 mg) to afford (2S)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropyl-methoxy)phenyl]propanoic acid (5 mg). LCMS calculated for C₃₉H₄₂ClFN₅O₅ [M+H]⁺: m/z=714.28; found: 714.04. ¹H NMR: (400 MHz, Methanol-d₄) δ 8.03 (s, 1H), 7.94 (s, 1H), 7.21-7.18 (m, 2H), 7.09-7.05 (m, 1H), 6.98-6.90 (m, 3H), 6.87-6.79 (m, 2H), 6.58 (t, J=7.2 Hz, 1H), 6.10 (dd, J=7.6, 1.4 Hz, 1H), 5.52 (dd, J=10.2, 3.2 Hz, 1H), 4.31-4.23 (m, 2H), 3.85-3.82 (m, 2H), 3.39-3.34 (m, 2H), 3.28-3.15 (m, 7H), 3.05 (t, J=5.0 Hz, 2H), 2.83 (s, 3H), 2.52 (dd, J=13.8, 10.4 Hz, 1H), 2.37 (s, 3H), 1.31-1.22 (m, 1H), 0.62-0.58 (m, 2H), 0.39-0.35 (m, 2H). ee %=100% as showed by chiral HPLC with AD3 column.

### Example 3

### (2S)-2-[(5Sa)-5-[3-Chloro-2-methyl-4-[2-(4-methyl piperazin-1-yl)ethoxy]phenyl]-6-(4-fluoro-phenyl)pyrrolo[2,1-f][1,2,4]triazin-4-yl]oxy-3-[2-(cyclopropylmethoxy)phenyl]propanoic acid

### And Example 4

### (2R)-2-[(5Ra)-5-[3-Chloro-2-methyl-4-[2-(4-methylpiperazin-1-yl)ethoxy]phenyl]-6-(4-fluoro-phenyl)pyrrolo[2,1-f][1,2,4]triazin-4-yl]oxy-3-[2-(cyclopropylmethoxy)phenyl]propanoic acid

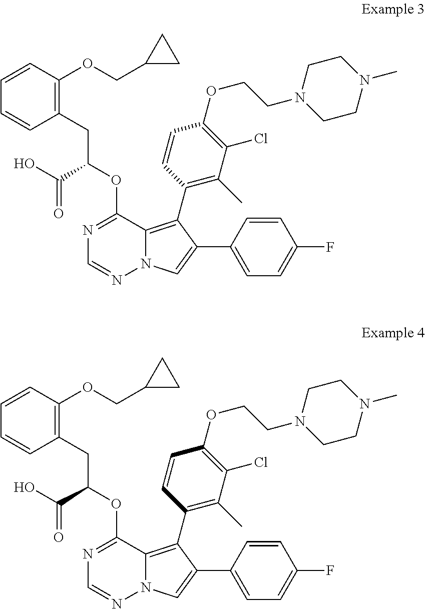

### Step 1: methyl (2S)-2-[(5Sa)-5-[3-chloro-2 methyl-4-[2-(4 methylpiperazin-1-yl)ethoxy]phenyl]-6-(4 fluorophenyl)pyrrolo[2,1f][1,2,4]triazin-4-yl]oxy-3-[2-(cyclopropylmethoxy)phenyl]propanoate

### and methyl (2R)-2-[(5Ra)-5-[3-chloro-2-methyl-4-[2-(4-methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoate

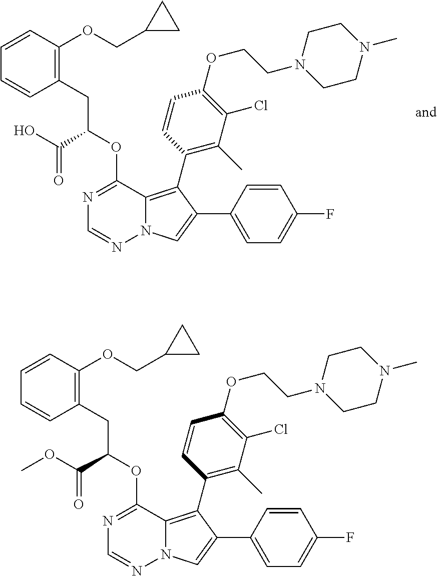

A mixture of methyl (2S)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropyl-methoxy)phenyl]propanoate and its enantiomer methyl (2R)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (P2 from Example 1 Step 3, 190 mg, 0.26 mmol) was separated by Chiral HPLC separation to afford P2-1 (the earlier peak, 40 mg, Retention time=3.349 min. in analytical chiral-HPLC) and P2-2 (the latter peak, 100 mg, Retention time=4.660 min in analytical chiral-HPLC). The Chiral HPLC separation was performed on Waters-SFC80 instrument under the separation conditions: Column: IC-H (30*250 mm, 5 um); Mobile phase A: Supercritical CO₂. Mobile phase B: EtOH (0.1% NH₃.H₂O), A:B=60/40; Flow rate: 50 mL/min; Sample preparation: methanol; Injection Volume: 2.0 mL; Detector Wavelength: 220 nm; Column temperature: 25° C.; Back pressure: 100 bar. The separated products were determined by chiral analytic HPLC. Chiral analytic HPLC conditions: Chiral Column: IC-H, 5 um, 4.6 mm×150 mm; Mobile phase: HEP:EtOH (0.1% DEA)=60:40; Flow rate: 0.5 mL/min and Detector Wavelength: 254 nm.

P2-1 was assigned to methyl (2S)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropyl-methoxy)phenyl]propanoate. ¹H NMR: (400 MHz, MeOD-d₄) δ 8.03 (s, 1H), 7.92 (s, 1H), 7.42 (d, J=8.5 Hz, 1H), 7.23-7.20 (m, 2H), 7.12 (d, J=8.5 Hz, 1H), 7.06 (dt, J=1.5, 7.8 Hz, 1H), 6.99-6.95 (m, 2H), 6.79 (d, J=7.8 Hz, 1H), 6.61 (dt, J=0.9, 7.4 Hz, 1H), 6.06 (dd, J=7.4, 1.6 Hz, 1H), 5.55 (dd, J=7.4, 3.2 Hz, 1H), 4.32 (t, J=5.2 Hz, 2H), 3.88-3.79 (m, 2H), 3.75 (s, 3H), 3.28-3.26 (m, 1H), 2.93 (t, J=5.2 Hz, 2H), 2.88-2.35 (m, 9H), 2.27 (s, 3H), 1.79 (s, 3H), 1.28-1.24 (m, 1H), 0.63-0.58 (m, 2H), 0.39-0.36 (m, 2H).

And P2-2 was assigned to methyl (2R)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate. ¹H NMR: (400 MHz, MeOD-d₄) δ 8.04 (d, J=2.5 Hz, 1H), 7.93 (d, J=1.8 Hz, 1H), 7.44 (dd, J=8.6, 0.8 Hz, 1H), 7.23-7.20 (m, 2H), 7.13 (d, J=8.6 Hz, 1H), 7.07 (dt, J=1.6, 7.8 Hz, 1H), 6.99-6.95 (m, 2H), 6.80 (d, J=8.3 Hz, 1H), 6.62 (dt, J=0.8, 7.4 Hz, 1H), 6.06 (dd, J=7.4, 1.6 Hz, 1H), 5.55 (dd, J=10.4, 3.4 Hz, 1H), 4.33 (t, J=5.0 Hz, 2H), 3.88-3.78 (m, 2H), 3.74 (s, 3H), 3.46-3.36 (m, 2H), 3.28-3.24 (m, 1H), 3.23-2.94 (m, 6 H), 2.82 (s, 3H), 2.76-2.56 (m, 2H), 2.51 (dd, J=13.5, 10.2 Hz, 1H), 1.80 (s, 3H), 1.26-1.22 (m, 1H), 0.63-0.60 (m, 2H), 0.40-0.36 (m, 2H).

### Step 2: (2S)-2-[(5Sa)-5-[3-chloro-2-methyl-4-[2-(4 methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoic acid

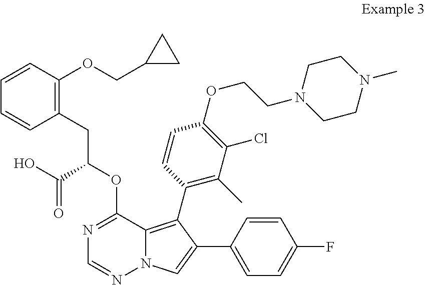

This compound was prepared using procedures analogous to those described for Example 1 Step 5 using methyl (2S)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (P2-1 Step 1, 40 mg) to afford (2S)-2-[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoic acid (37 mg, 94.3% yield). LCMS calculated for C₃₉H₄₂ClFN₅O₅ [M+H]⁺: m/z=714.28; found: 714.07. ¹H NMR: (400 MHz, Methanol-d₄) δ 8.02 (s, 1H), 7.94 (s, 1H), 7.48 (d, J=8.4 Hz, 1H), 7.23-7.19 (m, 2H), 7.15 (d, J=8.5 Hz, 1H), 7.08-7.04 (m, 1H), 7.00-6.94 (m, 2H), 6.80 (d, J=8.2 Hz, 1H), 6.62-6.58 (m, 1H), 6.00 (dd, J=7.4, 1.6 Hz, 1H), 5.55 (dd, J=10.8, 2.8 Hz, 1H), 4.35 (t, J=4.8 Hz, 2H), 3.88-3.80 (m, 2H), 3.38 (dd, J=13.4, 2.7 Hz, 1H), 3.30-3.16 (m, 5H), 3.14-3.00 (m, 5H), 2.83 (s, 3H), 2.46 (dd, J=13.5, 10.8 Hz, 1H), 1.78 (s, 3H), 1.29-1.24 (m, 1H), 0.63-0.58 (m, 2H), 0.40-0.37 (m, 2H). ee %=100% as showed by chiral HPLC with AD3 column.

### Step 3: (2R)-2-[(5Ra)-5-[3-chloro-2-methyl-4-[2-(4-methylpiperazin-1-yl)ethoxy]phenyl]-6-(4-fluorophenyl)pyrrolo[2,1f][1,2,4]triazin-4-yl]oxy-3-[2-(cyclopropylmethoxy)phenyl]propanoic acid

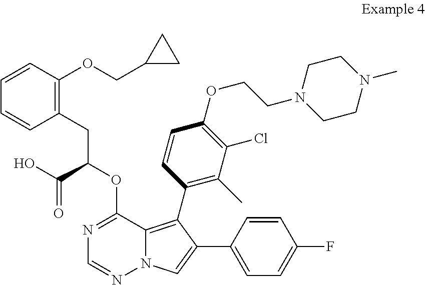

This compound was prepared using procedures analogous to those described for Example 1 Step 5 using methyl (2R)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoate (P2-2 Step 1, 100 mg) to afford (2R)-2-[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]oxy-3-[2-(cyclopropylmethoxy)phenyl]propanoic acid (78.1 mg, 78.0% yield). LCMS calculated for C₃₉H₄₂ClFN₅O₅ [M+H]⁺: m/z=714.28; found: 713.90. ¹H NMR: (400 MHz, Methanol-d₄) δ 7.90 (s, 1H), 7.89 (s, 1H), 7.65 (d, J=8.5 Hz, 1H), 7.20-7.16 (m, 2H), 7.10 (d, J=8.6 Hz, 1H), 6.99-6.92 (m, 3H), 6.74 (d, J=7.7 Hz, 1H), 6.52 (dt, J=0.9, 7.4 Hz, 1H), 5.97 (dd, J=7.5, 1.5 Hz, 1H), 5.40 (dd, J=11.2, 2.6 Hz, 1H), 4.30 (t, J=5.2 Hz, 2H), 3.85-3.77 (m, 2H), 3.30 (dd, J=13.9, 2.5 Hz, 1H), 2.92-2.90 (m, 2H), 2.79-2.44 (m, 9H), 2.26-2.25 (m, 3H), 1.71 (s, 3H), 0.95-0.90 (m, 1H), 0.59-0.55 (m, 2H), 0.36-0.33 (m, 2H). ee %=100% as showed by chiral HPLC with AD3 column.

### Example 5

### (2R)-2-{[(5Sa)-5-[3-Chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoic acid

### And Example 6

### (2S)-2-{[(5Ra)-5-[3-Chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoic acid

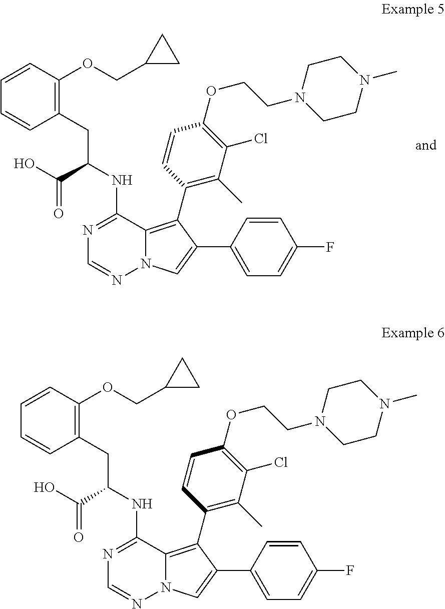

### Step 1: methyl (2R)-2-((tert-butoxycarbonyl)amino)-3-(2-(cyclopropylmethoxy)phenyl)propanoate and methyl (2S)-2-((tert-butoxycarbonyl)amino)-3-(2-(cyclopropylmethoxy)phenyl)propanoate

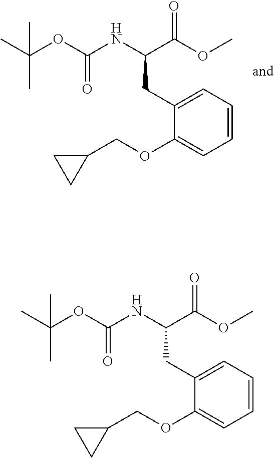 [SMILES: COC(=O)[C@@H](Cc1ccccc1OCC1CC1)NC(=O)OC(C)(C)C ; COC(=O)[C@H](Cc1ccccc1OCC1CC1)NC(=O)OC(C)(C)C]

To a solution of bromomethylcyclopropane (482.71 mg, 3.58 mmol) in DMF (25 mL) was added methyl (2R)-2-(tert-butoxycarbonylamino)-3-(2-hydroxyphenyl)propanoate and its enantiomer methyl (2S)-2-(tert-butoxycarbonylamino)-3-(2-hydroxyphenyl)propanoate (Intermediate 4, 960 mg, 3.3 mmol) and potassium carbonate (956 mg, 6.9 mmol). The reaction mixture was stirred at room temperature for 18 h. and filtered. The filtrate was concentrated under reduced pressure and the residue was purified by flash chromatography on a silica gel column (PE/EA=2/1) to give a mixture of methyl (2R)-2-(tert-butoxycarbonylamino)-3-[2-(cyclopropylmethoxy)phenyl]propanoate and its enantiomer methyl (2S)-2-(tert-butoxycarbonylamino)-3-[2-(cyclopropylmethoxy)phenyl]propanoate (500 mg, 44.0% yield) as a light oil. ¹H NMR: (400 MHz, DMSO-d₆) δ 7.19-7.11 (m, 3H) 6.92 (d, J=8.0 Hz, 1H), 6.83 (t, J=7.4 Hz, 1H), 4.28-4.25 (m, 1H), 3.87-3.85 (m, 2H), 3.57 (s, 3H), 3.07 (dd, J=13.0, 5.0 Hz, 1H), 2.75 (dd, J=13.5, 9.6 Hz, 1H), 1.31 (s, 9H), 1.23-1.21 (m, 1H), 0.58-0.54 (m, 2H), 0.39-0.35 (m, 2H).

### Step 2: methyl (R)-2-amino-3-(2-(cyclopropylmethoxy)phenyl)propanoate hydrochloride and methyl (S)-2-amino-3-(2-(cyclopropylmethoxy)phenyl)propanoate hydrochloride

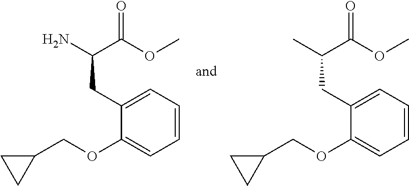 [SMILES: COC(=O)[C@H](N)Cc1ccccc1OCC1CC1 ; COC(=O)[C@@H](C)Cc1ccccc1OCC1CC1]

To a solution of methyl (2R)-2-(tert-butoxycarbonylamino)-3-[2-(cyclopropylmethoxy)-phenyl]propanoate and its enantiomer methyl (2S)-2-(tert-butoxycarbonylamino)-3-[2-(cyclopropylmethoxy)phenyl]propanoate (500 mg, 1.43 mmol) in 1,4-dioxane (5 mL) was added HCl/Dioxane (4.0 N, 20 mL, 80 mmol) at r.t. The reaction mixture was stirred at r.t. for 3 h, the solvent was removed to afford the crude product as a pale white solid. The crude solid was washed with McOBu-t/DCM (40 mL/3 mL) to afford methyl (R)-2-amino-3-(2-(cyclopropylmethoxy)phenyl)propanoate hydrochloride and methyl (S)-2-amino-3-(2-(cyclopropylmethoxy)phenyl)propanoate hydrochloride (400 mg, 97.8% yield) as a white solid. ¹H NMR: (400 MHz, DMSO-d₆) δ 8.56 (br s, 3H), 7.27-7.22 (m, 1H), 7.15 (dd, J=7.2, 1.6 Hz, 1H), 6.97 (dt, J=0.8, 7.4 Hz, 1H), 4.16 (t, J=7.3 Hz, 1H), 3.85 (d, J=6.7 Hz, 2H), 3.73-3.64 (m, 1H), 3.61 (s, 3H), 3.59-3.49 (m, 1H), 3.17-3.05 (m, 2H), 1.27-1.20 (m, 1H), 0.59-0.53 (m, 2H), 0.36-0.33 (m, 2H).

### Step 3: methyl (2R)-2-[[5-bromo-6-(4 fluorophenyl)pyrrolo[2,1f][1,2,4]triazin-4-yl]amino]-3-[2-(cyclopropylmethoxy)phenyl]propanoate and methyl (2S)-2-[[5-bromo-6-(4 fluorophenyl)-pyrrolo[2,1-f][1,2,4]triazin-4-yl]amino]-3-[2-(cyclopropylmethoxy)phenyl]propanoate

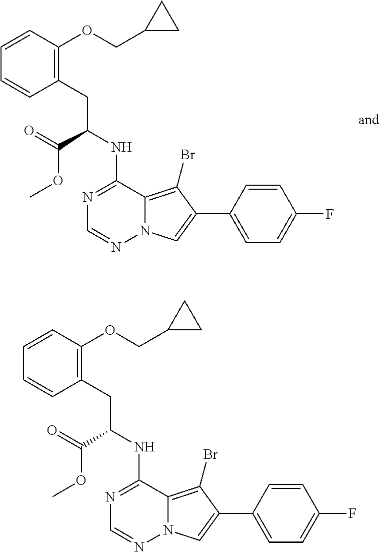 [SMILES: COC(=O)[C@@H](Cc1ccccc1OCC1CC1)Nc1ncnn2cc(-c3ccc(F)cc3)c(Br)c12 ; COC(=O)[C@H](Cc1ccccc1OCC1CC1)Nc1ncnn2cc(-c3ccc(F)cc3)c(Br)c12]

To a solution of methyl (2R)-2-amino-3-[2-(cyclopropylmethoxy)phenyl]propanoate hydrochloride and methyl (2S)-2-amino-3-[2-(cyclopropylmethoxy)phenyl]propanoate hydrochloride (400.0 mg, 1.4 mmol), and 5-bromo-4-chloro-6-(4-fluorophenyl)pyrrolo[2,1-f][1,2,4]triazine (Intermediate 1, 548 mg, 1.68 mmol) in toluene (20 mL) was added triethylamine (1.3 mL, 9.3 mmol). The mixture was stirred at 110° C. for 18 h. The solvent was removed by reduced pressure and the residue was purified by flash chromatography on a silica gel column (PE/EA=3/1) to give the product methyl (2R)-2-[[5-bromo-6-(4-fluorophenyl)-pyrrolo[2,1-f][1,2,4]triazin-4-yl]amino]-3-[2-(cyclopropylmethoxy)phenyl]propanoate and its enantiomer methyl (2S)-2-[[5-bromo-6-(4-fluorophenyl)pyrrolo[2,1-f][1,2,4]triazin-4-yl]amino]-3-[2-(cyclopropylmethoxy)phenyl]propanoate (530 mg, 70.2% yield) as a solid. ¹H NMR (400 MHz, DMSO-d₆) δ 8.09 (s, 1H), 7.96 (s, 1H), 7.65-7.62 (m, 2H), 7.34-7.29 (m, 2H), 7.21-7.14 (m, 3H), 6.94 (d, J=7.7 Hz, 1H), 6.86 (dt, J=0.8, 7.4 Hz, 1H), 5.20-5.15 (m, 1H), 3.86-3.76 (m, 2H), 3.71 (s, 3H), 3.45-3.40 (m, 1H), 3.20-3.14 (m, 1H), 1.10-1.06 (m, 1H), 0.50-0.48 (m, 2H), 0.30-0.27 (m, 2H).

### Step 4: methyl (2S)-2-{[(5Ra)-5-[3-chloro-2-methyl-4-[2-(4-methylpiperazin-1-yl)ethoxy]phenyl]-6-(4 fluorophenyl)pyrrolo[2,1-f][1,2,4]triazin-4-yl]amino}-3-[2-(cyclopropylmethoxy)phenyl]-propanoate and its enantiomer methyl (2R)-2-{[(5Sa)-5-[3-chloro-2-methyl-4-[2-(4-methylpiperazin-1-yl)ethoxy]phenyl]-6-(4 fluorophenyl)pyrrolo[2,1f][1,2,4]triazin-4-yl]amino}-3-[2-(cyclopropylmethoxy)phenyl]propanoate

### and methyl (2R)-2-{[(5Ra)-5-[3-chloro-2-methyl-4-[2-(4 methylpiperazin-1-yl)ethoxy]phenyl]-6-(4-fluorophenyl)pyrrolo[2,1f][1,2,4]triazin-4-yl]amino}-3-[2-(cyclopropylmethoxy)phenyl]-propanoate and its enantiomer methyl (2S)-2-{[(5Sa)-5-[3-chloro-2-methyl-4-[2-(4-methylpiperazin-1-yl)ethoxy]phenyl]-6-(4 fluorophenyl)pyrrolo[2,1f][1,2,4]triazin-4-yl]amino}-3-[2-(cyclopropylmethoxy)phenyl]propanoate

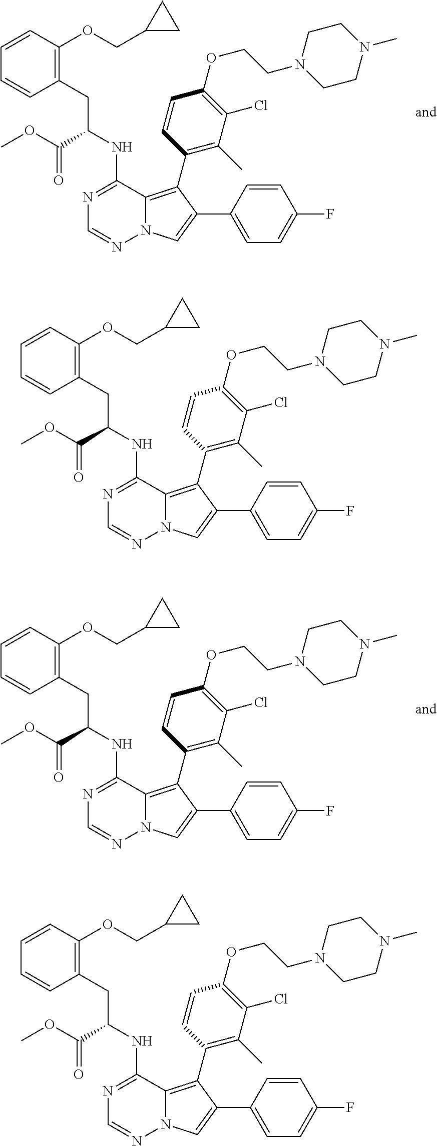

To a solution of 1-[2-[2-chloro-3-methyl-4-(4,4,5,5-tetramethyl-1,3,2-dioxaborolan-2-yl)phenoxy]ethyl]-4-methyl-piperazine (Intermediate 2, 582 mg, 1.47 mmol) and methyl (2R)-2-{[5-bromo-6-(4-fluorophenyl)pyrrolo[2,1-f][1,2,4]triazin-4-yl]amino}-3-[2-(cyclopropylmethoxy)-phenyl]propanoate and its enantiomer methyl (2S)-2-{[5-bromo-6-(4-fluorophenyl)pyrrolo[2,1-f][1,2,4]triazin-4-yl]amino}-3-[2-(cyclopropylmethoxy)phenyl]propanoate (530 mg, 0.98 mmol) in THE (4 mL) and water (1 mL) was added bis(di-tert-butyl(4-dimethylaminophenyl)phosphine)-dichloropalladium(II) (34.8 mg, 0.05 mmol)) and K₃PO₄ (626 mg, 2.95 mmol) at r.t. in a seal tube. The reaction mixture was de-gassed and recharged with N₂ for three times and stirred at 70° C. for 18 h. The mixture was concentrated under reduced pressure. The residue was purified by flash chromatography on a silica gel column (DCM:MeOH=10:1) to afford a mixture of the products which were further purified by Pre-HPLC on C18 column (5 uM, 50 mm×150 mm) with H₂O (0.1% TFA)/CH₃CN to give two groups of diastereomers P1 (the earlier eluted product, 120 mg) and P2 (the latter eluted product, 150 mg).

P1 was assigned as a mixture of methyl (2S)-2-{[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate and its enantiomer methyl (2R)-2-{[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate in a ratio of 44:56 (Retention time: t=8.04 and 9.14 min., respectively) as showed by Chiral analytic HPLC: Chiral Column: TRefoil™ AMY1, 2.5 μm, 3.0 mm×150 mm; Mobile phase A: Supercritical CO₂, Mobile phase B: i-PrOH, A/B=85:15; Run time: 15 min.; Detector Wavelength: 254 nm; Instrument: Waters Acuity UPC². ¹H NMR of the first peak: ¹H NMR: (400 MHz, MeOD-d₄) δ 7.92-7.89 (m, 2H), 7.26-7.16 (m, 3H), 6.97-6.91 (m, 4H), 6.83-6.79 (m, 1H), 6.74-6.72 (m, 1H), 6.64 (d, J=8.6 Hz, 1H), 5.12 (t, J=5.4 Hz, 1H), 4.33-4.24 (m, 1H), 4.20-4.12 (m, 1H), 3.73-3.66 (m, 2H), 3.66 (s, 3H), 3.57-3.52 (m, 1H), 3.49-3.33 (m, 5H), 3.26-3.05 (m, 5H), 2.91-2.89 (m, 3H), 2.84-2.79 (m, 1H), 2.02 (s, 3H), 0.94-0.86 (m, 1H), 0.51-0.40 (m, 2H), 0.24-0.18 (m, 1H), 0.09-0.03 (m, 1H).

And P2 was assigned as a mixture of methyl (2R)-2-{[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate and its enantiomer methyl (2S)-2-{[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate in a ratio of 56:44 (Retention time: t=10.18 and 11.34 min., respectively) as showed by Chiral analytic HPLC: Chiral Column: TRefoil™ AMY1, 2.5 μm, 3.0 mm×150 mm; Mobile phase A: Supercritical CO₂, Mobile phase B: i-PrOH, A/B=85:15; Run time: 15 min.; Detector Wavelength: 254 nm; Instrument: Waters Acuity UPC². ¹H NMR: (400 MHz, MeOD-d₄) δ 7.86-7.85 (m, 2H), 7.34 (d, J=8.4 Hz, 1H), 7.16-7.08 (m, 4H), 6.96-6.91 (m, 2H), 6.79 (d, J=8.1 Hz, 1H), 6.70 (t, J=7.4 Hz, 1H), 6.48 (d, J=7.2 Hz, 1H), 5.12 (dd, J=8.0, 5.2 Hz, 1H), 4.34 (dd, J=11.0, 6.0 Hz, 2H), 3.73-3.68 (m, 5H), 3.40-3.33 (m, 6H), 3.21-3.08 (m, 5H), 2.88 (d, J=2.5 Hz, 3H), 2.66-2.61 (m, 1H), 1.79 (s, 3H), 1.16-1.09 (m, 1H), 0.59-0.52 (m, 2H), 0.32-0.24 (m, 2H).

### Step 5: (2R)-2-{[(5Sa)-5-[3-chloro-2-methyl-4-[2-(4-methylpiperazin-1-yl)ethoxy]phenyl]-6-(4-fluorophenyl)pyrrolo[2,1f][1,2,4]triazin-4-yl]amino}-3-[2-(cyclopropylmethoxy)phenyl]propanoic acid and (2S)-2-{([(5Ra)-5-[3-chloro-2 methyl-4-[2-(4-methylpiperazin-1-yl)ethoxy]phenyl]-6-(4-fluorophenyl)pyrrolo[2,1f][1,2,4]triazin-4-yl]amino}-3-[2-(cyclopropylmethoxy)phenyl]propanoic acid

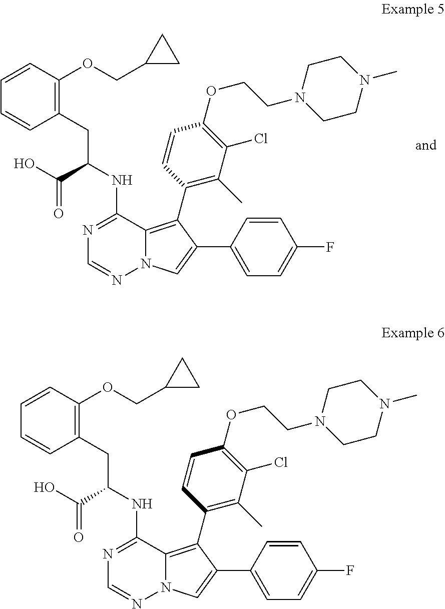

To a solution of methyl (2S)-2-{[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate and its enantiomer methyl (2R)-2-{[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate (P1 Step 4, 120.0 mg, 0.17 mmol) in THE (15 mL) and water (1 mL) was added LiOH (8.22 mL, 16.43 mmol) (2 M in H₂O). The reaction mixture was stirred at r.t. for 2 h. The mixture was adjusted with 1 M HCl to pH=6. To the mixture was added 15 mL water and extracted with EA (3×20 mL). The organic layers were dried over sodium sulfate, filtered and evaporated to give a crude product. The crude product was further purified by Pre-HPLC on C18 column to afford the mixture of the acids in a ratio of 58:42 on Chiral AD-H column. The acids were separated by Chiral HPLC to afford P1-1 (the earlier eluted product, 28.5 mg, Retention time t=4.672 min. in the chiral analytic AD-H column) and P1-2 (the latter eluted product, 26.5 mg, Retention time t=5.178 min. in the chiral analytic AD-H column) under Chiral HPLC separation conditions: Instrument: Waters-SFC80; Column: AD-H (30*250 mm, 5 um); Mobile phase A: Supercritical CO₂, Mobile phase B: EtOH (0.1% NH₃.H₂O), A:B=60/40; Flow rate: 50 mL/min; Circle Time: 15 min; Sample preparation: methanol; Injection Volume: 1.0 mL; Detector Wavelength: 220 nm; Column temperature: 38° C.; Back pressure: 100 bar; Retention time: 7.0 and 9.0 min., respectively. The separated products were determined by chiral HPLC. Chiral HPLC conditions: Chiral Column: AD-H, 5 um, 4.6 mm×150 mm; Mobile phase: Hep:EtOH (0.1% DEA)=60:40; Flow rate: 0.5 mL/min and Run time: 10 min.; Detector Wavelength: 254 nm.

P1-1 was assigned to Example 5: (2R)-2-{[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoic acid. LCMS calculated for C₃₉H₄₃ClFN₆O₄, [M+H]⁺: m/z=713.29; found: 713.5. ¹H NMR: (400 MHz, Methanol-d₄) δ 7.88 (s, 1H), 7.80 (s, 1H), 7.36-7.32 (m, 1H), 7.25-7.19 (m, 3H), 7.02-6.86 (m, 6H), 6.18 (d, J=8.5 Hz, 1H), 4.77 (dd, J=5.1, 2.6 Hz, 1H), 4.16 (t, J=10.4 Hz, 1H), 4.02-3.98 (m, 1H), 3.93 (dd, J=13.0, 5.2 Hz, 1H), 3.68 (dd, J=10.0, 7.0 Hz, 1H), 3.49 (dd, J=10.0, 6.9 Hz, 2H), 3.36-3.34 (m, 1H), 3.04 (dd, J=15.4, 2.4 Hz, 2H), 2.93 (dd, J=13.2, 2.6 Hz, 2H), 2.86-2.78 (m, 4H), 2.76 (s, 3H), 1.83 (s, 3H), 0.65-0.58 (m, 1H), 0.39-0.32 (m, 1H), 0.27-0.20 (m, 1H), 0.08-0.02 (m, 1H), −0.21-−0.27 (m, 1H).

And P1-2 was assigned to Example 6: (2S)-2-{[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoic acid. LCMS calculated for C₃₉H₄₃ClFN₆O₄, [M+H]⁺: m/z=713.29; found: 713.5. ¹H NMR: (400 MHz, Methanol-d₄) δ 7.88 (s, 1H), 7.81 (s, 1H), 7.34-7.30 (m, 1H), 7.24-7.20 (m, 2H), 7.16-7.12 (m, 1H), 7.00-6.85 (m, 6H), 6.24 (d, J=8.3 Hz, 1H), 4.16 (t, J=10.2 Hz, 1H), 4.06-4.01 (m, 1H), 3.88 (dd, J=13.2, 5.3 Hz, 1H), 3.68 (dd, J=10.0, 7.0 Hz, 1H), 3.51 (dd, J=10.0, 7.0 Hz, 2H), 3.29-3.26 (m, 1H), 3.06-3.02 (m, 2H), 2.91 (dd, J=13.3, 3.0 Hz, 2H), 2.88-2.81 (m, 4H), 2.78 (s, 3H), 1.86 (s, 3H), 0.68-0.61 (m, 1H), 0.41-0.34 (m, 1H), 0.30-0.23 (m, 3H), 0.11-0.04 (m, 1H), −0.15-−0.22 (m, 1H).

### Example 7

### (2R)-2-{[(5Ra)-5-[3-Chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]-propanoic acid

### and Example 8

### (2S)-2-{[(5Sa)-5-[3-Chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]-propanoic acid

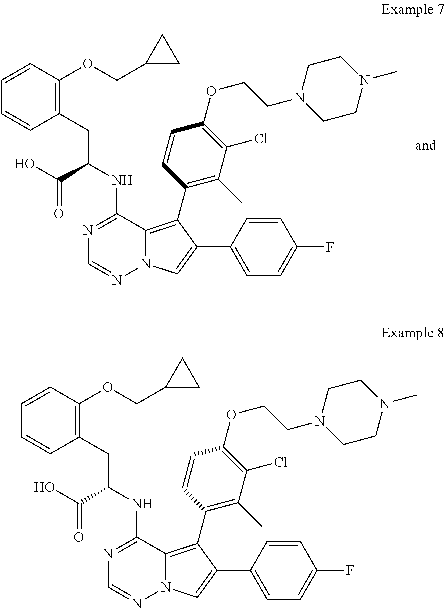

This compound was prepared using procedures analogous to those described for Example 5 Step 5 using methyl (2R)-2-{[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate and its enantiomer methyl (2S)-2-{[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoate (P2, Example 5 Step 4, 150 mg) The crude mixture of Example 7 and Example 8 was purified by Chiral-HPLC separation to afford P2-1 (the earlier eluted product, 34.5 mg, Retention time Rt=6.47 min. in the chiral analytic IC-H column) and P2-2 (the latter eluted product, 28.6 mg, Retention time Rt=7.76 min. in the chiral analytic IC-H column) under Chiral HPLC separation conditions: Instrument: Waters-SFC80; Column: IC-H (30*250 mm, 5 um); Mobile phase A: Supercritical CO₂. Mobile phase B: EtOH (0.1% NH₃.H₂O), A:B=60/40; Flow rate: 50 mL/min; Circle Time: 15 min; Sample preparation: methanol; Injection Volume: 1.0 mL; Detector Wavelength: 220 nm; Column temperature: 38° C.; Back pressure: 100 bar; Retention time: 6.61 and 7.99 min., respectively. The separated products were determined by chiral HPLC. Chiral HPLC conditions: Chiral Column: IC-H, 5 um, 4.6 mm×150 mm; Mobile phase: Hep:EtOH (0.1% DEA)=60:40; Flow rate: 0.5 mL/min and Run time: 10 min.; Detector Wavelength: 254 nm.

P2-1 was assigned to Example 7: (2R)-2-{[(5Ra)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoic acid as a white solid. ee %=100% as showed by chiral HPLC. LCMS calculated for C₃₉H₄₃ClFN₆O₄, [M+H]⁺: m/z=713.29; found: 713.27. ¹H NMR: (400 MHz, MeOD-d₄) δ 7.82 (s, 1H), 7.77 (s, 1H), 7.25 (d, J=8.5 Hz, 1H), 7.19-7.15 (m, 2H), 7.07-7.01 (m, 2H), 6.95-6.90 (m, 2H), 6.89-6.86 (m, 1H), 6.74-6.68 (m, 2H), 4.55-4.50 (m, 1H), 4.18-4.13 (m, 1H), 3.66-3.54 (m, 4H), 3.22-3.16 (m, 1H), 3.14-2.93 (m, 9H), 2.84 (dd, J=13.9, 5.4 Hz, 1H), 2.70 (s, 3H), 1.83 (s, 3H), 0.99-0.89 (m, 1H), 0.50-0.45 (m, 2H), 0.23-0.15 (m, 1H), 0.15-0.08 (m, 1H).

And P2-2 was assigned to Example 8: (2S)-2-{[(5Sa)-5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino}-3-[2-(cyclopropylmethoxy)phenyl]propanoic acid as a white solid. ee %=100% as showed by chiral HPLC. LCMS calculated for C₃₉H₄₃ClFN₆O₄, [M+H]⁺: m/z=713.29; found: 712.89. ¹H NMR: (400 MHz, MeOD-d₄) δ 7.82 (s, 1H), 7.77 (s, 1H), 7.25 (d, J=8.5 Hz, 1H), 7.19-7.15 (m, 2H), 7.07-7.01 (m, 2H), 6.95-6.90 (m, 2H), 6.89-6.86 (m, 1H), 6.74-6.68 (m, 2H), 4.55-4.50 (m, 1H), 4.18-4.13 (m, 1H), 3.66-3.54 (m, 4H), 3.22-3.16 (m, 1H), 3.13-2.94 (m, 9H), 2.84 (dd, J=13.8, 5.3 Hz, 1H), 2.70 (s, 3H), 1.83 (s, 3H), 0.99-0.90 (m, 1H), 0.52-0.43 (m, 2H), 0.23-0.15 (m, 1H), 0.15-0.08 (m, 1H).

### Example 9

### (2R)-2-{[(5Sa)-5-{3-Chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoic acid

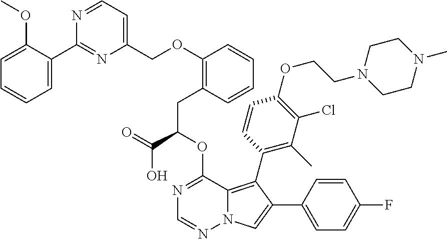

### Step 1: (2-(2 methoxyphenyl)pyrimidin-4-yl)methyl methanesulfonate

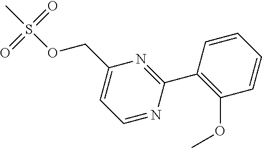 [SMILES: COc1ccccc1-c1nccc(COS(C)(=O)=O)n1]

To the solution of [2-(2-methoxyphenyl)pyrimidin-4-yl]methanol (280.0 mg, 1.29 mmol) (SpiroChem AG, Cat. #SPC-1109, CAS #1339058-28-4) in DCM (20 mL) and triethylamine (0.54 mL, 3.88 mmol), was added dropwise in DCM (5 mL) at 0° C. The reaction mixture was stirred at 0° C. for 3 h. The mixture was diluted with DCM (20 mL) and washed with water (20 mL) and brine. The organics was dried over anhydrous Na₂SO₄, filtered and concentrated under reduced pressure. The residue was purified by column chromatography on a silica gel column (PE/EA=1/1) to give [2-(2-methoxyphenyl)-pyrimidin-4-yl]methyl methanesulfonate (286 mg, 75% yield). ¹H NMR: (400 MHz, DMSO-d₆) δ 8.94 (d, J=5.2 Hz, 1H), 7.55-7.45 (m, 3H), 7.16 (d, J=7.6 Hz, 1H), 7.08-7.04 (m, 1H), 5.39 (s, 2H), 3.76 (s, 3H), 3.36 (s, 3H).

### Step 2: Methyl 2-hydroxy-3-(2-((2-(2-methoxyphenyl)pyrimidin-4-yl)methoxy)phenyl)propanoate

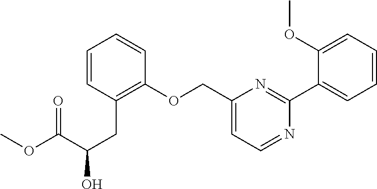 [SMILES: COC(=O)[C@H](O)Cc1ccccc1OCc1ccnc(-c2ccccc2OC)n1]

To a solution of methyl (2R)-2-hydroxy-3-(2-hydroxyphenyl)propanoate (Intermediate 3, 46.4 mg, 0.24 mmol) in DMF (50 mL) in a seal tube was added [2-(2-methoxyphenyl)pyrimidin-4-yl]methyl methanesulfonate (58.0 mg, 0.20 mmol) and potassium carbonate (54.4 mg, 0.39 mmol), the reaction mixture was stirred at 25° C. for 3 h, the solvent was removed under reduced pressure and the residue was purified by pre-TLC on silica gel (PE/EA=1/2) to give the product methyl (2R)-2-hydroxy-3-[2-[[2-(2-methoxyphenyl)pyrimidin-4-yl]methoxy]phenyl]propanoate (25 mg, 32.2% yield) as a light oil with ee 88.4% as showed by Chiral analytic HPLC in AD3 column. ¹H NMR: (400 MHz, DMSO-d₆) δ 8.92 (d, J=5.2 Hz, 1H), 8.75-7.45 (m, 3H), 7.23-7.15 (m, 3H), 7.08-7.03 (m, 2H), 6.93-6.89 (m, 1H), 5.59-5.52 (m, 1H), 5.25 (s, 2H), 4.39-4.32 (m, 1H), 3.77 (s, 3H), 3.61 (s, 3H), 3.19-3.15 (m, 1H), 2.88-2.83 (m, 1H).

### Step 3: methyl (R)-2-{[5-bromo-6-(4 fluorophenyl)pyrrolo[2,1-f][1,2,4]triazin-4-yl]oxy}-3-{2-[2-(2-methoxyphenyl)pyrimidin-4-yl]methoxyphenyl}propanoate

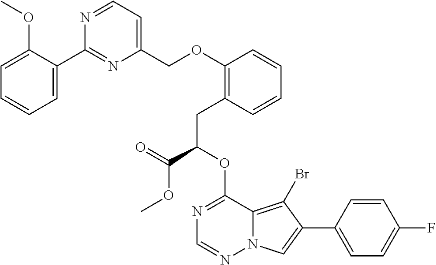 [SMILES: COC(=O)[C@@H](Cc1ccccc1OCc1ccnc(-c2ccccc2OC)n1)Oc1ncnn2cc(-c3ccc(F)cc3)c(Br)c12]

To a solution of 5-bromo-4-chloro-6-(4-fluorophenyl)pyrrolo[2,1-f][1,2,4]triazine (Intermediate 1, 483 mg, 1.48 mmol) in DMF (10 mL) in a seal tube was added methyl (R)-2-hydroxy-3-(2-{[2-(2-methoxyphenyl)pyrimidin-4-yl]methoxy}phenyl)propanoate (530 mg, 1.34 mmol) and potassium carbonate (556 mg, 4.03 mmol). The reaction mixture was stirred at 70° C. for 18 h. The solvent was removed under reduced pressure and the residue was purified by pre-TLC on silica gel (PE/EA=1/2) to give the product methyl (R)-2-{[5-bromo-6-(4-fluorophenyl)pyrrolo[2,1-f][1,2,4]triazin-4-yl]oxy}-3-{2-[2-(2-methoxyphenyl)pyrimidin-4-yl]methoxyphenyl}propanoate (650 mg, 70.7% yield) as a solid with ee 88.4% as showed by Chiral analytic HPLC in AD3 column. ¹H NMR (400 MHz, DMSO-d₆) δ 8.92 (d, J=5.2 Hz, 1H), 8.31 (s, 1H), 8.17 (s, 1H), 7.71-7.14 (m, 13H), 5.78-5.74 (m, 1H), 5.33-5.24 (m, 2H), 3.76 (s, 3H), 3.72 (s, 3H), 3.64-3.59 (m, 1H), 3.37-3.34 (m, 1H).

### Step 4: methyl (2R)-2-{[(5Sa)-5-{3-chloro-2-methyl-4-[2-(4-methylpiperazin-1-yl)ethoxy]phenyl}-6-(4 fluorophenyl)pyrrolo[2,1f][1,2,4]triazin-4-yl]oxy}-3-(2-{[2-(2-methoxyphenyl)pyrimidin-4-yl]methoxy}phenyl)propanoate and methyl (2R)-2-{([(5Ra)-5-{3-chloro-2-methyl-4-[2-(4-methylpiperazin-1-yl)ethoxy]phenyl}-6-(4 fluorophenyl)pyrrolo[2,1-f][1,2,4]triazin-4-yl]oxy}-3-(2-{[2-(2 methoxyphenyl)pyrimidin-4-yl]methoxy}phenyl)propanoate

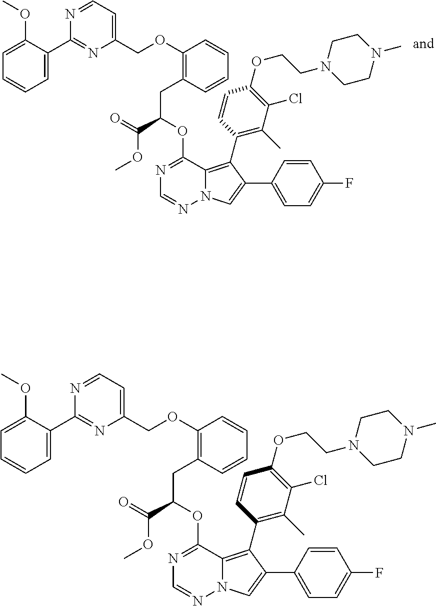

To a solution of 1-[2-[2-chloro-3-methyl-4-(4,4,5,5-tetramethyl-1,3,2-dioxaborolan-2-yl)phenoxy]ethyl]-4-methyl-piperazine (Intermediate 2, 199 mg, 0.50 mmol) and methyl (R)-2-{[5-bromo-6-(4-fluorophenyl)pyrrolo[2,1-f][1,2,4]triazin-4-yl]oxy}-3-{2-[2-(2-methoxyphenyl)-pyrimidin-4-yl]methoxyphenyl}propanoate (230 mg, 0.34 mmol) in THE (4 mL) and water (1 mL) was added bis(di-tert-butyl(4-dimethylaminophenyl)phosphine)dichloropalladium(II) (11.9 mg, 0.02 mmol)) and K₃PO₄ (95 mg, 0.45 mmol) at r.t. in a seal tube. The reaction mixture was de gassed and recharged with N₂ for three cycles and stirred at 65° C. for 2 h. under microwave irradiation. The mixture was concentrated under reduced pressure. The residue was purified by Pre-HPLC on C18 column to afford P1 (the earlier eluted product, 33 mg, 11.3% yield, Retention time t=2.11 min.) and P2 (the latter eluted product, 95 mg, 32.4% yield, Retention time t=2.14 min.).

P1 was assigned to methyl (2R)-2-{[(5Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoate. ¹H NMR (400 MHz, DMSO-d₆) δ 8.97 (d, J=5.1 Hz, 1H), 8.40 (s, 1H), 8.08 (s, 1H), 7.63 (d, J=5.1 Hz, 1H), 7.50 (dd, J=7.5, 1.7 Hz, 1H), 7.47-7.42 (m, 1H), 7.26-7.23 (m, 2H), 7.18-7.01 (m, 7H), 6.89 (d, J=8.5 Hz, 1H), 6.66 (t, J=7.4 Hz, 1H), 6.01 (dd, J=7.3, 1.6 Hz, 1H), 5.45 (dd, J=10.3, 2.9 Hz, 1H), 5.24 (dd, J=24.3, 15.2 Hz, 2H), 4.31-4.19 (m, 4H), 3.75 (s, 3H), 3.73 (s, 3H), 3.44-3.37 (m, 4H), 3.07-2.94 (m, 4H), 2.76 (s, 3H), 2.67-2.66 (m, 1H), 2.48-2.46 (m, 1H), 2.36 (s, 3H). P2 was assigned to methyl (2R)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoate. ¹H NMR: (400 MHz, DMSO-d₆) δ 8.91 (d, J=5.2 Hz, 1H), 8.14 (s, 1H), 7.93 (s, 1H), 755-7.44 (m, 3H), 7.30-6.99 (m, 11H), 6.81-6.77 (m, 1H), 6.51-6.48 (m, 1H), 5.37 (d, J=7.6 Hz, 1H), 5.17 (s, 2H), 5.05-5.00 (m, 1H), 4.26-4.22 (m, 2H), 3.76 (s, 3H), 3.65 (s, 3H), 3.41-3.37 (m, 1H), 2.79-2.67 (m, 4H), 2.54-2.50 (m, 2H), 2.33-2.31 (m, 4H), 2.12 (s, 3H), 1.74 (s, 3H).

### Step 5: (2R)-2-{[(5Sa)-5-{3-chloro-2 methyl-4-[2-(4 methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoic acid

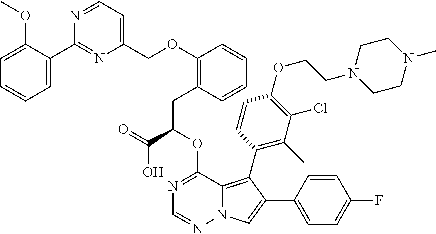

To a solution of methyl (2R)-2-{[(5Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoate (Step 4, P1, 33 mg, 0.04 mmol) in THE (4 mL) and water (1 mL) was added 2.0 M LiOH (1 mL). The reaction mixture was stirred at r.t. for 2 h. and adjusted with 1.0 N HCl to pH 5-6. The mixture was diluted with water (15 mL) and extracted with EA (3×20 mL). The organics were dried over sodium sulfate, filtered and concentrated under reduced pressure to give a crude product which was purified by Pre-HPLC on C18 column to afford (2R)-2-{[(5Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoic acid (15 mg, 46.2% yield). ee %=100% as showed by chiral HPLC with OD column. LCMS calculated for C₄₇H₄₆ClFN₇O₆ [M+H]⁺: m/z=858.31; found: 858.0. ¹H NMR: (400 MHz, DMSO-d₆) δ 8.84 (d, J=5.6 Hz, 1H), 8.04 (s, 1H), 7.90 (s, 1H), 7.81 (d, J=5.3 Hz, 1H), 7.59 (dd, J=7.6, 1.6 Hz, 1H), 7.50-7.45 (m, 1H), 7.22-7.13 (m, 4H), 7.05-7.02 (m, 1H), 7.00-6.93 (m, 4H), 6.88 (d, J=8.4 Hz, 1H), 6.69 (t, J=7.4 Hz, 1H), 6.18 (dd, J=7.5, 1.4 Hz, 1H), 5.57 (dd, J=10.2, 2.8 Hz, 1H), 5.27 (dd, J=2.4, 15.5 Hz, 2H), 4.31-4.26 (m, 2H), 3.85 (s, 3H), 3.57-3.47 (m, 2H), 3.36-3.32 (m, 2H), 3.30-3.26 (m, 3H), 3.31-3.12 (m, 1H), 3.06-3.03 (m, 3H), 2.82 (s, 3H), 2.70-2.64 (m, 1H), 2.40 (s, 3H).

### Example 10

### (2R)-2-{[(5Ra)-5-{3-Chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoic acid

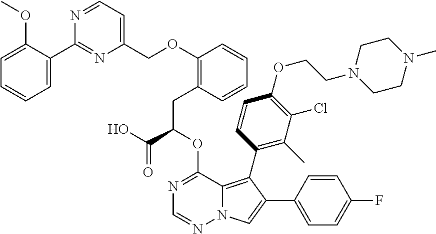

### Step 1: Methyl (2R)-2-{([(5Ra)-5-{β-chloro-2-methyl-4-[2-(4 methylpiperazin-1-yl)ethoxy]phenyl}-6-(4 fluorophenyl)pyrrolo[2,1-f][1,2,4]triazin-4-yl]oxy}-3-(2-{[2-(2-methoxyphenyl)pyrimidin-4-yl]methoxy}phenyl)propanoate

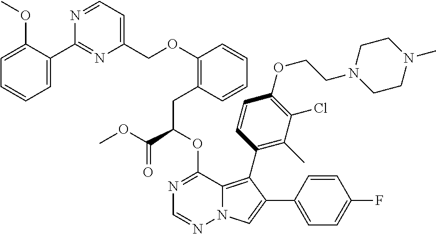

Methyl (2R)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoate (P2, 95 mg, Example 9 Step 4) was purified by Chiral HPLC to afford P2-1 (the earlier eluted product, 70 mg, Retention time t=2.998 min. in the chiral analytic AD-H column) and P2-2 (the latter eluted product, 6 mg, Retention time Rₜ=4.319 min. in the chiral analytic AD-H column) under Chiral HPLC separation conditions: Instrument: Waters-SFC80; Column: AD-H (30*250 mm, 5 um); Mobile phase A: Supercritical CO₂, Mobile phase B: EtOH (0.1% NH₃. H₂O), A:B=60/40; Flow rate: 50 mL/min; Circle Time: 15 min; Sample preparation: ethanol; Injection Volume: 1.0 mL; Detector Wavelength: 220 nm; Column temperature: 38° C.; Back pressure: 100 bar. The separated products were determined by chiral HPLC. Chiral HPLC conditions: Chiral Column: AD-H, 5 um, 4.6 mm×150 mm; Mobile phase: Hep:EtOH (0.1% DEA)=60:40; Flow rate: 0.5 mL/min and Run time: 10 min.; Detector Wavelength: 254 nm. The P1 was assigned to the title product. ¹H NMR: (400 MHz, CD₃OD) δ 8.87 (d, J=5.3 Hz, 1H), 8.04 (s, 1H), 7.88 (s, 1H), 7.73 (d, J=5.2 Hz, 1H), 7.59 (dd, J=7.6, 1.7 Hz, 1H), 7.49-7.44 (m, 2H), 7.24-7.20 (m, 2H), 7.16-7.12 (m, 3H), 7.06-6.93 (m, 3H), 6.72 (t, J=7.1 Hz, 1H), 6.14 (dd, J=7.4, 1.5 Hz, 1H), 5.60 (dd, J=10.4, 2.8 Hz, 1H), 5.23 (dd, J=21.2, 15.0 Hz, 2H), 4.31 (t, J=4.6 Hz, 2H), 3.82 (s, 3H), 3.78 (s, 3H), 3.46 (dd, J=13.8, 2.8 Hz, 1H), 2.93 (t, J=5.2 Hz, 2H), 2.76-2.57 (m, 8H), 2.32 (s, 1H), 1.79 (s, 3H).

### Step 2: (2R)-2-{([(5Ra)-5-{3-chloro-2-methyl-4-[2-(4 methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoic acid

This compound was prepared using procedures analogous to those described for Example 9 Step 5 using methyl (2R)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoate (P2-1 Step 1, 70.0 mg) to afford (2R) 2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]oxy}-3-(2-{[2-(2-methoxyphenyl)pyrimidin-4-yl]methoxy}phenyl)propanoic acid (55 mg). ee %=98.8% as showed by chiral HPLC with OD column. LCMS calculated for C₄₇H₄₆ClFN₇O₆ [M+H]⁺: m/z=858.31; found: 858.4. ¹H NMR: (400 MHz, CD₃OD) δ 8.85 (d, J=5.3 Hz, 1H), 8.03 (s, 1H), 7.91 (s, 1H), 7.84 (d, J=5.3 Hz, 1H), 7.65 (dd, J=7.6, 1.8 Hz, 1H), 7.52-7.48 (m, 2H), 7.24-7.12 (m, 5H), 7.08-7.04 (m, 1H), 7.00-6.94 (m, 3H), 6.72 (t, J=7.2 Hz, 1H), 6.10 (dd, J=7.4, 1.5 Hz, 1H), 5.60 (dd, J=11.0, 1.4 Hz, 1H), 5.27 (dd, J=25.8, 15.2 Hz, 2H), 4.34 (t, J=5.0 Hz, 2H), 3.86 (s, 3H), 3.57-3.48 (m, 1H), 3.36-3.32 (m, 3H), 3.29-3.18 (m, 4H), 3.10-3.05 (m, 1H), 2.83 (s, 3H), 2.61 (dd, J=13.8, 10.8 Hz, 1H), 1.79 (s, 3H).

### Example 11

### (2S)-2-{[(5Ra)-5-{3-Chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid and (2R)-2-{[(5Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid

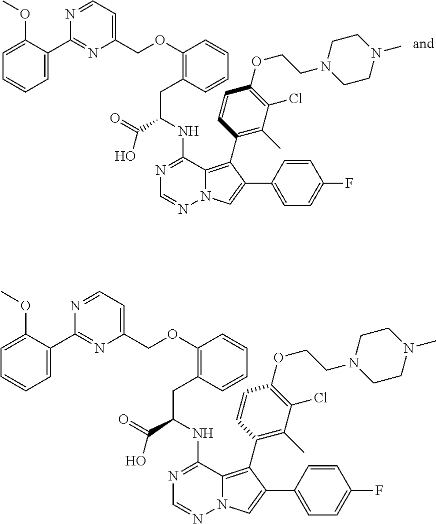

### Step 1: methyl (R)-2-[(tert-butoxycarbonyl)amino]-3-(2-{[2-(2-methoxyphenyl)pyrimidin-4-yl]methoxy}phenyl)propanoate and methyl (S)-2-[(tert-butoxycarbonyl)amino]-3-(2-{[2-(2-methoxyphenyl)pyrimidin-4-yl]methoxy}phenyl)propanoate

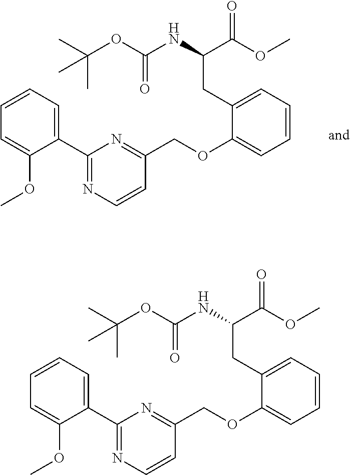 [SMILES: COC(=O)[C@@H](Cc1ccccc1OCc1ccnc(-c2ccccc2OC)n1)NC(=O)OC(C)(C)C ; COC(=O)[C@H](Cc1ccccc1OCc1ccnc(-c2ccccc2OC)n1)NC(=O)OC(C)(C)C]

A mixture of [2-(2-methoxyphenyl)pyrimidin-4-yl]methyl methanesulfonate (Example 9, Step 1, 360.0 mg, 1.22 mmol), a mixture of methyl (R)-2-[(tert-butoxycarbonyl)amino]-3-(2-hydroxyphenyl)propanoate and methyl (S)-2-[(tert-butoxycarbonyl)amino]-3-(2-hydroxyphenyl)propanoate (Intermediate 4, 361 mg, 1.2 mmol) and K₂CO₃ (350 mg, 2.5 mmol) in MeCN (20 mL) was stirred at 70° C. for 18 h. The solvent was removed under reduced pressure and the residue was purified by flash chromatography on a silica gel column (PE/EA=1/3) to give a mixture of methyl (R)-2-[(tert-butoxycarbonyl)amino]-3-(2-{[2-(2-methoxyphenyl)pyrimidin-4-yl]methoxy}phenyl)propanoate and methyl (S)-2-[(tert-butoxycarbonyl)amino]-3-(2-{[2-(2-methoxyphenyl)pyrimidin-4-yl]methoxy}phenyl)propanoate (350 mg, 58% yield) as a light oil. ¹H NMR: (400 MHz, DMSO-d₆) δ 8.94 (d, J=4.8 Hz, 1H), 7.63 (d, J=5.2 Hz, 1H), 7.56 (dd, J=7.6, 1.6 Hz, 1H), 7.47 (ddd, J=8.4, 7.6, 1.6 Hz, 1H), 7.32 (d, J=8.4 Hz, 1H), 7.23 (m, 2H), 7.16 (d, J=7.6 Hz, 1H), 7.08-7.04 (m, 2H), 6.92 (t, J=7.6 Hz, 1H), 5.31-5.23 (m, 2H), 4.38-4.33 (m, 1H), 3.77 (s, 3H), 3.63 (s, 3H), 3.30-3.24 (m, 1H), 2.85 (dd, J=13.2, 10.0 Hz, 1H), 1.31 (s, 9H).

### Step 2: methyl (R)-2-amino-3-(2-{[2-(2-methoxyphenyl)pyrimidin-4-yl]methoxy}phenyl)propanoate and methyl (S)-2-amino-3-(2-{([2-(2 methoxyphenyl)pyrimidin-4-yl]methoxy}phenyl)propanoate

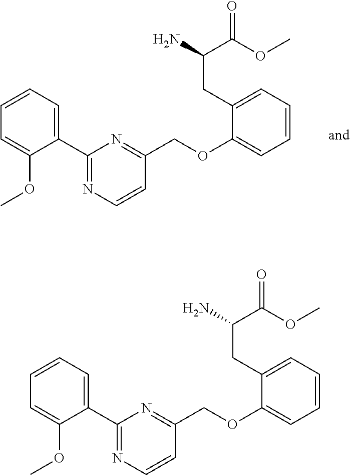 [SMILES: COC(=O)[C@H](N)Cc1ccccc1OCc1ccnc(-c2ccccc2OC)n1 ; COC(=O)[C@@H](N)Cc1ccccc1OCc1ccnc(-c2ccccc2OC)n1]

To a solution of methyl (R)-2-[(tert-butoxycarbonyl)amino]-3-(2-{[2-(2-methoxyphenyl)pyrimidin-4-yl]methoxy}phenyl)propanoate and methyl (S)-2-[(tert-butoxycarbonyl)amino]-3-(2-{[2-(2-methoxyphenyl)pyrimidin-4-yl]methoxy}phenyl)propanoate (350 mg, 0.71 mmol) in 1,4-dioxane (10 mL) was added HCl/Dioxane (4.0 N, 3.9 mL, 15 mmol) at r.t., the reaction mixture was stirred at r.t. for 3 h. The solvent was removed under reduced pressure to give the crude product as a pale white solid. The crude solid was washed with McOBu-t/DCM (40 mL/3 mL) to afford a mixture of methyl (R)-2-amino-3-(2-{[2-(2-methoxyphenyl)pyrimidin-4-yl]methoxy}phenyl)propanoate and methyl (S)-2-amino-3-(2-{[2-(2-methoxyphenyl)pyrimidin-4-yl]methoxy}phenyl)propanoate as HCl salt (230 mg, 69.5% yield) as a white solid. ¹H NMR: (400 MHz, DMSO-d6) δ 8.94 (d, J=4.8 Hz, 1H), 8.60 (br s, 2H), 7.64 (d, J=5.2, Hz, 1H), 7.56 (dd, J=7.6, 2.0 Hz, 1H), 7.48 (ddd, J=8.4, 7.6, 2.0 Hz, 1H), 7.30 (td, J=7.6, 1.6 Hz, 1H), 7.23 (dd, J=7.6, 1.2 Hz, 1H), 7.17 (d, J=8.0 Hz, 1H), 7.10 (d, J=8.8 Hz, 1H), 7.06 (dd, J=7.6, 0.8 Hz, 1H), 6.95 (td, J=8.0, 0.8 Hz, 1H), 5.28 (s, 2H), 5.07 (br s, 2H), 4.28-4.20 (m, 1H), 3.77 (s, 3H), 3.60 (s, 3H), 3.31-3.18 (m, 2H).

### Step 3: methyl (R)-2-{[5-bromo-6-(4 fluorophenyl)pyrrolo[2,1f][1,2,4]triazin-4-yl]amino}-3-[2-{[2-(2 methoxyphenyl)pyrimidin-4-yl]methoxy}phenyl]propanoate and methyl (S)-2-{[5-bromo-6-(4 fluorophenyl)pyrrolo[2,1f][1,2,4]triazin-4-yl]amino}-3-[2-{[2-(2-methoxyphenyl)pyrimidin-4-yl]methoxy}phenyl]propanoate

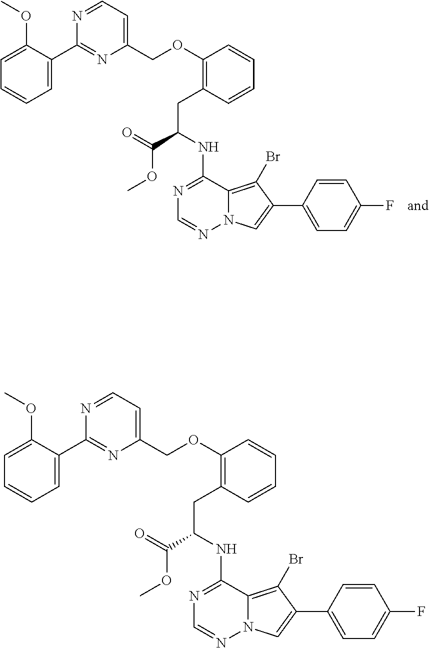 [SMILES: COC(=O)[C@@H](Cc1ccccc1OCc1ccnc(-c2ccccc2OC)n1)Nc1ncnn2cc(-c3ccc(F)cc3)c(Br)c12 ; COC(=O)[C@H](Cc1ccccc1OCc1ccnc(-c2ccccc2OC)n1)Nc1ncnn2cc(-c3ccc(F)cc3)c(Br)c12]

To a solution of methyl (R)-2-amino-3-(2-{[2-(2-methoxyphenyl)pyrimidin-4-yl]methoxy}phenyl)propanoate and methyl (S)-2-amino-3-(2-{[2-(2-methoxyphenyl)pyrimidin-4-yl]methoxy}phenyl)propanoate HCl salt (230 mg, 0.49 mmol), 5-bromo-4-chloro-6-(4-fluorophenyl)pyrrolo[2,1-f][1,2,4]triazine (Intermediate 1, 180 mg, 0.55 mmol) and TEA (0.5 mL, 3.6 mmol) in toluene (8 mL) was stirred at 110° C. for 18 h. The reaction mixture was concentrated under reduced pressure. The residue was purified by flash chromatography on a silica gel column (PE/EA=1/2) to give a mixture of methyl (R)-2-{[5-bromo-6-(4-fluorophenyl)pyrrolo[2,1-f][1,2,4]triazin-4-yl]amino}-3-[2-{[2-(2-methoxyphenyl)pyrimidin-4-yl]methoxy}phenyl]propanoate and methyl (S)-2-{[5-bromo-6-(4-fluorophenyl)pyrrolo[2,1-f][1,2,4]triazin-4-yl]amino}-3-[2-{[2-(2-methoxyphenyl)pyrimidin-4-yl]methoxy}phenyl]propanoate (270 mg, 70% yield) as a white solid. ¹H NMR: (400 MHz, DMSO-d₆) δ 8.83 (d, J=5.1 Hz, 1H), 7.93 (d, J=4.0 Hz, 2H), 7.57-7.53 (m, 2H), 7.47-7.41 (m, 3H), 7.28-7.22 (m, 5H), 7.12 (t, J=8.8 Hz, 2H), 7.00-6.93 (m, 2H), 5.27-5.14 (m, 3H), 3.74 (s, 3H), 3.71 (s, 3H), 3.61-3.58 (m, 1H), 3.29-3.27 (m, 2H).

### Step 4: methyl (2S)-2-{[(5Ra)-5-{3-chloro-2-methyl-4-[2-(4-methylpiperazin-1-yl)ethoxy]phenyl}-6-(4 fluorophenyl)pyrrolo[2,1f][1,2,4]triazin-4-yl]amino}-3-(2-{([2-(2 methoxyphenyl)pyrimidin-4-yl]methoxy}phenyl)propanoate and its enantiomer methyl (2R)-2-{[(5Sa)-5-{3-chloro-2-methyl-4-[2-(4-methylpiperazin-1-yl)ethoxy]phenyl}-6-(4 fluorophenyl)pyrrolo[2,1f][1,2,4]triazin-4-yl]amino}-3-(2-{([2-(2-methoxyphenyl)pyrimidin-4-yl]methoxy}phenyl)propanoate and methyl (2R)-2-{([(5Ra)-5-{3-chloro-2-methyl-4-[2-(4-methylpiperazin-1-yl)ethoxy]phenyl}-6-(4-fluorophenyl)pyrrolo[2,1f][1,2,4]triazin-4-yl]amino}-3-(2-{[2-(2-methoxyphenyl)pyrimidin-4-yl]methoxy}phenyl)propanoate and its enantiomer methyl (2S)-2-{[(5Sa)-5-{3-chloro-2-methyl-4-[2-(4-methylpiperazin-1-yl)ethoxy]phenyl}-6-(4 fluorophenyl)pyrrolo[2,1f][1,2,4]triazin-4-yl]amino}-3-(2-{[2-(2-methoxyphenyl)pyrimidin-4-yl]methoxy}phenyl)propanoate

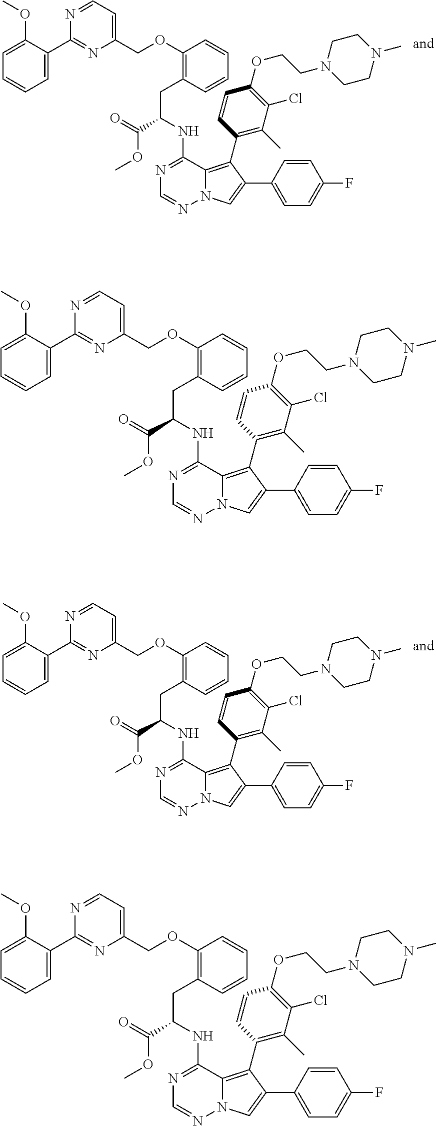

To a solution of methyl (R)-2-{[5-bromo-6-(4-fluorophenyl)pyrrolo[2,1-f][1,2,4]triazin-4-yl]amino}-3-[2-{[2-(2-methoxyphenyl)pyrimidin-4-yl]methoxy}phenyl]propanoate and methyl (S)-2-{[5-bromo-6-(4-fluorophenyl)pyrrolo[2,1-f][1,2,4]triazin-4-yl]amino}-3-[2-{[2-(2-methoxyphenyl)pyrimidin-4-yl]methoxy}phenyl]propanoate (270 mg, 0.40 mmol) and 1-[2-[2-chloro-3-methyl-4-(4,4,5,5-tetramethyl-1,3,2-dioxaborolan-2-yl)phenoxy]ethyl]-4-methyl-piperazine (Intermediate 2, 312 mg, 0.79 mmol) in THE (6 mL) and water (3 mL) was added chloro(2-dicyclohexylphosphino-2′,4′,6′-triisopropyl-1,1′-biphenyl)[2-(2-aminoethyl)phenyl)]palladium(II) (14.59 mg, 0.02 mmol)) and potassium phosphate (167 mg, 0.79 mmol) at r.t. in a seal tube, the reaction mixture was de-gassed and recharged with N₂ for three cycles and stirred at 70° C. for 2.5 h. under microwave irradiation. The mixture was concentrated under reduced pressure. The residue was purified by flash chromatography on a silica gel column (DCM:McOH=10:1) to afford two products which were further purified by Pre-HPLC on C18 column to afford P1 (the earlier eluted product, 60 mg) and P2 (the latter eluted product, 55 mg).

P1 contains two compounds in a ratio of 45:55 (Retention time: Rt=5.178 and 5.874, respectively) as showed by Chiral analytic HPLC in a AD column. Two compounds were assigned to methyl (2S)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoate and its enantiomer methyl (2R)-2-{[(5Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoate. ¹H NMR: (400 MHz, DMSO-d₆) δ 8.87 (d, J=5.2 Hz, 1H), 8.08 (s, 1H), 7.93 (s, 1H), 7.51 (dd, J=7.6, 1.8 Hz, 1H), 7.27-7.23 (m, 1H), 7.18-7.13 (m, 3H), 7.09-6.86 (m, 8H), 6.69 (dd, J=7.4, 1.2 Hz, 1H), 5.29-5.27 (m, 1H), 5.15-5.10 (m, 3H), 4.21-4.00 (m, 3H), 3.74 (s, 3H), 3.57 (s, 3H), 3.44-3.35 (m, 2H), 2.84-2.67 (m, 3H), 2.53-2.51 (m, 1H), 2.36-2.32 (m, 4H), 2.13 (s, 3H), 2.01 (s, 3H).

And P2 contains two compounds in a ratio of 55:45 (Retention time: Rt=5.566 and 6.460, respectively) as showed by Chiral analytic HPLC in a AD column. Two compounds were assigned to methyl (2R)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoate and its enantiomer methyl (2S)-2-{[(5 Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoate. ¹H NMR: (400 MHz, DMSO-d₆) δ 8.91 (d, J=5.2 Hz, 1H), 8.14 (s, 1H), 7.93 (s, 1H), 755-7.44 (m, 3H), 7.30-6.99 (m, 11H), 6.81-6.77 (m, 1H), 6.51-6.48 (m, 1H), 5.37 (d, J=7.6 Hz, 1H), 5.17 (s, 2H), 5.05-5.00 (m, 1H), 4.26-4.22 (m, 2H), 3.76 (s, 3H), 3.65 (s, 3H), 3.41-3.37 (m, 1H), 2.79-2.67 (m, 4H), 2.54-2.50 (m, 2H), 2.33-2.31 (m, 4H), 2.12 (s, 3H), 1.74 (s, 3H).

### Step 5: (2S)-2-{([(5Ra)-5-{3-chloro-2-methyl-4-[2-(4-methylpiperazin-1-yl)ethoxy]phenyl}-6-(4-fluorophenyl)pyrrolo[2,1f][1,2,4]triazin-4-yl]amino}-3-(2-{([2-(2-methoxyphenyl)pyrimidin-4-yl]methoxy}phenyl)propanoic acid and its enantiomer (2R)-2-{[(5Sa)-5-{3-chloro-2 methyl-4-[2-(4-methylpiperazin-1-yl)ethoxy]phenyl}-6-(4 fluorophenyl)pyrrolo[2,1f][1,2,4]triazin-4-yl]amino}-3-(2-{[2-(2-methoxyphenyl)pyrimidin-4-yl]methoxy}phenyl)propanoic acid

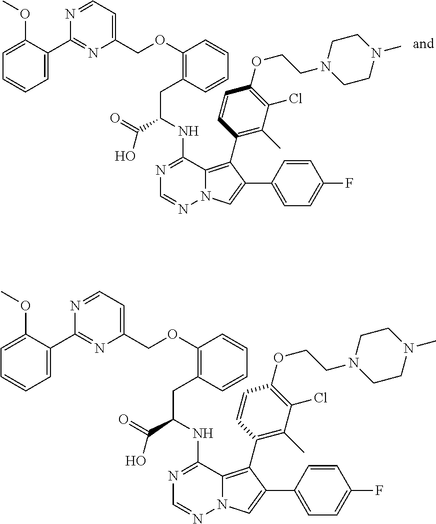

To a solution of methyl (2S)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoate and its enantiomer methyl (2R)-2 {[(5 Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoate (Step 4, P1, 60 mg, 0.07 mmol) in THE (6 mL) and water (1 mL) was added 2.0 M LiOH (1 mL). The reaction mixture was stirred at r.t. for 2 h. The mixture was adjusted with 1.0 M HCl to pH 5-6, diluted with water (15 mL), and extracted with EA (3×20 mL). The organics were dried over sodium sulfate, filtered and concentrated under reduced pressure. The residue was purified by Prep-HPLC on C18 column (5 um, 50 mm×150 mm, Mobile phase: H₂O (0.1% TFA)/CH₃CN) to afford (2S)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid and its enantiomer (2R)-2-{[(5 Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid (44 mg, 74.5% yield). LCMS: C₄₇H₄₇ClFN₈O₅ [M+H]⁺: m/z=857.3, found: 857.6. ¹H NMR: (400 MHz, DMSO-d₆) δ 8.82 (d, J=5.2 Hz, 1H), 8.05 (s, 1H), 7.93 (s, 1H), 7.52-7.36 (m, 3H), 7.26-7.13 (m, 4H), 7.08-7.01 (m, 4H), 6.97-6.85 (m, 3H), 6.76-6.74 (m, 1H), 5.32 (d, J=7.6 Hz, 1H), 5.11-5.02 (m, 4H), 4.27-4.09 (m, 4H), 3.74 (s, 3H), 3.50-3.41 (m, 3H), 3.11-2.95 (m, 4H), 2.81-2.75 (m, 4H), 2.67-2.60 (m, 1H), 2.03 (s, 3H).

### Example 12

### (2S)-2-{[(5Sa)-5-{3-Chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid

### And Example 13

### (2R)-2-{[(5Ra)-5-{3-Chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid

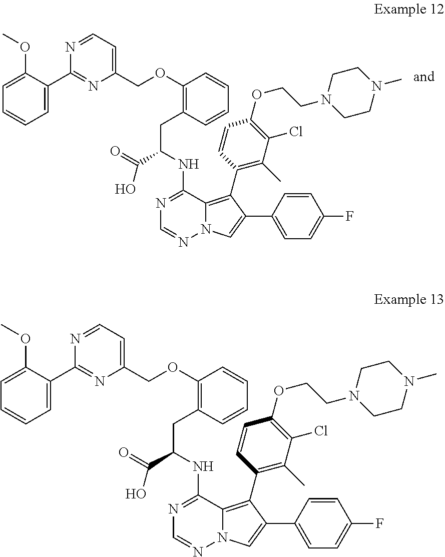

This compound was prepared using procedures analogous to those described for Example 11 Step 5 using methyl (2R)-2-[[5-[3-chloro-2-methyl-4-[2-(4-methylpiperazin-1-yl)ethoxy]phenyl]-6-(4-fluorophenyl)pyrrolo[2,1-f][1,2,4]triazin-4-yl]amino]-3-[2-[[2-(2-methoxyphenyl)pyrimidin-4-yl]methoxy]phenyl]propanoate (P2, 55 mg, Example 11 Step 4). The mixture of (2S)-2-{[(5 Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid and its enantiomer (2R)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid (31 mg, 55% yield) was purified by Chiral HPLC to afford P2-1 (the earlier eluted product, 3.4 mg, ee %=100%, Retention time Rt=5.189 min. in the chiral analytic OD-H column) and P2-2 (the latter eluted product, 5.0 mg, ee %=99.2%, Retention time t=7.899 min. in the chiral analytic OD-H coloumn) under Chiral HPLC separation conditions: Instrument: Waters-SFC80; Column: OD-H (30 mm×250 mm, 5 um); Mobile phase A: Supercritical CO₂, Mobile phase B: EtOH (0.1% NH₃H₂O), A:B=60/40; Flow rate: 50 mL/min; Circle Time: 15 min; Sample preparation: ethanol; Injection Volume: 1.0 mL; Detector Wavelength: 220 nm; Column temperature: 38° C.; Back pressure: 100 bar. The separated products were determined by chiral HPLC. Chiral HPLC conditions: Chiral Column: OD-H, 5 um, 4.6 mm×150 mm; Mobile phase: Hep:EtOH (0.1% DEA)=60:40; Flow rate: 0.5 mL/min and Run time: 10 min.; Detector Wavelength: 254 nm.

P2-1 was assigned to Example 12: (2S)-2-{[(5Sa)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid. LCMS: C₄₇H₄₇ClFN₈O₅ [M+H]⁺: m/z=857.3, found: 857.3. ¹H NMR: (400 MHz, DMSO-d6) δ 8.81 (d, J=5.2 Hz, 1H), 7.96 (s, 1H), 7.70 (s, 1H), 7.61-7.57 (m, 2H), 7.52-7.47 (m, 1H), 7.29-7.27 (m, 1H), 7.17-6.77 (m, 12H), 5.11-4.87 (m, 4H), 4.53-4.46 (m, 1H), 4.19-4.13 (m, 1H), 3.83 (s, 3H), 3.67-3.62 (m, 1H), 3.15-2.92 (m, 10H), 2.69 (s, 3H), 1.82 (s, 3H).

P2-2 was assigned to Example 13: (2R)-2-{[(5Ra)-5-{3-chloro-2-methyl-4-[2-(4-methylpiperazin-1-yl)ethoxy]phenyl}-6-(4-fluorophenyl)pyrrolo[2,1-f][1,2,4]triazin-4-yl]amino}-3-(2-{[2-(2-methoxyphenyl)pyrimidin-4-yl]methoxy}phenyl)propanoic acid. LCMS: C₄₇H₄₇ClFN₈O₅ [M+H]⁺: m/z=857.3, found: 857.3. ¹H NMR: (400 MHz, DMSO-d₆) δ 8.80 (d, J=5.2 Hz, 1H), 7.76 (s, 1H), 7.69 (s, 1H), 7.61-7.57 (m, 2H), 7.52-7.47 (m, 1H), 7.29-7.27 (m, 1H), 7.17-6.86 (m, 11H), 6.80-6.77 (m, 1H), 5.11-4.92 (m, 4H), 4.53-4.49 (m, 1H), 4.19-4.14 (m, 1H), 3.84 (s, 3H), 3.67-3.62 (m, 1H), 3.15-2.96 (m, 10H), 2.69 (s, 3H), 1.82 (s, 3H).

### Intermediate 1

### 5-Bromo-4-chloro-6-(4-fluorophenyl)pyrrolo[2,1-f][1,2,4]triazine

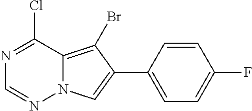 [SMILES: Fc1ccc(-c2cn3ncnc(Cl)c3c2Br)cc1]

### Step 1: methyl 4-(4 fluorophenyl)-1H pyrrole-2-carboxylate

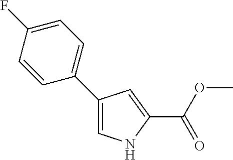 [SMILES: COC(=O)c1cc(-c2ccc(F)cc2)c[nH]1]

To a mixture of Pd(dppf)Cl₂.CH₂Cl₂ (5.38 g, 7.35 mmol), 4-fluorophenylboronic acid (223 g, 159 mmol) and methyl 4-bromo-1H-pyrrole-2-carboxylate (25 g, 122 mmol) in 1,4-dioxane (200 mL) and water (50 mL) was added potassium carbonate (33.8 g, 245 mmol). The mixture was degassed under vacuum and recharged with N₂ for three cycles. The reaction mixture was stirred at 100° C. for 18 h. The reaction mixture was cooled to room temperature, and water (50 mL) was added. The mixture was extracted with EA (3×100 mL). The combined organic layers were washed with brine, and then dried over sodium sulfate, filtered and concentrated under reduced pressure to give a crude product which was purified by chromatography on a silica gel column with (PE/EA=5:1) to give methyl 4-(4-fluorophenyl)-1H-pyrrole-2-carboxylate (16.9 g, 62.9% yield). ¹H NMR: (400 MHz, CDCl₃) δ 9.12 (br s, 1H), 7.48-7.44 (m, 2H), 7.17 (m, 1H), 7.14 (m, 1H), 7.07-7.03 (m, 2H), 3.89 (s, 3H).

### Step 2: methyl 3,5-dibromo-4-(4 fluorophenyl)-1H-pyrrole-2-carboxylate

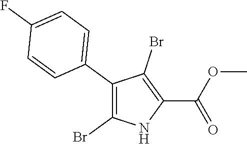 [SMILES: COC(=O)c1[nH]c(Br)c(-c2ccc(F)cc2)c1Br]

A suspension of N-bromosuccinimide (13 g, 73 mmol) in dry CHCl₃ (120 mL) was added to a solution of methyl 4-(4-fluorophenyl)-1H-pyrrole-2-carboxylate (8.0 g, 36.5 mmol) in CHCl₃ (10 mL) at room temperature. Subsequently, the reaction mixture was warmed to 85° C., and then stirred at this temperature for 18 h. After cooling, the reaction mixture was quenched with 10% Na₂S₂O₃/H₂O (80 mL), and then extracted with CHCl₃ (3×80 mL). The organic layers were dried over sodium sulfate, filtered and evaporated under reduced pressure. The residue was purified by chromatography on a silica gel column with PE:EA (from 10:0 to 10:3) to afford methyl 3,5-dibromo-4-(4-fluorophenyl)-1H-pyrrole-2-carboxylate (6.0 g, 43.6% yield) as a solid. ¹H NMR: (400 MHz, CDCl₃) δ 9.44 (br s, 1H), 7.41-7.37 (m, 2H), 7.16-7.11 (m, 2H), 3.93 (s, 3H).

### Step 3: methyl 3,5-dibromo-4-(4 fluorophenyl)-1-((2-(trimethylsilyl)ethoxy)methyl)-1H pyrrole-2-carboxylate

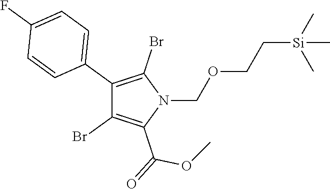 [SMILES: COC(=O)c1c(Br)c(-c2ccc(F)cc2)c(Br)n1COCC[Si](C)(C)C]

To a solution of methyl 3,5-dibromo-4-(4-fluorophenyl)-1H-pyrrole-2-carboxylate (10 g, 26 mmol) in dry THE (120 mL) at 0° C. under N₂ was added slowly 60% sodium hydride (1.59 g, 39.8 mmol) in small portions. The suspension was stirred at 0° C. for 30 min. To the suspension was added 2-(chloromethoxy)ethyl-trimethyl-silane (6.63 g, 39.8 mmol) at 0° C. under N₂. The reaction mixture was stirred at 0° C. for 2 h. and quenched with ice-water (20 mL) and extracted with EA (3×30 mL). The organics were dried over sodium sulfate, filtered and evaporated under reduced pressure. The residue was purified by chromatography on a silica gel column (PE:EA=10:0 to 10:1) to afford methyl 3,5-dibromo-4-(4-fluorophenyl)-1-(2-trimethylsilylethoxymethyl)pyrrole-2-carboxylate (9.5 g, 70.6% yield). ¹H NMR: (400 MHz, CDCl₃) δ 7.36-7.34 (m, 2H), 7.16-7.12 (m, 2H), 5.87 (s, 2H), 3.93 (s, 3H), 3.64-3.60 (m, 2H), 0.96-0.92 (m, 2H), 0.00 (s, 9H).

### Step 4: methyl 3-bromo-4-(4 fluorophenyl)-1-(2-trimethylsilylethoxymethyl)pyrrole-2-carboxylate

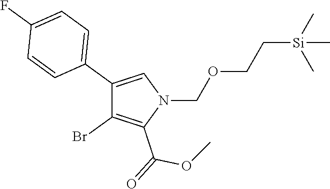 [SMILES: COC(=O)c1c(Br)c(-c2ccc(F)cc2)cn1COCC[Si](C)(C)C]

To a solution of methyl 3,5-dibromo-4-(4-fluorophenyl)-1-(2-trimethylsilylethoxymethyl)pyrrole-2-carboxylate (4.3 g, 8.4 mmol) in dry THE (8 mL) at 78° C. under N₂ was added n-butyllithium (4.1 mL, 10.2 mmol) (2.5 M in Hexane). The reaction mixture was stirred at −78° C. for 1 h. The mixture was carefully quenched with ice water (20 mL) and extracted with EA (3×20 mL). The organics were dried over sodium sulfate, filtered and evaporated. The residue was purified by chromatography on a silica gel column (PE:EA=10:0 to 9:1) to afford methyl 3-bromo-4-(4-fluorophenyl)-1-(2-trimethylsilylethoxymethyl)pyrrole-2-carboxylate (2.8 g, 77.1% yield). ¹H NMR: (400 MHz, CDCl₃) δ 7.49-7.46 (m, 2H), 7.13-7.08 (m, 3H), 5.68 (s, 2H), 3.93 (s, 3H), 3.59-3.55 (m, 2H), 0.96-0.92 (m, 2H), 0.00 (m, 9H).

### Step 5: 3-bromo-4-(4 fluorophenyl)-1H pyrrole-2-carboxylate

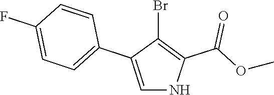 [SMILES: COC(=O)c1[nH]cc(-c2ccc(F)cc2)c1Br]

A solution of methyl 3-bromo-4-(4-fluorophenyl)-1-(2-trimethylsilylethoxymethyl)pyrrole-2-carboxylate (5.9 g, 13.8 mmol) in DCM (20 mL) was treated with trifluoroacetic acid (12.55 mL, 169 mmol). The mixture was stirred at r.t. overnight. The solvents were removed under reduced pressure. To the residue was added 40 mL of methanol and ammonia aqueous solution (28%, 10 mL). The mixture was stirred at r.t. for 2 h. The reaction mixture was concentrated under reduced pressure. The residue was extracted with EA (3×30 mL). The organics were dried over sodium sulfate, filtered and evaporated to give a crude product which was purified by chromatography on a silica gel column (PE:EA=10:1 to 2:1) to give the desired methyl 3-bromo-4-(4-fluorophenyl)-1H-pyrrole-2-carboxylate (2.6 g, 63.3% yield). ¹H NMR: (400 MHz, DMSO-d₆) δ 12.47 (s, 1H), 7.58-7.55 (m, 2H), 7.32-7.22 (m, 3H), 3.81 (s, 3H).

### Step 6: methyl 1-amino-3-bromo-4-(4 fluorophenyl)-1H pyrrole-2-carboxylate (HYS001-80)

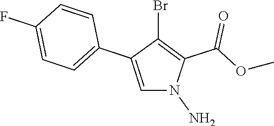 [SMILES: COC(=O)c1c(Br)c(-c2ccc(F)cc2)cn1N]

To a solution of methyl 3-bromo-4-(4-fluorophenyl)-1H-pyrrole-2-carboxylate (2.6 g, 8.7 mmol) in dry DMF (80 mL) was added NaH (1.22 g, 30.5 mmol) in small portions at 0° C. The suspension was stirred at 0° C. for 30 min. under nitrogen. To the suspension was added 0-(diphenylphosphinyl)hydroxylamine (8.1 g, 30 mmol) at 0° C. The reaction mixture was stirred at 0° C. under nitrogen for 3 h. and quenched with ice-water (60 mL). The mixture was extracted with EA (3×60 mL). The organics were dried over sodium sulfate, filtered and evaporated under reduced pressure. The residue was purified by chromatography on a silica gel column (PE:EA=10:0 to 2:1) to afford methyl 1-amino-3-bromo-4-(4-fluorophenyl)pyrrole-2-carboxylate (1.5 g, 36.8% yield).

### Step 7: 5-bromo-6-(4 fluorophenyl)pyrrolo[2,1-f][1,2,4]triazin-4(3H)-one

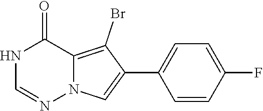 [SMILES: O=c1[nH]cnn2cc(-c3ccc(F)cc3)c(Br)c12]

A solution of methyl 1-amino-3-bromo-4-(4-fluorophenyl)pyrrole-2-carboxylate (1.4 g, 3.0 mmol) in formamide (5.4 mL, 134.5 mmol) was heated and stirred at 180° C. for 3 h. The mixture was cooled and added water (30 mL). The mixture was extracted with EA (3×40 mL). The organics were dried over sodium sulfate, filtered and evaporated under reduced pressure. The residue was purified by chromatography on a silica gel column (PE:EA=10:0 to 1:1) to afford 5-bromo-6-(4-fluorophenyl)-3H-pyrrolo[2,1-f][1,2,4]triazin-4-one (610 mg, 66.1% yield) as a grey solid. ¹HNMR (400 MHz, DMSO-d₆) δ 11.80 (s, 1H), 7.97 (s, 1H), 7.91 (s, 1H), 7.68-7.63 (m, 2H), 7.32-7.28 (m, 2H).

### Step 8: 5-bromo-4-chloro-6-(4 fluorophenyl)pyrrolo[2,1f][1,2,4]triazine

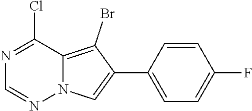 [SMILES: Fc1ccc(-c2cn3ncnc(Cl)c3c2Br)cc1]

A solution of 5-bromo-6-(4-fluorophenyl)-3H-pyrrolo[2,1-f][1,2,4]triazin-4-one (610 mg, 2 mmol) in POCl₃ (30 mL) was stirred at 100° C. for 18 h. The mixture was concentrated under reduced pressure to remove the excess POCl₃. The residue was purified by chromatography on a silica gel column (PE:EA=10:0 to 4:1) to give 5-bromo-4-chloro-6-(4-fluorophenyl)pyrrolo[2,1-f][1,2,4]triazine (360 mg, 55.7% yield) as a white solid. ¹H NMR: (400 MHz, DMSO-d₆) δ 8.65 (s, 1H), 8.49 (s, 1H), 7.75-7.72 (m, 2H), 7.40-7.35 (m, 2H).

### Intermediate 2

### 1-[2-[2-Chloro-3-methyl-4-(4,4,5,5-tetramethyl-1,3,2-dioxaborolan-2-yl)phenoxy]ethyl]-4-methyl-piperazine

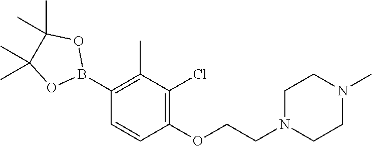

### Step 1: (4-bromo-2-chlorophenoxy)-trimethyl-silane

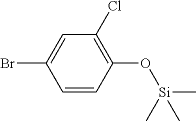

To a solution of 4-bromo-2-chloro-phenol (5.0 g, 24 mmol) in THF (30 mL) wad added hexamethyldisilazane (HMDS) (7.5 mL, 36 mmol) and the mixture was stirred at 75° C. for 2 h. The mixture was concentrated under reduced pressure to give of (4-bromo-2-chloro-phenoxy)-trimethyl-silane (6.74 g, quantitative) as a white solid which was used for the next step without further purification. ¹H NMR (400 MHz, CDCl₃) δ 7.47 (d, J=2.4 Hz, 1H), 7.34-7.13 (m, 1H), 6.73 (d, J=8.6 Hz, 1H), 0.26 (s, 9H).

### Step 2: 4-bromo-2-chloro-3-methyl-phenol

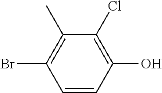

To a solution of (4-bromo-2-chloro-phenoxy)-trimethyl-silane (6.74 g, 24.1 mmol) in THF (50 mL) wad added lithium diisopropylamide (3.8 mL, 28.9 mmol) dropwise at −78° C. and the mixture was stirred at −78° C. for 2 h. Then Iodomethane (2.3 mL, 36 mmol) was added dropwise and the mixture was stirred at r.t. overnight. The mixture was quenched with sat. NH₄Cl (aq, 50 mL) and extracted with EA (2×30 mL). The combined organic layers were dried over Na₂SO₄, filtered, concentrated under reduced pressure to give the crude product which was washed with n-hexane to afford 4-bromo-2-chloro-3-methyl-phenol (2.0 g) as a white solid. ¹H NMR (400 MHz, CDCl₃) δ 7.36 (d, J=8.8 Hz, 1H), 6.79 (d, J=8.7 Hz, 1H), 5.58 (s, 1H), 2.50 (s, 3H).

### Step 3: (4-bromo-2-chloro-3-methyl phenoxy)-trimethyl silane

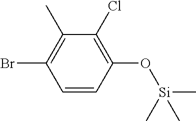

To a solution of 4-bromo-2-chloro-3-methyl-phenol (2.0 g, 9.0 mmol) in THF (20 mL) wad added HMDS (2.8 mL, 13 mmol). The mixture was stirred at 75° C. for 2 h. The mixture was concentrated under reduced pressure to give (4-bromo-2-chloro-3-methyl-phenoxy)-trimethyl-silane (2.6 g, 8.8 mmol) which was used for the next step without further purification. ¹H NMR (400 MHz, CDCl₃) δ 7.31 (d, J=8.7 Hz, 1H), 6.63 (d, J=8.7 Hz, 1H), 2.51 (s, 3H), 0.28 (s, 9H).

### Step 4: 2-chloro-3-methyl-4-(4,4,5,5-tetramethyl-1,3,2-dioxaborolan-2-yl)phenol

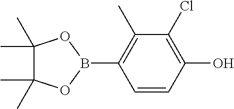 [SMILES: Cc1c(B2OC(C)(C)C(C)(C)O2)ccc(O)c1Cl]

To a solution of (4-bromo-2-chloro-3-methyl-phenoxy)-trimethyl-silane (2.6 g, 8.8 mmol) in THE (26 mL) wad added n-butyllithium (3.9 mL, 9.7 mmol) in hexane dropwise at −78° C. under Argon and the mixture was stirred at −78° C. for 5 min. Then 2-isopropoxy-4,4,5,5-tetramethyl-1,3,2-dioxaborolane (1.98 g, 10.6 mmol) was added dropwise and the mixture was stirred for 4 h. The mixture was quenched with NH₄Cl (aq, 50 mL) and extracted with EA (2×20 mL). The organic layer was dried over Na₂SO₄, filtered, concentrated under reduced pressure to give the crude product which was washed with n-hexane to afford 2-chloro-3-methyl-4-(4,4,5,5-tetramethyl-1,3,2-dioxaborolan-2-yl)phenol (1.2 g, 50.4% yield) as a white solid. ¹H NMR (400 MHz, DMSO-d₆) δ 10.35 (s, 1H), 7.42 (d, J=8.2 Hz, 1H), 6.80 (d, J=8.2 Hz, 1H), 2.49 (s, 3H), 1.27 (s, 12H).

### Step 5: 1-[2-[2-chloro-3-methyl-4-(4,4,5,5-tetramethyl-1,3,2-dioxaborolan-2-yl)phenoxy]ethyl]-4-methyl-piperazine

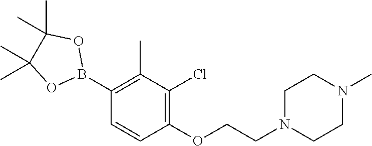

To a mixture of 2-chloro-3-methyl-4-(4,4,5,5-tetramethyl-1,3,2-dioxaborolan-2-yl)phenol (4.0 g, 15 mmol), triphenylphosphine (5.86 g, 22.3 mmol) and 1-(2-hydroxyethyl)-4-methylpiperazine (3.22 g, 22.3 mmol) in toluene (40 mL) was added diethyl azodicarboxylate (DEAD) (3.79 mL, 22.4 mmol). The reaction mixture was stirred at 50° C. overnight under Argon. The reaction mixture was concentrated under reduced pressure to give the crude product which was purified by chromatography on a silica gel column eluting with DCM:McOH=20:1 to give 1-[2-[2-chloro-3-methyl-4-(4,4,5,5-tetramethyl-1,3,2-dioxaborolan-2-yl)phenoxy]ethyl]-4-methyl-piperazine (4.76 g, 81% yield) as a whit solid. ¹H NMR (400 MHz, CDCl₃) δ 7.63 (d, J=8.3 Hz, 1H), 6.75 (d, J=8.3 Hz, 1H), 4.17 (t, J=5.9 Hz, 2H), 2.88 (t, J=5.9 Hz, 2H), 2.64 (d, J=27.6 Hz, 7H), 2.47 (s, 4H), 2.29 (s, 3H), 1.33 (s, 12H).

### Intermediate 3

### Methyl (R)-2-Hydroxy-3-(2-hydroxyphenyl)propanoate

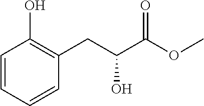

### Step 1: (R)-2-acetoxy-3-phenylpropanoic acid

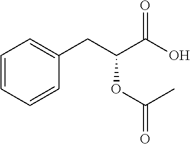

Acetic anhydride (17 mL, 180 mmol) was added to a solution of (2R)-2-hydroxy-3-phenyl-propanoic acid (25 g, 150 mmol) in pyridine (200 mL) at 25° C. The resulting mixture was stirred at 25° C. overnight. TLC (DCM:McOH=10:1, R_{f}=0.5) showed the reaction was completed. The solvent was removed under reduced pressure and the residue was adjusted with HCl (1 N) to pH 2˜3. The mixture was extracted with EA (3×200 mL). The combined organic layers were washed with brine (100 mL), dried by anhydrous Na₂SO₄, filtered and concentrated under reduced pressure to give (2R)-2-acetoxy-3-phenyl-propanoic acid (30.0 g, 95% yield) as a pale yellow oil. ¹H NMR (400 MHz, DMSO-d₆) δ 13.13 (s, 1H), 7.41-6.99 (m, 5H), 5.06 (dd, J=8.4, 4.4 Hz, 1H), 3.12 (dd, J=14.4, 4.4 Hz, 1H), 3.02 (dd, J=14.4, 8.4 Hz, 1H), 2.00 (s, 3H).

### Step 2: (R)-1-chloro-1-oxo-3-phenylpropan-2-yl acetate

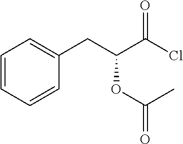

A solution of (2R)-2-acetoxy-3-phenyl-propanoic acid (30 g, 144 mmol) in thionyl chloride (55.1 mL, 759 mmol) was stirred at r.t. for 20 min. and then heated at 50° C. for 3 h. The reaction mixture was concentrated under reduced pressure to afford of [(1R)-1-benzyl-2-chloro-2-oxo-ethyl] acetate (29 g, 88.8% yield) as an amber-colored oil which was directly used for the next step without further purification.

### Step 3: (R)-1-oxo-2,3-dihydro-1H-inden-2-yl acetate

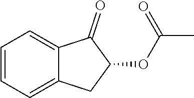

To a solution of AlCl₃ (44.1 g, 330 mmol) in DCM (200 mL) was added [(1R)-1-benzyl-2-chloro-2-oxo-ethyl] acetate (15 g, 66 mmol) at 0° C. The reaction was stirred at 25° C. for 3 h. TLC (PE:EA=5:1, R_{f}=0.3) showed the reaction was completed. The reaction mixture was diluted with ice-water (300 mL), filtered, extracted with DCM (2×300 mL). The organic layer was washed with brine (300 mL), dried over Na₂SO₄, filtered and concentrated under reduced pressure to give crude product which was purified by chromatography on a silica gel column (100-200 mesh size, PE:EA=20:1 to 5:1) to afford (R)-1-oxo-2,3-dihydro-1H-inden-2-yl acetate (7.1 g, 56.4% yield) as a white solid. ¹H NMR (400 MHz, DMSO-d₆) δ 7.74 (td, J=7.6, 0.8 Hz, 1H), 7.70 (d, J=7.6 Hz, 1H), 7.58 (d, J=7.6 Hz, 1H), 7.48 (t, J=7.6 Hz, 1H), 5.39 (dd, J=8.0, 4.4 Hz, 1H), 3.59 (dd, J=16.8, 8.0 Hz, 1H), 3.05 (dd, J=16.8, 4.4 Hz, 1H), 2.11 (s, 3H).

### Step 4: (R)-2-oxochroman-3-yl acetate

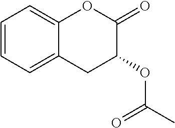

To a mixture of (R)-1-oxo-2,3-dihydro-1H-inden-2-yl acetate (7.1 g, 37 mmol) and CF₃SO₃H (0.56 g, 3.7 mmol) in DCM (60 mL) was added m-CPBA (6.4 g, 37 mmol) at 0° C. The mixture was stirred at 25° C. overnight. TLC (PE:EA=5:1, R_{f}=0.5) showed the reaction was completed. The reaction mixture was washed with NaHCO₃aq (3×200 mL) and brine (500 mL). The organic layer was dried over anhydrous Na₂SO₄, filtered, and concentrated under reduced pressure to give crude product which was purified by chromatography on a silica gel column (100-200 mesh size, PE:EA=50:1 to 7:1) to give [(3R)-2-oxochroman-3-yl] acetate (3.4 g, 44% yield) as a pale yellow solid. ¹H NMR (400 MHz, DMSO-d₆) δ 7.33 (m, 2H), 7.21-7.14 (m, 1H), 7.11 (dd, J=6.2, 3.1 Hz, 1H), 5.62 (dd, J=13.2, 6.8 Hz, 1H), 1H), 3.38 (dd, J=13.8, 13.2 Hz, 1H), 3.24 (dd, J=13.8, 6.8 Hz, 1H), 2.16 (s, 3H).

### Step 5: methyl (R)-2-hydroxy-3-(2-hydroxyphenyl)propanoate

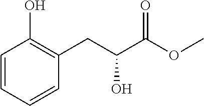

To a solution of [(3R)-2-oxochroman-3-yl] acetate (3.4 g, 16 mmol) in methanol (30 mL) was added K₂CO₃ (3.4 g, 24 mmol). The mixture was stirred at 25° C. overnight. TLC (PE:EA=3:1, R_{f}=0.5) showed the reaction was completed. The reaction mixture was adjusted with HCl (1 N) to pH=7, filtered and concentrated under reduced pressure to give crude product which was purified by chromatography on a silica gel column (100-200 mesh size, PE:EA=30:1 to 9:1) to give methyl (2R)-2-hydroxy-3-(2-hydroxyphenyl)propanoate (2.65 g, 81.91% yield) as a pale yellow oil. ¹H NMR (400 MHz, DMSO-d6) δ 9.50 (s, 1H), 7.15-6.91 (m, 2H), 6.76 (d, J=7.6 Hz, 1H), 6.72-6.64 (m, 1H), 5.52 (d, J=8.8 Hz, 1H), 4.27 (dd, J=8.1, 5.8 Hz, 1H), 3.54 (s, 3H), 2.93 (dd, J=13.4, 5.8 Hz, 1H), 2.71 (dd, J=13.4, 8.1 Hz, 1H).

### Step 6: (R)-2-(2 hydroxy-3-methoxy-3-oxopropyl)phenyl benzoate (QIZ002-28)

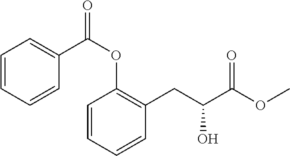 [SMILES: COC(=O)[C@H](O)Cc1ccccc1OC(=O)c1ccccc1]

To a solution of methyl (2R)-2-hydroxy-3-(2-hydroxyphenyl)propanoate (50 mg, 0.25 mmol) in DCM (3 mL) was added DIPEA (65.7 mg, 0.51 mmol), and then benzoyl chloride (35 mg, 0.25 mmol) was added dropwise at 0° C. The mixture was stirred at r.t. 16 h. TLC (PE:EA=3:1) showed the reaction worked well. The reaction was quenched by addition of water (5.0 mL). The mixture was extracted with DCM (2×10 mL). The organic layer was dried over Na₂SO₄, filtered and concentrated under reduced pressure to give crude product which was purified by chromatography on a silica gel column (PE:EA=7:1) to give [2-[(2R)-2-hydroxy-3-methoxy-3-oxo-propyl]phenyl] benzoate (26 mg, 33% yield). Analytic Chiral-HPLC showed no racemization happened during the reaction. The e.e. is about 91%: Peak 1 (4.37%, Retention time=7.205 min.) and Peak 2 (95.63%, Retention time=7.327 min.) as showed by Chiral analytic HPLC: Chiral Column: AD3, 5 um, 4.6 mm×250 mm; Mobile phase A: Supercritical CO₂, Mobile phase B: McOH (with 0.1% DEA); Flow rate: 1.0 mL/min.; Run time: 15 min.; Detector Wavelength: 254 nm.

### Intermediate 4

### Methyl (R)-2-((tert-butoxycarbonyl)amino)-3-(2-hydroxyphenyl)propanoate and Methyl (S)-2-((tert-butoxycarbonyl)amino)-3-(2-hydroxyphenyl)propanoate

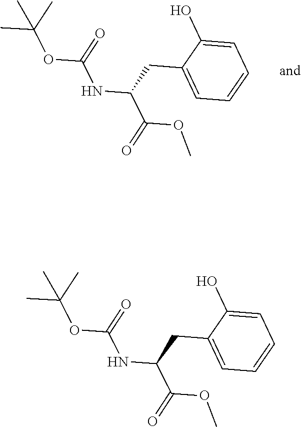 [SMILES: COC(=O)[C@@H](Cc1ccccc1O)NC(=O)OC(C)(C)C ; COC(=O)[C@H](Cc1ccccc1O)NC(=O)OC(C)(C)C]

### Step 1: methyl (R)-2-amino-3-(2 hydroxyphenyl)propanoate

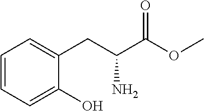

To a solution of (2R)-2-amino-3-(2-hydroxyphenyl)propanoic acid (5.0 g, 27 mmol) (Acrotein ChemBio, Cat. #: A-3168) in methanol (20 mL) was added HCl/MeOH (150.0 mL, 450 mmol) r.t. under N₂. The mixture was stirred at r.t. overnight, and concentrated under reduced pressure keeping the bath temperature below 40° C. to give methyl (2R)-2-amino-3-(2-hydroxyphenyl)propanoate as HCl salt (6.4 g, 100% yield) as a Red brown oil which was directly used in next step reaction without further purification. ¹H NMR: (400 MHz, DMSO-d6) δ 9.80 (br s. 1H), 8.54 (br s. 3H), 7.12-7.05 (m, 2H), 6.86 (d, J=8.0 Hz, 1H), 6.73 (t, J=7.6 Hz, 1H), 4.12 (br s, 1H), 3.74 (s, 3H), 3.05 (dd, J=2.0 Hz, 6.8 Hz, 2H).

### Step 2: methyl (R)-2-((tert-butoxycarbonyl)amino)-3-(2-hydroxyphenyl)propanoate and Methyl (S)-2-((tert-butoxycarbonyl)amino)-3-(2-hydroxyphenyl)propanoate

Methyl (2R)-2-amino-3-(2-hydroxyphenyl)propanoate HCl salt (6.4 g, 27.6 mmol) was suspended in DCM (100 mL). TEA (11.5 mL, 82 mmol) was added and the solution was cooled to 0° C. using a water-ice bath. A solution of (Boc)₂O (6.0 g, 27.6 mmol) in DCM (100 mL) was added slowly over 2.5 h. The mixture was stirred at r.t. overnight. Then 100 mL water was added. The organic phase was separated, washed with water, 1M HCl solution and finally with water again. The organic phase was dried over Na₂SO₄, filtered and concentrated under reduced pressure to afford methyl (2R)-2-(tert-butoxycarbonylamino)-3-(2-hydroxyphenyl)propanoate (major) and its enantiomer and Methyl (S)-2-((tert-butoxycarbonyl)amino)-3-(2-hydroxyphenyl)propanoate (minor) (5.5 g, 67% yield) as a white solid in a ratio of 60:40 (Retention time: t=3.10 and 3.73 min., respectively) as showed by Chiral analytic HPLC: Chiral Column: OD, 5 um, 3 mm×150 mm; Mobile phase A: Supercritical CO₂, Mobile phase B: i-PrOH, A:B=60:40; Run time: 10 min.; Detector Wavelength: 254 nm; Instrument: Waters Acouity UPC². ¹H NMR: (400 MHz, DMSO-d₆) δ 9.49 (s. 1H), 7.13 (d, J=8.0 Hz, 1H), 7.03 (t, J=7.6 Hz, 2H), 6.77 (d, J=7.6 Hz, 1 H), 6.68 (d, J=7.6 Hz, 1H), 4.27-4.21 (m, 1H), 3.57 (s, 3H), 3.00 (dd, J=5.6 Hz, 13.6 Hz, 1H), 2.71 (dd, J=9.6 Hz, 13.2 Hz, 1H), 1.31 (s, 9H).

### Biological Assays

### Cell Free Mcl-1: Bim Affinity Assay (Mcl-1 Bim)

The binding affinity of each compounds was measured via fluorescence polarization competition assay, in which the compound competes the same binding site with the ligand, and thus lead to dose-dependent anisotropy reduction. The tracer ligand utilized was a fluorescein isothiocyanate labelled peptide (FITC-ARIAQELRRIGDEFNETYTR) derived from Bim (GenScript).

The assay was carried out in black half-area 96-well NBS plate (Corning), containing 15 nM of MCL-1 (BPS Bioscience), 5 nM of FITC-Bim and 3-fold series diluted test compounds in a total volume of 50 uL of assay buffer (20 mM HEPES, 50 mM NaCl, 0.002% Tween 20 1 mM TCEP and 1% DMSO). The reaction plate was incubated for 1 hour at room temperature. The change of anisotropy is measured by Envision multimode plate reader (PerkinElmer) at emission wavelength 535 nm. Fluorescence polarization was calculated in mP unit and the percentage inhibition was calc. by % inhibition=100×(mP_{DMSO}−mP)/(mP_{DMSO}−mP_{PC}), in which mP_{DMSO} is the DMSO control, and mP_{PC} is the positive control. IC₅₀ values were determined from a 10-point dose response curve by fitting the percent inhibition against compound concentration using the GraphPad Prism software. The inhibition constant Kᵢ was subsequently calculated according Nikolovska-Coleska's equation (Anal. Biochem., 2004, 332, 261),

$${K}_{i} = \frac{{[ I ]}_{50}}{\frac{{[ L ]}_{50}}{{K}_{d}} + \frac{{[ P ]}_{0}}{{K}_{d}} + 1} \qquad$$

Where [I]₅₀ is the concentration of the free inhibitor at 50% inhibition, [L]₅₀ is the concentration of the free labeled ligand at 50% inhibition, [P]₀ is the concentration of the free protein at 0% inhibition, and K_{d} is the dissociation constant of the protein-ligand complex. See Table 2.

TABLE 2
Cell Free Mcl-1:Bim affinity assay (Mcl-1 Bim)

| Ex | BIM_Ki (nM) |
|---|---|
| 1 | 205 |
| 2 | >12000 |
| 3 | >12000 |
| 4 | 11.3 |
| 5 | >12000 |
| 6 | >12000 |
| 7 | 77.7 |
| 8 | >12000 |
| 9 | 8.2 |
| 10 | 0.22 |
| 11 | 382 |
| 12 | 990 |
| 13 | 0.7 |

### Caspase 3/7 Activity Assay

Dispense 10 uL aliquot of prepared H929 cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 2000 cells/well @ 5 uL/well. Transfer H929 cells to 50 mL conical (#430290, Corning) for each of the FBS concentration to be assayed (10%, 0.1%). Spin down at 1000 rpm for 5 min. using tabletop centrifuge (SPINCHRON 15, Beckman). Discard supernatant and resuspend cell pellet in modified RPMI 1640 (#10-040-CV, Corning) cell culture media containing sodium pyruvate (100 mM) (#25-000-CL, Corning), HEPES buffer (1M) (#25-060-CL, Corning) and glucose (200 g/1) (A24940-01, Gibco) with appropriate FBS (F2422-500 ML, Sigma) concentration to a cell density of 400,000 cells/mL. Dispense 5 uL of resuspended H929 cells per well in 384-well small volume TC treated plate (#784080, Greiner Bio-one) using standard cassette (#50950372, Thermo Scientific) on Multidrop Combi (#5840310, Thermo Scientific) in laminar flow cabinet. Dispense compounds onto plates using digital liquid dispenser (D300E, Tecan). Incubate plates in humidified tissue culture incubator @37° C. for 4 hours. Add 5 ul of prepared Caspase-Glo® 3/7 detection buffer (G8093, Promega) to each well of 384-well plate using small tube cassette (#24073295, Thermo Scientific) on Combi multi drop, incubate @ RT for 30-60 min. Read plates with microplate reader (PheraStar, BMG Labtech) using 384 well luminescence mode.

### Cell Viability Assay (H929 10 FBS)

Dispense 10 uL aliquot of prepared H929 cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 4000 cells/well @ 10 uL/well. Transfer H929 cells to 50 mL conical (#430290, Corning). Spin down at 1000 rpm for 5 min using tabletop centrifuge (SPINCHRON 15, Beckman). Discard supernatant and resuspend cell pellet in modified RPMI 1640 (#10-040-CV, Corning) cell culture media containing 10% FBS (F2422-500 ML, Sigma), sodium pyruvate (100 mM) (#25-000-CL, Corning), HEPES buffer (1M) (#25-060-CL, Corning) and glucose (200 g/L) (A24940-01, Gibco) to a cell density of 400,000 cells/mL. Dispense 10 uL of resuspended H929 cells per well in 384-well small volume TC treated plate (#784080, Greiner Bio-one) using standard cassette (#50950372, Thermo Scientific) on Multidrop Combi (#5840310, Thermo Scientific) in laminar flow cabinet. Dispense compounds onto plates using digital liquid dispenser (D300E, Tecan). Incubate plates in humidified tissue culture incubator @37° C. for 24 hours. Add 10 ul of prepared CellTiTer-Glo® detection buffer (G7570, Promega) or ATPlite 1 Step detection reagent (#6016731, Perkin Elmer) to each well of 384-well plate using small tube cassette (#24073295, Thermo Scientific) on Combi multi drop, incubate @ RT for 30-60 min. Read plates with microplate reader (PheraStar, BMG Labtech) using 384 well luminescence mode.

### Cytotoxicity Studies in NCI-H929 Cells

Cytotoxicity studies were conducted in NCI-H929 multiple myeloma cell line. Cells were maintained in RPMI 1640 (Corning Cellgro, Catalog #: 10-040-CV) supplemented with 10% v/v FBS (GE Healthcare, Catalog #: SH30910.03), 10 mM HEPES (Corning, Catalog #: 25-060-CI), 1 mM sodium pyruvate (Corning Cellgro, Catalog #: 25-000-CI and 2500 mg/L glucose (Gibco, Catalog #: A24940-01). Cells were seeded in 96-well plates at a density of 75000 cells/well. Compounds dissolved in DMSO were plated in duplicate using a digital dispenser (Tecan D300E) and tested on a 9-point 3-fold serial dilution. Cells were incubated for 24 hr in a 37° C. incubator at 5% CO₂. Cell viability was measured using the Cell Counting Kit-8 (CCK-8, Jojindo, CK04-13) as per manufacturer's instructions. Cells were incubated for 4 hr at 37° C. 5% CO₂ following addition of reagent and OD₄₅₀ values were measured with a microplate reader (iMark microplate reader, Bio-Rad). Background from media only wells were averaged and subtracted from all readings. OD₄₅₀ values were then normalized to DMSO controls to obtain percentage of viable cells, relative to DMSO vehicle control and plotted in Graphpad Prism ([Inhibitor] vs. normalized response−Variable slope; equation: Y=100/(1+(X\hat{ }HillSlope)/(IC₅₀\hat{ }HillSlope))) to determine IC₅₀ values (the concentration of compound inhibiting half of the maximal activity).

In some aspects, the disclosure is directed to the following aspects:

Aspect 1. A compound of Formula I:

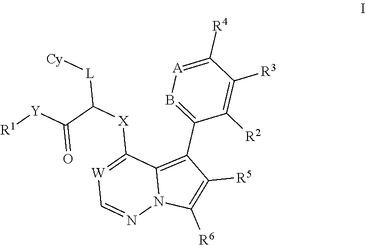

or a pharmaceutically acceptable salt or solvate thereof;

wherein

W is N or CR;

X is —CH₂—, —NH— or —O—;

Y is —NH— or —O—;

A is N, or CR⁷;

B is N, or CR⁸;

L is (CR⁹R¹⁰)ₘ, (CR⁹R¹⁰)ₚO(CR⁹R¹⁰)_{q}, (CR⁹R¹⁰)ₚS(CR⁹R¹⁰)_{q}, or (CR⁹R¹⁰)ₚNR¹¹(CR⁹R¹⁰)_{q};

Cy is aryl, heteroaryl, cycloalkyl, or heterocycloalkyl, each optionally substituted by 1, 2, 3, 4, or 5 R¹²;

R is H, halo, C₁-C₆ alkyl, C₁-C₆ haloalkoxy; C₂-C₆ alkenyl, C₂-C₆ alkynyl, C₁-C₆ haloalkyl, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};

R¹ is H, C₁-C₈ alkyl, aryl, heteroaryl, aryl-C₁-C₆ alkyl, or heteroaryl-C₁-C₆ alkyl, S(O)R^{B}, S(O)NR^{C}R^{D}, S(O)₂R^{B}, or S(O)₂NR^{C}R^{D};

each R², R³, R⁴, R⁷ and R⁸ is independently selected from H, halo, C₁-C₆ alkyl, C₂-C₆ alkenyl, C₂-C₆ alkynyl, C₁-C₆ haloalkyl, OH, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};

or R² and R³, or R³ and R⁴ together with the carbon atom to which they are attached form a 4- to 7-membered cycloalkyl group or 5- to 7-membered heterocycloalkyl group, or heteroaryl group, each optionally substituted by 1, 2, or 3 substituents independently selected from halo, C₁-C₆ alkyl, C₂-C₆ alkenyl, C₂-C₆ alkynyl, C₁-C₆ haloalkyl, —C₁-C₆ alkyl NR^{c}R^{d}, —C₁-C₆ alkyl-Cy¹, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, P(O)R^{e}R^{f}, P(O)OR^{e}OR^{f}, (S(O)R^{b}, S(O)NR^{c}R^{d}, S(O)₂R^{b}, NR^{c}S(O)₂R^{b}, or S(O)₂NR^{c}R^{d};

R⁵ is halo, C₁-C₆ alkyl, C₂-C₆ alkenyl, C₂-C₆ alkynyl, C₁-C₆ haloalkyl, CN, C(O)R^{B}, C(O)NR^{C}R^{D}, S(O)R^{B}, S(O)NR^{C}R^{D}, S(O)₂R^{B}, S(O)₂NR^{C}R^{D}, (CR⁹R¹⁰)ₘCy¹, C₂-C₆ alkenyl-Cy¹, C₂-C₆ alkynyl-Cy¹, C₂-C₆ alkynyl-O-Cy¹, (CR⁹R¹⁰)ₚO(CR⁹R¹⁰)_{q}Cy¹, (CR⁹R¹⁰)ₚS(CR⁹R¹⁰)_{q}Cy¹, (CR⁹R¹⁰)ₚNR¹¹(CR⁹R¹⁰)_{q}Cy¹, Cy²(CR⁹R¹⁰)ₘCy¹, Cy²(CR⁹R¹⁰)ₚO(CR⁹R¹⁰)_{q}Cy¹, Cy²(CR⁹R¹⁰)ₚS(CR⁹R¹⁰)_{q}Cy¹, or Cy²(CR⁹R¹⁰)ₚNR¹¹(CR⁹R¹⁰)₄Cy¹;

R⁶ is H, C₁-C₆ alkyl, C₂-C₆ alkenyl, C₂-C₆ alkynyl, C₁-C₆ haloalkyl, CN, NO₂, C(O)OR^{A}, C(O)R^{B}, C(O)NR^{C}R^{D}, S(O)R^{B}, S(O)NR^{C}R^{D}, S(O)₂R^{B}, or S(O)₂NR^{C}R^{D};

each R⁹ and R¹⁰ is independently selected from H, halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂;

or R⁹ and R¹⁰ together with the C atom to which they are attached form a 3, 4, 5, 6, or 7-membered cycloalkyl or heterocycloalkyl ring, each optionally substituted by 1, 2, or 3 substituents independently selected from halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂;

R¹¹ is H, C₁-C₆ alkyl, C₂-C₆ alkenyl, or C₂-C₆ alkynyl;

R¹² is H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{D}, P(O)R^{E}R^{F}, P(O)OR^{E}OR^{F}, S(O)R^{B}, S(O)NR^{C}R^{B}, S(O)₂R^{B}, NR^{C}S(O)₂R^{B}, S(O)₂NR^{C}R^{B}, Cy³, OCy³, O—C₁-C₆ alkyl-Cy³, or O—C₁-C₆ alkyl-Cy³-C₀-C₆ alkyl-Cy⁴;

wherein two adjacent R¹², together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{C}C(O)OR^{a}, OP(O)OR^{e}OR^{f}, P(O)OR^{e}OR^{f}, (S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl;

Cy¹ is aryl, heteroaryl, cycloalkyl, or heterocycloalkyl, each optionally substituted by 1, 2, 3, 4, or 5 R¹³;

each Cy², Cy³, and Cy⁴ are independently selected from aryl, cycloalkyl, heteroaryl, or heteorcycloalkyl;

R¹³ is H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{D}, P(O)R^{E}R^{F}, P(O)OR^{E}OR^{F}, S(O)R^{B}, S(O)NR^{C}R^{B}, S(O)₂R^{B}, NR^{C}S(O)₂R^{B}, S(O)₂NR^{C}R^{D}, aryl, cycloalkyl, heteroaryl, or heterocycloalkyl;

wherein two adjacent R¹³, together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, halosulfanyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{C}C(O)ORI, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl;

wherein the alkyl, alkenyl, alkynyl, haloalkyl, haloalkoxy and alkoxy groups of any of the R¹, R², R³, R⁴, R⁵, R⁶, R⁷, R⁸, R⁹, R¹⁰, and R¹¹ substituents can be unsubstituted or substituted with 1, 2, or 3 R¹⁴ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, B(OH)₂, OR^{a1}, SR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1}, NR^{c1}R^{d1}, NR_{c1}C(O)R^{b1}, NR^{c1}C(O)NR^{c1}R^{d1}, NR^{c1}C(O)OR^{a1}, P(O)OR^{e}OR^{f}, OP(O)OR^{e}OR^{f}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl;

wherein the aryl, heteroaryl, cycloalkyl and heterocycloalkyl groups, arylalkyl and heteroarylalkyl groups of any of the R¹, R⁹, R¹⁰, Cy², Cy³ and Cy⁴ groups can be unsubstituted or substituted with 1, 2, 3 or 4 R¹⁵ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, B(OH)₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR_{c1}C(O)R^{b1}, NR^{c1}C(O)NR^{c1}R^{d1}, NR^{c1}C(O)OR^{a1}, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl;

R^{A} is independently selected from H, C₁-C₆ alkyl, C₂-C₄ alkenyl, C₂-C₄ alkynyl, cycloalkyl, heterocycloalkyl, aryl, heteroaryl, or —C₁-C₆ alkyl-Cy³ wherein said C₁-C₄ alkyl, C₂-C₄ alkenyl, C₂-C₄ alkynyl, cycloalkyl, heterocycloalkyl, aryl or heteroaryl is optionally substituted with 1, 2, or 3 substituents independently selected from OH, CN, amino, halo, C₁-C₄ haloalkyl, or C₁-C₄ alkyl;

R^{B} is independently selected from H, C₁-C₆ alkyl, C₂-C₄ alkenyl, C₂-C₄ alkynyl, cycloalkyl, heterocycloalkyl, aryl, or heteroaryl, wherein said C₁-C₆ alkyl, C₂-C₄ alkenyl, C₂-C₄ alkynyl, cycloalkyl, heterocycloalkyl, aryl, or heteroaryl is optionally substituted with 1, 2, or 3 substituents independently selected from OH, CN, amino, halo, or C₁-C₄ alkyl;

each R^{C} and R^{D} are independently selected from H, C₁-C₆ alkyl, C₂-C₄ alkenyl, C₂-C₄ alkynyl, wherein said C₁-C₆ alkyl, C₂-C₄ alkenyl, C₂-C₄ alkynyl is optionally substituted with 1, 2, or 3 substituents independently selected from OH, CN, amino, halo, or C₁-C₄ alkyl;

or R^{C} and R^{D} together with the N atom to which they are attached form a 4-, 5-, 6- or 7-membered heterocycloalkyl group or heteroaryl group, each optionally substituted with 1, 2, or 3 substituents independently selected from OH, CN, amino, halo, or C₁-C₄ alkyl;

each R^{a} and R^{a1} are independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁-C₄ alkyl, C₁-C₄ alkoxy, C₁-C₄ haloalkyl, or C₁-C₄ haloalkoxy;

each R^{b} and R^{b1} are independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁-C₄ alkyl, C₁-C₄ alkoxy, C₁-C₄ haloalkyl, or C₁-C₄ haloalkoxy;

each R^{c} and R^{d} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl, wherein said C₁₋₄ alkyl, C₁₋₄ haloalkyl, C₂₋₄ alkenyl, C₂₋₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl is optionally substituted with 1, 2, or 3 substituents independently selected from OH, CN, amino, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, aryl, heteroaryl, C(O)OR^{a1}, C(O)R^{b1}, S(O)₂R^{b1}, alkoxyalkyl, and alkoxyalkoxy;

or R^{c} and R^{d} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁-C₄ alkyl, C₁-C₄ alkoxy, C₁-C₄ haloalkyl, C₁-C₄ haloalkoxy, hydroxyalkyl, cyanoalkyl, aryl, heteroaryl, C(O)OR^{a1}, C(O)R^{b1}, S(O)₂R^{b1}, alkoxyalkyl, and alkoxyalkoxy;

each R^{c1} and R^{d1} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl, wherein said C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁-C₄ alkyl, C₁-C₄ alkoxy, C₁-C₄ haloalkyl, or C₁-C₄ haloalkoxy;

or R^{c1} and R^{d1} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁-C₄ alkyl, C₁-C₄ alkoxy, C₁-C₄ haloalkyl, and C₁₋₄ haloalkoxy;

each R^{e} and R^{e1} are independently selected from H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl;

each R^{f} and R^{f1} are independently selected from H, C₁-C₄ alkyl, C₂-C₄ alkenyl, C₂-C₄ alkynyl, aryl, cycloalkyl, heteroaryl, or heterocycloalkyl;

each m is independently 0, 1, 2, 3, or 4;

each p is independently 0, 1, 2, 3, or 4; and

each q is independently 0, 1, 2, 3, or 4.

Aspect 2. The compound of aspect 1, having the Formula I-A:

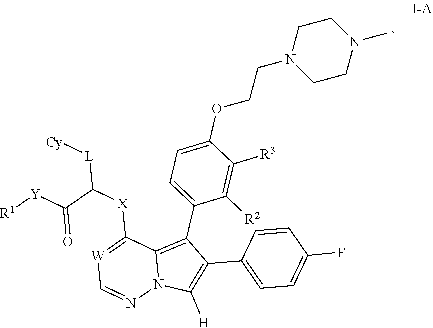

or a pharmaceutically acceptable salt or solvate thereof, wherein

W is N or CH;

X is NH or O;

Y is —NH— or —O—;

L is (—CH₂—)ₘ where m=0, 1, 2, 3, or 4;

Cy is aryl optionally substituted by 1, 2, 3, 4, or 5 R¹²;

R² is C₁-C₆ alkyl;

R³ is halo;

R¹² is OR^{A} wherein R^{A} is C₁-C₆ alkyl or C₁-C₆ alkyl substituted by 3 fluorine atoms; O—C₁-C₆ alkyl-Cy³ wherein Cy³ is cycloalkyl; O—C₁-C₆ alkyl-Cy³ wherein Cy³ is heteroaryl substituted by R¹⁵; or O—C₁-alkyl-Cy³-C₀ alkyl-Cy⁴ wherein Cy³ is pyrimidinyl and Cy⁴ is phenyl substituted by R¹⁵;

R¹⁵ is C₁-C₆ haloalkyl, or OR^{a1} wherein R^{a1} is C₁-C₄ alkyl; R¹ is H; C₁-C₈ alkyl substituted with OC(O)OR^{a1}, OC(O)NR^{c1}R^{d1}, or OP(O)OR^{e}OR^{f}; S(O)₂R^{B}, or S(O)₂NR^{C}R^{D};

R^{D} is C₁-C₆ alkyl or cycloalkyl; and

R_{C} and R^{D} are each independently C₁-C₆ alkyl; and

R^{a1}, R^{c1}, R^{d1}, R^{e}, and R^{f} are each C₁-C₄ alkyl.

Aspect 3. The compound of aspect 2, wherein W is N.

Aspect 4. The compound of aspect 2, wherein W is CH.

Aspect 5. The compound of any one of the preceding aspects, wherein X is O.

Aspect 6. The compound of any one of aspects 1-4, wherein X is NH.

Aspect 7. The compound of any one of the preceding aspects, wherein Y is O.

Aspect 8. The compound of any one of aspects 1-6, wherein Y is NH.

Aspect 9. The compound of any one of the preceding aspects, wherein L is —CH₂—.

Aspect 10. The compound of any one of the preceding aspects, wherein Cy is phenyl substituted by one R¹².

Aspect 11. The compound of aspect 10, wherein R¹² is —OCH₂CF₃; —O—CH₂-cyclopropyl,

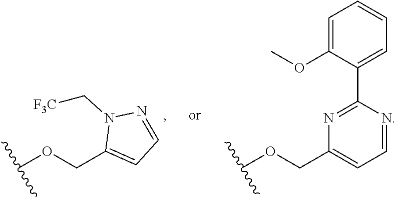 [SMILES: *OCc1ccnn1CC(F)(F)F ; *OCc1ccnc(-c2ccccc2OC)n1]

Aspect 12. The compound of any one of the preceding aspects, wherein R¹ is H.

Aspect 13. The compound of any one of aspects 1-11, wherein R¹ is S(O)₂R^{B}.

Aspect 14. The compound of aspect 13, wherein R^{B} is methyl, isopropyl, or cyclopropyl.

Aspect 15. The compound of any one of aspects 1-11, wherein R¹ is S(O)₂NR^{C}R^{D}.

Aspect 16. The compound of aspect 15, wherein R^{C} and R^{D} are both —CH₃.

Aspect 17. The compound of any one of aspects 1-11, wherein R¹ is C₁-C₈ alkyl substituted with —OC(O)OR^{a1}, —OC(O)NR^{c1}R^{d1}, or —OP(O)OR^{e}OR^{f}.

Aspect 18. The compound of aspect 17, wherein R¹ is

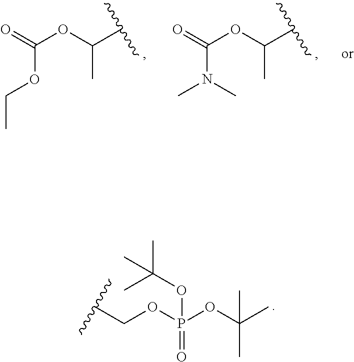

Aspect 19. The compound of any one of the preceeding aspects, wherein R² is —CH₃.

Aspect 20. The compound of any one of the preceeding aspects, wherein R³ is —Cl.

Aspect 21. A pharmaceutical composition comprising a compound according to any one of aspects 1 to 20 and a pharmaceutically acceptable excipient.

Aspect 22. A method of inhibiting a MCL-1 enzyme, comprising: contacting the MCL-1 enzyme with an effective amount of a compound of any one of any one of aspects 1 to 20.

Aspect 23. A method of treating a disease or disorder associated with aberrant MCL-1 activity in a subject comprising administering to the subject, a compound of any one of aspects 1 to 20.

Aspect 24. The method of aspect 23, wherein the disease or disorder associated with aberrant MCL-1 activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

1. A compound of Formula I: 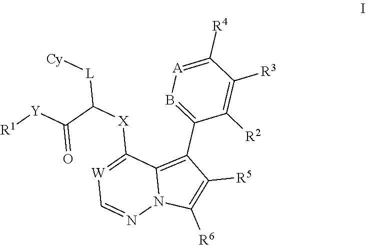
or a pharmaceutically acceptable salt or solvate thereof;
wherein
W is N or CR;
X is —CH₂—, —NH— or —O—;
Y is —NH— or —O—;
A is N, or CR⁷;
B is N, or CR⁸;
L is (CR⁹R¹⁰)ₘ, (CR⁹R¹⁰)ₚO(CR⁹R¹⁰)_{q}, (CR⁹R¹⁰)ₚS(CR⁹R¹⁰)_{q}, or (CR⁹R¹⁰)ₚNR¹¹(CR⁹R¹⁰)_{q};
Cy is aryl, heteroaryl, cycloalkyl, or heterocycloalkyl, each optionally substituted by 1, 2, 3, 4, or 5 R¹²;
R is H, halo, C₁-C₆ alkyl, C₁-C₆ haloalkoxy; C₂-C₆ alkenyl, C₂-C₆ alkynyl, C₁-C₆ haloalkyl, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};
R¹ is H, C₁-C₈ alkyl, aryl, heteroaryl, aryl-C₁-C₆ alkyl, or heteroaryl-C₁-C₆ alkyl, S(O)R^{B}, S(O)NR^{C}R^{D}, S(O)₂R^{B}, or S(O)₂NR^{C}R^{D};
each R², R³, R⁴, R⁷ and R⁸ is independently selected from H, halo, C₁-C₆ alkyl, C₂-C₆ alkenyl, C₂-C₆ alkynyl, C₁-C₆ haloalkyl, OH, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};
or R² and R³, or R³ and R⁴ together with the carbon atom to which they are attached form a 4- to 7-membered cycloalkyl group or 5- to 7-membered heterocycloalkyl group, or heteroaryl group, each optionally substituted by 1, 2, or 3 substituents independently selected from halo, C₁-C₆ alkyl, C₂-C₆ alkenyl, C₂-C₆ alkynyl, C₁-C₆ haloalkyl, —C₁-C₆ alkyl NR^{c}R^{d}, —C₁-C₆ alkyl-Cy¹, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, P(O)R^{e}R^{f}, P(O)OR^{e}OR^{f}, S(O)R^{b}, S(O)NR^{c}R^{d}, S(O)₂R^{b}, NR^{c}S(O)₂R^{b}, or S(O)₂NR^{c}R^{d};
R⁵ is halo, C₁-C₆ alkyl, C₂-C₆ alkenyl, C₂-C₆ alkynyl, C₁-C₆ haloalkyl, CN, C(O)R^{B}, C(O)NR^{C}R^{D}, S(O)R^{B}, S(O)NR^{C}R^{D}, S(O)₂R^{B}, S(O)₂NR^{C}R^{D}, (CR⁹R¹⁰)ₘCy¹, C₂-C₆ alkenyl-Cy¹, C₂-C₆ alkynyl-Cy¹, C₂-C₆ alkynyl-O-Cy¹, (CR⁹R¹⁰)ₚO(CR⁹R¹⁰)_{q}Cy¹, (CR⁹R¹⁰)ₚS(CR⁹R¹⁰)_{q}Cy¹, (CR⁹R¹⁰)ₚNR¹¹(CR⁹R¹⁰)_{q}Cy¹, Cy²(CR⁹R¹⁰)ₘCy¹, Cy²(CR⁹R¹⁰)ₚO(CR⁹R¹⁰)_{q}Cy¹, Cy²(CR⁹R¹⁰)ₚS(CR⁹R¹⁰)_{q}Cy¹, or Cy²(CR⁹R¹⁰)ₚNR¹¹(CR⁹R¹⁰)_{q}Cy¹;
R⁶ is H, C₁-C₆ alkyl, C₂-C₆ alkenyl, C₂-C₆ alkynyl, C₁-C₆ haloalkyl, CN, NO₂, C(O)OR^{A}, C(O)R^{B}, C(O)NR^{C}R^{D}, S(O)R^{B}, S(O)NR^{C}R^{D}, S(O)₂R^{B}, or S(O)₂NR^{C}R^{D};
each R⁹ and R¹⁰ is independently selected from H, halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂;
or R⁹ and R¹⁰ together with the C atom to which they are attached form a 3, 4, 5, 6, or 7-membered cycloalkyl or heterocycloalkyl ring, each optionally substituted by 1, 2, or 3 substituents independently selected from halo, OH, C₁-C₆ alkyl, C₂-C₆ alkenyl, C₂-C₆ alkynyl, C₁-C₆ alkoxy, C₁-C₆ alkoxy-C₁-C₆ alkyl, C₁-C₆ cyanoalkyl, heterocycloalkyl, cycloalkyl, C₁-C₆ haloalkyl, CN, or NO₂;
R¹¹ is H, C₁-C₆ alkyl, C₂-C₆ alkenyl, or C₂-C₆ alkynyl;
R¹² is H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{c}R^{D}, NR^{C}C(O)OR^{A}, P(O)R^{E}R^{F}, P(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, Cy³, OCy³, O—C₁-C₆ alkyl-Cy³, or O—C₁-C₆ alkyl-Cy³-C₀-C₆ alkyl-Cy⁴;
wherein two adjacent R¹², together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl;
Cy¹ is aryl, heteroaryl, cycloalkyl, or heterocycloalkyl, each optionally substituted by 1, 2, 3, 4, or 5 R¹³;
each Cy², Cy³, and Cy⁴ are independently selected from aryl, cycloalkyl, heteroaryl, or heteorcycloalkyl;
R¹³ is H, halo, C₁-C₆ alkyl, C₂-C₆ alkenyl, C₂-C₆ alkynyl, C₁-C₆ haloalkyl, CN, NO₂, N₃, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{D}, P(O)R^{E}R^{F}, P(O)OR^{E}OR^{F}, S(O)R^{B}, S(O)NR^{C}R^{D}, S(O)₂R^{B}, NR^{C}S(O)₂R^{B}, S(O)₂NR^{C}R^{D}, aryl, cycloalkyl, heteroaryl, or heterocycloalkyl;
wherein two adjacent R¹³, together with the atoms to which they are attached, optionally form a fused 4-10 membered cycloalkyl ring or a fused 4-10 membered heterocycloalkyl ring, each optionally substituted by 1, 2, or 3 substituents independently selected from halo, C₁-C₆ alkyl, C₂-C₆ alkenyl, C₂-C₆ alkynyl, C₁-C₆ haloalkyl, halosulfanyl, CN, NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{C}C(O)OR^{a}, OP(O)OR^{e}OR^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, aryl, cycloalkyl, heteroaryl, and heterocycloalkyl;
wherein the alkyl, alkenyl, alkynyl, haloalkyl, haloalkoxy and alkoxy groups of any of the R¹, R², R³, R⁴, R⁵, R⁶, R⁷, R⁸, R⁹, R¹⁰, and R¹¹ substituents can be unsubstituted or substituted with 1, 2, or 3 R¹⁴ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, B(OH)₂, OR^{a1}, SR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, P(O)OR^{e}R^{f}, OP(O)OR^{e}OR^{f}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl;
wherein the aryl, heteroaryl, cycloalkyl and heterocycloalkyl groups, arylalkyl and heteroarylalkyl groups of any of the R¹, R⁹, R¹⁰, Cy², Cy³ and Cy⁴ groups can be unsubstituted or substituted with 1, 2, 3 or 4 R¹⁵ substituents independently selected from halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, B(OH)₂, OR^{a1}, SR^{a1}, C(O)R^{b1}, C(O)NR^{c1}R^{d1}, C(O)OR^{a1}, OC(O)R^{b1}, OC(O)NR^{c1}R_{d1}, NR^{c1}R^{d1}, NR^{c1}C(O)R^{b1}, NR^{c1}C(O)NR^{c1}R^{d1}, NR^{c1}C(O)OR^{a1}, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1}, aryl, heteroaryl, cycloalkyl, or heterocycloalkyl;
R^{A} is independently selected from H, C₁-C₆ alkyl, C₂-C₄ alkenyl, C₂-C₄ alkynyl, cycloalkyl, cycloalkylalkyl, —C₁-C₆ alkyl-Cy³, heterocycloalkyl, heterocycloalkylalkyl, aryl, or heteroaryl, wherein said C₁-C₆ alkyl, C₂-C₄ alkenyl, C₂-C₄ alkynyl, cycloalkyl, cycloalkylalkyl, heterocycloalkyl, heterocycloalkylalkyl, aryl or heteroaryl is optionally substituted with 1, 2, or 3 substituents independently selected from OH, CN, halo, C₁-C₄ alkyl; NO₂, Oxo, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, OP(O)OR^{e}R^{f}, P(O)OR^{e}OR^{f}, S(O)R^{b}, S(O)NR^{c}R^{d}, S(O)₂R^{b}, NR^{c}S(O)₂R^{b}, S(O)₂NR^{c}R^{d};
R^{B} is independently selected from H, C₁-C₆ alkyl, C₂-C₄ alkenyl, C₂-C₄ alkynyl, cycloalkyl, heterocycloalkyl, aryl, or heteroaryl, wherein said C₁-C₆ alkyl, C₂-C₄ alkenyl, C₂-C₄ alkynyl, cycloalkyl, heterocycloalkyl, aryl, or heteroaryl is optionally substituted with 1, 2, or 3 substituents independently selected from OH, CN, amino, alkylamino, dialkylamino, halo, or C₁-C₄ alkyl;
each R^{C} and R^{D} are independently selected from H, C₁-C₆ alkyl, C₂-C₄ alkenyl, C₂-C₄ alkynyl, wherein said C₁-C₆ alkyl, C₂-C₄ alkenyl, C₂-C₄ alkynyl is optionally substituted with 1, 2, or 3 substituents independently selected from OH, CN, amino, alkylamino, dialkylamino, halo, or C₁-C₄ alkyl;
or R^{C} and R^{D} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, or C₁-C₄ alkyl;
R^{E} is independently H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl;
R^{F} is independently H, C₁-C₄ alkyl, C₂-C₄ alkenyl, C₂-C₄ alkynyl, aryl, cycloalkyl, heteroaryl, or heterocycloalkyl;
each R^{a} and R^{a1} are independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy;
each R^{b} and R^{b1} are independently selected from H, C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl, wherein said C₁-C₄ alkyl, C₁-C₄ haloalkyl, C₂-C₄ alkenyl, C₂-C₄ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy;
each R^{c} and R^{d} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl, wherein said C₁₋₄ alkyl, C₁₋₄ haloalkyl, C₂₋₄ alkenyl, C₂₋₄ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, or biheteroaryl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, aryl, heteroaryl, C(O)OR^{a1}, C(O)R^{b1}, S(O)₂R^{b1}, alkoxyalkyl, and alkoxyalkoxy;
or R^{c} and R^{d} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, C₁-C₄ haloalkoxy, hydroxyalkyl, cyanoalkyl, aryl, heteroaryl, C(O)OR^{a1}, C(O)R^{b1}, S(O)₂R^{b1}, alkoxyalkyl, and alkoxyalkoxy;
each R^{c1} and R^{d1} are independently selected from H, C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl, wherein said C₁-C₄ alkyl, C₁-C₄ haloalkyl, C₂-C₄ alkenyl, C₂-C₄ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, or C₁-C₄ haloalkoxy;
or R^{c1} and R^{d1} together with the N atom to which they are attached form a 4-, 5-, 6- or 7-membered heterocycloalkyl group or heteroaryl group, each optionally substituted with 1, 2, or 3 substituents independently selected from OH, CN, amino, alkylamino, dialkylamino, halo, C₁-C₄ alkyl, C₁-C₄ alkoxy, C₁-C₄ haloalkyl, and C₁₋₄ haloalkoxy;
each R^{e} and R^{e1} are independently selected from H, C₁-C₄ alkyl, C₁-C₄ haloalkyl, C₂-C₄ alkenyl, (C₁-C₄ alkoxy)-C₁-C₄ alkyl, C₂-C₄ alkynyl, aryl, cycloalkyl, heteroaryl, heterocycloalkyl, arylalkyl, cycloalkylalkyl, heteroarylalkyl, or heterocycloalkylalkyl;
each R^{f} and R^{f1} are independently selected from H, C₁-C₄ alkyl, C₂-C₄ alkenyl, C₂-C₄ alkynyl, aryl, cycloalkyl, heteroaryl, or heterocycloalkyl;
each m is independently 0, 1, 2, 3, or 4;
each p is independently 0, 1, 2, 3, or 4; and
each q is independently 0, 1, 2, 3, or 4.

2. The compound of claim 1, having the Formula I-A: 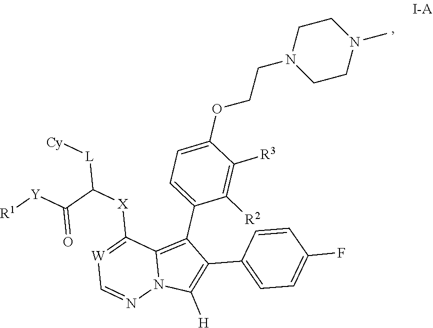
or a pharmaceutically acceptable salt or solvate thereof, wherein
W is N or CH;
X is NH or O;
Y is —NH— or —O—;
L is (—CH₂—)ₘ where m=0, 1, 2, 3, or 4;
Cy is aryl optionally substituted by 1, 2, 3, 4, or 5 R¹²;
R² is C₁-C₆ alkyl;
R³ is halo;
R¹² is OR^{A} wherein R^{A} is C₁-C₆ alkyl or C₁-C₆ alkyl substituted by 3 fluorine atoms; O—C₁-C₆ alkyl-Cy³ wherein Cy³ is cycloalkyl; O—C₁-C₆ alkyl-Cy³ wherein Cy³ is heteroaryl substituted by R¹⁵; or O—C₁-alkyl-Cy³-C₀ alkyl-Cy⁴ wherein Cy³ is pyrimidinyl and Cy⁴ is phenyl substituted by R¹⁵;
R¹⁵ is C₁-C₆ haloalkyl, or OR^{a1} wherein R^{a1} is C₁-C₄ alkyl;
R¹ is H, C₁-C₈ alkyl substituted with OC(O)OR^{a1}, OC(O)NR^{c1}R^{d1}, or OP(O)OR^{e}OR^{f}; S(O)₂R^{B}, or S(O)₂NR^{C}R^{D};
R^{B} is C₁-C₆ alkyl or cycloalkyl; and
R^{C} and R^{D} are each independently C₁-C₆ alkyl; and
R^{a1}, R^{c1}, R^{d1}, R^{e}, and R^{f} are each C₁-C₄ alkyl.

3. The compound of claim 2, wherein W is N.

4. The compound of claim 2, wherein W is CH.

5. The compound of claim 1, wherein X is O.

6. The compound of claim 1, wherein X is NH.

7. The compound of claim 1, wherein Y is O.

8. The compound of claim 1, wherein Y is NH.

9. The compound of claim 1, wherein L is —CH₂—.

10. The compound of claim 1, wherein Cy is phenyl substituted by one R¹².

11. The compound of claim 10, wherein R¹² is —OCH₂CF₃; —O—CH₂-cyclopropyl,

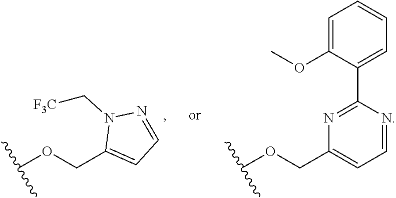 [SMILES: *OCc1ccnn1CC(F)(F)F ; *OCc1ccnc(-c2ccccc2OC)n1]

12. The compound of claim 1, wherein R¹ is H.

13. The compound of claim 1, wherein R¹ is S(O)₂R^{B}.

14. The compound of claim 13, wherein R^{B} is methyl, isopropyl, or cyclopropyl.

15. The compound of claim 1, wherein R¹ is S(O)₂NR^{C}R^{D}.

16. The compound of claim 15, wherein R^{C} and R^{D} are both —CH₃.

17. The compound of claim 1, wherein R¹ is C₁-C₈ alkyl substituted with —OC(O)OR^{a1}, —OC(O)NR^{c1}R^{d1}, or —OP(O)OR^{e}OR^{f}.

18. The compound of claim 17, wherein R¹ is

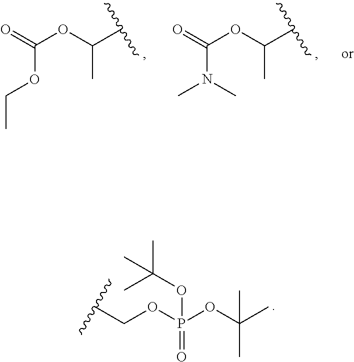

19. The compound of claim 1, wherein R² is —CH₃.

20. The compound of claim 1, wherein R³ is —Cl.

21. A pharmaceutical composition comprising a compound according to claim 1 and a pharmaceutically acceptable excipient.

22. A method of inhibiting a MCL-1 enzyme, comprising: contacting the MCL-1 enzyme with an effective amount of a compound of claim 1.

23. A method of treating a disease or disorder associated with aberrant MCL-1 activity in a subject comprising administering to the subject, a compound of claim 1.

24. The method of claim 23, wherein the disease or disorder associated with aberrant MCL-1 activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
